SEQUENCE LISTING (23 sequences)

SEQ ID NO: 1 - DNA, 2156 bp, Homo sapiens
  CDS (71)...(1900)
gcgcatgcgtggattgtcgtcttctgtccaagttggtcgcttccctgcgccaaagtgagcagtagccaacatgtcagggtgggagtcatattacaaaaccgagggcgatgaagaagcagaggaagaacaagaagagaaccttgaagcaagtggagactataaatattcaggaagagatagtttgatttttttggttgatgcctccaaggctatgtttgaatctcagagtgaagatgagttgacaccttttgacatgagcatccagtgtatccaaagtgtgtacatcagtaagatcataagcagtgatcgagatctcttggctgtggtgttctatggtaccgagaaagacaaaaattcagtgaattttaaaaatatttacgtcttacaggagctggataatccaggtgcaaaacgaattctagagcttgaccagtttaaggggcagcagggacaaaaacgtttccaagacatgatgggccacggatctgactactcactcagtgaagtgctgtgggtctgtgccaacctctttagtgatgtccaattcaagatgagtcataagaggatcatgctgttcaccaatgaagacaacccccatggcaatgacagtgccaaagccagccgggccaggaccaaagccggtgatctccgagatacaggcatcttccttgacttgatgcacctgaagaaacctgggggctttgacatatccttgttctacagagatatcatcagcatagcagaggatgaggacctcagggttcactttgaggaatccagcaagctagaagacctgttgcggaaggttcgcgccaaggagaccaggaagcgagcactcagcaggttaaagctgaagctcaacaaagatatagtgatctctgtgggcatttataatctggtccagaaggctctcaagcctcctccaataaagctctatcgggaaacaaatgaaccagtgaaaaccaagacccggacctttaatacaagtacaggcggtttgcttctgcctagcgataccaagaggtctcagatctatgggagtcgtcagattatactggagaaagaggaaacagaagagctaaaacggtttgatgatccaggtttgatgctcatgggtttcaagccgttggtactgctgaagaaacaccattacctgaggccctccctgttcgtgtacccagaggagtcgctggtgattgggagctcaaccctgttcagtgctctgctcatcaagtgtctggagaaggaggttgcagcattgtgcagatacacaccccgcaggaacatccctccttattttgtggctttggtgccacaggaagaagagttggatgaccagaaaattcaggtgactcctccaggcttccagctggtctttttaccctttgctgatgataaaaggaagatgccctttactgaaaaaatcatggcaactccagagcaggtgggcaagatgaaggctatcgttgagaagcttcgcttcacatacagaagtgacagctttgagaaccccgtgctgcagcagcacttcaggaacctggaggccttggccttggatttgatggagccggaacaagcagtggacctgacattgcccaaggttgaagcaatgaataaaagactgggctccttggtggatgagtttaaggagcttgtttacccaccagattacaatcctgaagggaaagttaccaagagaaaacacgataatgaaggttctggaagcaaaaggcccaaggtggagtattcagaagaggagctgaagacccacatcagcaagggtacgctgggcaagttcactgtgcccatgctgaaagaggcctgccgggcttacgggctgaagagtgggctgaagaagcaggagctgctggaagccctcaccaagcacttccaggactgaccagaggccgcgcgtccagctgcccttccgcagtgtggccaggctgcctggccttgtcctcagccagttaaaatgtgtttctcctgagctaggaagagtctacccgacataagtcgagggactttatgtttttgaggctttctgttgccatggtgatggtgtagccctcccactttgctgttccttactttactgcctgaataaagagccctaagtttgtactatatactgttaaaaaaaaaaaaaaaaaaaaaaa

SEQ ID NO: 2 - protein, 609 aa, Homo sapiens
MSGWESYYKTEGDEEAEEEQEENLEASGDYKYSGRDSLIFLVDASKAMFESQSEDELTPFDMSIQCIQSVYISKIISSDRDLLAVVFYGTEKDKNSVNFKNIYVLQELDNPGAKRILELDQFKGQQGQKRFQDMMGHGSDYSLSEVLWVCANLFSDVQFKMSHKRIMLFTNEDNPHGNDSAKASRARTKAGDLRDTGIFLDLMHLKKPGGFDISLFYRDIISIAEDEDLRVHFEESSKLEDLLRKVRAKETRKRALSRLKLKLNKDIVISVGIYNLVQKALKPPPIKLYRETNEPVKTKTRTFNTSTGGLLLPSDTKRSQIYGSRQIILEKEETEELKRFDDPGLMLMGFKPLVLLKKHHYLRPSLFVYPEESLVIGSSTLFSALLIKCLEKEVAALCRYTPRRNIPPYFVALVPQEEELDDQKIQVTPPGFQLVFLPFADDKRKMPFTEKIMATPEQVGKMKAIVEKLRFTYRSDSFENPVLQQHFRNLEALALDLMEPEQAVDLTLPKVEAMNKRLGSLVDEFKELVYPPDYNPEGKVTKRKHDNEGSGSKRPKVEYSEEELKTHISKGTLGKFTVPMLKEACRAYGLKSGLKKQELLEALTKHFQD

SEQ ID NO: 3 - DNA, 2171 bp, Mus muscularis
  CDS (190)...(2016)
agtttagggaacagggggtggaagcgggaaatgcgagtgggcgtgcccgcttgaagggtcgaagggcgtgttgtgcgtgatgacgcaacgtcgctgattgggcgagagacaaggagcccgcgtggacgtggtgcccaccttctgctgtcccagtggcttgcccgcctacggttaagtgagcaaaccaacatgtcagagtgggagtcctactacaaaactgagggcgaggaagaggaagaggaggaggagagccccgacacaggtggagaatataaatattcaggaagagatagcttgatttttctggttgatgcctccagggctatgttcgaatctcagggtgaagatgaactcacaccttttgatatgagcatccagtgtatccagagtgtgtacaccagtaagatcataagcagcgatcgggatctcctggcagtggtgttctatggaaccgagaaagataaaaattcagtgaatttcaaaaatatttatgtcttacaagatttggacaacccaggcgctaagcgagtgctagagctcgaccagtttaagggacaacaggggaagaagcacttccgagacacggttggccatgggtctgactactctttgagtgaagtgctctgggtctgtgccaacctcttcagcgacgtccagctcaagatgagtcacaagaggatcatgctgttcaccaatgaagacgacccccatggccgtgacagtgctaaagccagccgggccaggaccaaagccagcgacctccgggacactgggatcttccttgacttgatgcatctgaagaagccaggaggctttgatgtatccgtgttctacagggacatcatcaccaccgctgaggacgaggaccttggggttcacttcgaggagtcaagcaagctggaagacctgctaaggaaggttcgagccaaggagaccaaaaagcgagttctgtccaggttaaagtttaagctcggtgaagacgtagtactcatggtgggcatttataacttggtccagaaagctaacaagccttttccagtgagactctatcgggaaacaaatgaaccagtgaaaaccaagacaaggacttttaatgtaaacaccggcagtctactcctgcctagtgacaccaagcggtctctgacttacgggacacgtcagattgtgctggagaaagaggagacagaggagctgaagcggtttgatgagccaggtttgatcctcatgggctttaagcccacggtgatgctgaagaagcagcactacctgaggccctctctgttcgtgtacccagaggagtccctggtcagtgggagctcaaccttgttcagcgctctgctcaccaagtgtgtggagaagaaggtcatagcagtgtgtagatacacaccccggaagaacgtctccccgtattttgtggctttggtgccccaggaagaggagctggatgatcagaacattcaggtgactccaggaggcttccagcttgtcttcctcccttatgccgatgacaagcggaaggtgccctttactgagaaggtgacggccaaccaggagcagatagacaagatgaaggccattgttcaaaacgtccgcttcacatacaggagcgacagttttgagaatccagtcctgcagcagcacttccgcaacctggaggccctagctttggacatgatggagtcggagcaagtggtagatctgacactacccaaggttgaagccataaagaaaagactgggttccctggcagatgagtttaaagaacttgtctatcctccaggttataatcccgagggaaaagttgccaagagaaaacaagatgatgaaggttctacgagtaaaaagcccaaggtagagttatcagaagaagagctgaaggcccattttcgtaagggcacactgggtaagctcactgtacctacactgaaggacatatgcaaggctcatgggcttaagagtgggccgaagaagcaggaactgctagatgctcttatcagacacttggagaagaactgactaaagctcagaagcccagccactttctacactgcagccaggccacctgcttgtgttcttaccaagctaggtggtagctacaaagagctacttaagtgcaaaatcatggagattacagtctcccacctcactgtgccttactctgtgaataaagc

SEQ ID NO: 4 - protein, 608 aa, Mus muscularis
MSEWESYYKTEGEEEEEEEESPDTGGEYKYSGRDSLIFLVDASRAMFESQGEDELTPFDMSIQCIQSVYTSKIISSDRDLLAVVFYGTEKDKNSVNFKNIYVLQDLDNPGAKRVLELDQFKGQQGKKHFRDTVGHGSDYSLSEVLWVCANLFSDVQLKMSHKRIMLFTNEDDPHGRDSAKASRARTKASDLRDTGIFLDLMHLKKPGGFDVSVFYRDIITTAEDEDLGVHFEESSKLEDLLRKVRAKETKKRVLSRLKFKLGEDVVLMVGIYNLVQKANKPFPVRLYRETNEPVKTKTRTFNVNTGSLLLPSDTKRSLTYGTRQIVLEKEETEELKRFDEPGLILMGFKPTVMLKKQHYLRPSLFVYPEESLVSGSSTLFSALLTKCVEKKVIAVCRYTPRKNVSPYFVALVPQEEELDDQNIQVTPGGFQLVFLPYADDKRKVPFTEKVTANQEQIDKMKAIVQNVRFTYRSDSFENPVLQQHFRNLEALALDMMESEQVVDLTLPKVEAIKKRLGSLADEFKELVYPPGYNPEGKVAKRKQDDEGSTSKKPKVELSEEELKAHFRKGTLGKLTVPTLKDICKAHGLKSGPKKQELLDALIRHLEKN

SEQ ID NO: 5 - DNA, 2372 bp, Sus scrofa
  CDS (235)...(2058)
agttgtgttctgtgtttgttgctcgctggttggggctggcttcgtcctgctttgcccactcagccttgcgcatcccagtagcctctccgcgggacagtgtcagccagaagtgtggggactcgaggcgtggagacagcttctctgattccctttccttcctcccggggggagacagcacccggtgttggaggtccacttgcgtttgagaaacctcactagtgagcagtaaccaacatgtcagggtgggagtcttattacaaaaaccagggcgatgaagaagaagaagaggaggagggccgtgaaacgggtgaagattatagatgttcaggaagagacagtttgatttttttggttgatgcttctagagccatgtttgaatctcaggatgaagatgaattgactccttttgacatgagcatccagtgtatccagagcgtgtacaccaataagatcataagcagtgatcgcgatctcttggcagtggtgttctatggtaccaagaaggacaaaaactcagtgaatttcaaaaatatctacgtcctacaggaattggatagtccaggtgctaaacgagtgcaagagctggatcggtttaaggggcaggaggggacgaagtatttccaagacctgcttggccatggatctgactactcactaagtgaggtgctgtgggtctgcgccaacctcttcagtgacgtccaggtcaagatgagccataagaggatcatgcttttcaccaatgacgacgacccccacggcaacgacagtgccaaagccagccgggccaggaccaaagctggggacctccgtgacacaggtatcttcctggacttgatgcacctgaagaagcctgggggctttgacatatccttgttctacagagatatcatcagtgtggcagaggatgaggacataggggttcactttgaggagtcgcacaagctcgaggacctgttgaggaaggtccgtgccaaggagaccaggaagcgtgtgctccacaggttgaaacttaagctcagcaaagatgtagcgctcactgtcggcatttacaatttggtccagaaggcttacaaacctttcccggtgaggctttatcgggaaacaaatgaaccggtgaaaaccaagacccggacgtttaatgtaaacacaggcagtttgcttttgcctagcgacaccaagaggtctcagacctatgggaatcgtcagattgtattagagaaagaagaaacagaaatgctgaaacggttcgatgaaccgggtttgattctcatcggtttcaagcccttgggaatgctgaagaggcatcattacgtgaggccctcgctgttcgtgtaccccgaggagtccctggtgaatgggagctcaaccctgttcactgctctgctcaccaagtgtctggagaaggaggtcatggcagtgtgcagatatacgccccgcaagaacctccccccgtattttgtggccctgatgccacaggaagaggagctggatgaccagaaagttcaggtgactcccccaggcttccagctcgtcttcttgccctatgctgatgacaaacggaaggtgccctttactgaaaaaatcatggcaaacccagagcaggtggacaagatgaaggccattgttcagaagctccgctttaagtacagaagtgacagctttgagaacccagtgctgcagcaacacttcaggaacatggaggccttggctttggatttgatggagcctgaacaagcagtggatctgacattgcccaaggctgaggcaatggctaaaaggctgggctccctggtggatgagtttaaggaactggtctacccaccagattacaatcccgagggaaaggctcctaagcgaaaacaagacaatgaaagctgtggaagcaaaaggcccaaggtggagttatctgaagaggagctgaaggcccacatcagcgagggcactctagggaagctcactgtgcccaccctgaaggaagcctgcagggtacatggggtgaagggcgggatgaagaagcaggagctgctggatgctctcaccaagcacttccaggactgaccggagaccctacagccagtcacccgtccacgctgtagccagcctgcccgctttcgtttttctcaagttaaatcatgtttctcctgagccaggaagagtacctggcagaagccaaggactgttactttatgagacttcctgttgccatggagacagcgtagccctccccctttgctgtgccttactctgctgtctgaataaagaatcctaactttgaaaaaaaaaaaaaaaaaaaaaaggggggggccgttttaaaaatttccttgggggggccaaagttaacccgaccccattttttttgaaaaaaggggccc

SEQ ID NO: 6 - protein, 607 aa, Sus scrofa
MSGWESYYKNQGDEEEEEEEGRETGEDYRCSGRDSLIFLVDASRAMFESQDEDELTPFDMSIQCIQSVYTNKIISSDRDLLAVVFYGTKKDKNSVNFKNIYVLQELDSPGAKRVQELDRFKGQEGTKYFQDLLGHGSDYSLSEVLWVCANLFSDVQVKMSHKRIMLFTNDDDPHGNDSAKASRARTKAGDLRDTGIFLDLMHLKKPGGFDISLFYRDIISVAEDEDIGVHFEESHKLEDLLRKVRAKETRKRVLHRLKLKLSKDVALTVGIYNLVQKAYKPFPVRLYRETNEPVKTKTRTFNVNTGSLLLPSDTKRSQTYGNRQIVLEKEETEMLKRFDEPGLILIGFKPLGMLKRHHYVRPSLFVYPEESLVNGSSTLFTALLTKCLEKEVMAVCRYTPRKNLPPYFVALMPQEEELDDQKVQVTPPGFQLVFLPYADDKRKVPFTEKIMANPEQVDKMKAIVQKLRFKYRSDSFENPVLQQHFRNMEALALDLMEPEQAVDLTLPKAEAMAKRLGSLVDEFKELVYPPDYNPEGKAPKRKQDNESCGSKRPKVELSEEELKAHISEGTLGKLTVPTLKEACRVHGVKGGMKKQELLDALTKHFQD

SEQ ID NO: 7 - DNA, 1660 bp, Homo sapiens
  CDS (129)...(1133)
cgggatttagatcacgtcccgcaggccggcggaagtagctgatactctcattggttgcaaaaccttgatctgtgaaagcgggcgttttggaagataccggaagtagagtcacggagaggtattaagaaatggagagaaaaataagcagaatccaccttgtttctgaacccagtataactcattttctacaagtatcttgggagaaaacactggaatctggttttgttattacacttactgatggtcattcagcatggactgggacagtttctgaatcagagatttcccaagaagctgatgacatggcaatggaaaaagggaaatatgttggtgaactgagaaaagcattgttgtcaggagcaggaccagctgatgtatacacgtttaatttttctaaagagtcttgttatttcttctttgagaaaaacctgaaagatgtctcattcagacttggttccttcaacctagagaaagttgaaaacccagctgaagtcattagagaacttatttgttattgcttggacaccattgcagaaaatcaagccaaaaatgagcacctgcagaaagaaaatgaaaggcttctgagagattggaatgatgttcaaggacgatttgaaaaatgtgtgagtgctaaggaagctttggagactgatctttataagcggtttattctggtgttgaatgagaagaaaacaaaaatcagaagtttgcataataaattattaaatgcagctcaagaacgagaaaaggacatcaaacaagaaggggaaactgcaatctgttctgaaatgactgctgaccgagatccagtctatgatgagagtactgatgaggaaagtgaaaaccaaactgatctctctgggttggcttcagctgctgtaagtaaagatgattccattatttcaagtcttgatgtcactgatattgcaccaagtagaaaaaggagacagcgaatgcaaagaaatcttgggacagaacctaaaatggctcctcaggagaatcagcttcaagaaaaggaaaagcctgattcttcactacctgagacgtcgaaaaaggagcacatctcagctgaaaacatgtctttagaaactctgagaaacagcagcccagaagacctctttgatgagatttaacagtctcaaaaaatactttgatgttcactagactatgttttctattcatttctttaaaatgaaaaaggagaatttcaagtcagcagccgctattaccgtatcttacaatttaattacatacacagtgaattgaaaccattgtgcaaaatggattacacatgtatacaaagatacgatttgatgatgacactggcacattgagttctaaactattcattcagcatgcctataattacataaattgtatgagactttttgttgcaaaggacacatttatcatattcattcacacatattatatgtgatagctgtccaacatcctgtctgggaagattttgaaaacaggacaaagaaaacatcattttaaaatgtcttcagctttttttgaatagacgtattcaaacatattctgaacattgatgtttgaacattttaatttgtgtgatgatgtagaaaatataattttagtttgtacataaacattgtgaaaatctgataataaaatttttgatacattgaaaaaaaaa

SEQ ID NO: 8 - protein, 334 aa, Homo sapiens
MERKISRIHLVSEPSITHFLQVSWEKTLESGFVITLTDGHSAWTGTVSESEISQEADDMAMEKGKYVGELRKALLSGAGPADVYTFNFSKESCYFFFEKNLKDVSFRLGSFNLEKVENPAEVIRELICYCLDTIAENQAKNEHLQKENERLLRDWNDVQGRFEKCVSAKEALETDLYKRFILVLNEKKTKIRSLHNKLLNAAQEREKDIKQEGETAICSEMTADRDPVYDESTDEESENQTDLSGLASAAVSKDDSIISSLDVTDIAPSRKRRQRMQRNLGTEPKMAPQENQLQEKEKPDSSLPETSKKEHISAENMSLETLRNSSPEDLFDEI

SEQ ID NO: 9 - DNA, 1457 bp, Mus muscularis
  CDS (40)...(1020)
ggagaagaccggaagtggagtactgagaggtagccagaaatggaaaggaaagtaagcagaatctatcttgcttctgaacccaacgtaccttattttctgcaagtgtcttgggagagaacaataggatccggctttgttattacacttactgacggccattcagcctggactgcaacagtttctgaattggagatttcccaagaagctgatgacatggctatggagaaaggaaaatacattgatgagctgagaaaggcactggtgccagggtcaggagcagctggtacatacaagttcctcttttctaaggagtctcggcatttctcccttgagaaagaactgaaagatgtttcattcagacttggttccttcaacctagacaaagtttcaaactcagctgaagtcataagagatctcatttgttactgcctggacaccattacagaaaagcaagccaaaaacgagcacctgcagaaagaaaatgaaaggctcctgagagactggaatgatgttcaaggccgatttgagaaatgtgtgagtgccaaagaagccttggaggctgatttgtatcaaagatttatcttggtgctgaatgagaagaaaacaaagatccggagcttgcataaattgctaaatgaagtccagcagctggaggagagtaccaaacctgaaagggaaaatccgtgttctgacaaaacacctgaggagcatgggctttatgatggaagcaccgatgaagaaagtggagcccctgtgcaggctgcagaaactcttcataaggatgattccatattttcaagtccggatgtcactgacattgcaccaagtaggaagaggagacaccgaatgcagaagaatcttgggacagaacctaaaatggctccacaggagttgccgctacaggagaaggaaaggcttgcctcttcactaccccagaccttgaaagaggagagcacctctgctgaaaacatgtctttagaaaccctgagaaacagcagcccagaagatctctttgattagatataaaccctagaaaacagcactggtgttcaatgaactatattctttatccagttcctttggatgagaagacaggctttcaacttagcagccatggttactatgtcttgcaactttactatatgtacccaatgaatcatactagtatgtgacagtcagatggaatgtcagagggacacttttctccctttcttaattaaatctgagttttgttgcaaaaggcacatatatcatatttgtttatacatttttgtaactttctaattgactcttggaagattttggaaatagaacaaaatatctttaacacttttttagttgcactttaaattatctttgttgaagcatttaatttatgatgtgacacatataaaatacatctttatcttgtgtgtaaacagtctgaaatctgatagttaataaaagtttgtgttccc

SEQ ID NO: 10 - protein, 326 aa, Mus muscularis
MERKVSRIYLASEPNVPYFLQVSWERTIGSGFVITLTDGHSAWTATVSELEISQEADDMAMEKGKYIDELRKALVPGSGAAGTYKFLFSKESRHFSLEKELKDVSFRLGSFNLDKVSNSAEVIRDLICYCLDTITEKQAKNEHLQKENERLLRDWNDVQGRFEKCVSAKEALEADLYQRFILVLNEKKTKIRSLHKLLNEVQQLEESTKPERENPCSDKTPEEHGLYDGSTDEESGAPVQAAETLHKDDSIFSSPDVTDIAPSRKRRHRMQKNLGTEPKMAPQELPLQEKERLASSLPQTLKEESTSAENMSLETLRNSSPEDLFD

SEQ ID NO: 11 - DNA, 1609 bp, Sus scrofa
  CDS (71)...(1072)
aaaagttctttacaactccgcagcgcacagatttttcccggcttggattcccctcgatgtgtgaccagaaatggagagaaaagtaagcagaatctatctcacttctgaacctgaaataattcattttctacaagtcgcttgggagaaaacactaggatctggttttgttattacccttactgatgggcagtcagcatggattgggacagtttctgaatcagagatttcccaagaagctgatgacatggcattggaaaaagagaaatatgttgatgaactgagaaaagcattggtgtcaggagcaggaccaactgacacatacaagtttaatttttcaaaagactcttgtcaattctcctttgagaagaacttgaaagatgtttcattcagacttggttccttcaacttagagaaagttgcaaacccagctgaggtcattagagaacttatttgttactgcctggacaccatctcagaaaagcaagccaaaaatgagcacctgcagaaagaaaatgaaaggcttctgagagattggaatgacgttcaagaacgatttgaaaaatgtgttagtgctaaggaagctgtggagactgatctttataaacgatttattctggtgctgaatgagaagaaagcaaagatcagaagcttatataaattgttaaatgaagttcaagaacttgaaaagaacatcgagcataaaagggaaattccaacatgttctgaaatgactactgacagagattcaatctatgatgaaagtaccgatgaggaaaatgaaattccacctaatccctctgtattaacttcagctactttaaaaagagatgattccattatttcaagtcctgatgtcactgatattgcaccgagtagaaagagaaggcagcggatgcaaaaaaatcttgggacagaacctaaaatggcttcacaggagcttcagcttcaagaaaaagaaaagcttgatcctccactacctgagaagctgaaaaaggatcacagcccagctgaaaacatgtctttagaaactctaaaaaacagtagtccaggagacctctttgatgagatttaattgtctcagagaactactttgatgttcactagactgtgttttccaatcatttcctttaaattgaaaaagaaaaatttcaactcagcagcagctattactgtaccttacaatttaattacatacacactgagttgaaatcattgtgcaaaatggattacacatgtatataaagatattatttgatgacagtggtgcattattctaaagtactcattcagcatgcctataattatataaaatctcagaccttttttttttttttttaacaaaggtcacatttaccttattcaattcacatatattacgtatggtagctatctaacatcctgtctaggaagatattggaaaaagtacaaagaacatgtctccaggttcttctttgaataatcactttctgagcaataatgcttgaacattttaacttgtggaatgatgtgtaaaatctatttttagtttggacacaacccaaaaatcttatgataaaatttttgattaaagatttttttgttctcaataaaatatattaattagtaaaaa

SEQ ID NO: 12 - protein, 333 aa, Sus scrofa
MERKVSRIYLTSEPEIIHFLQVAWEKTLGSGFVITLTDGQSAWIGTVSESEISQEADDMALEKEKYVDELRKALVSGAGPTDTYKFNFSKDSCQFSFEKNLKDVSFRLGSFNLEKVANPAEVIRELICYCLDTISEKQAKNEHLQKENERLLRDWNDVQERFEKCVSAKEAVETDLYKRFILVLNEKKAKIRSLYKLLNEVQELEKNIEHKREIPTCSEMTTDRDSIYDESTDEENEIPPNPSVLTSATLKRDDSIISSPDVTDIAPSRKRRQRMQKNLGTEPKMASQELQLQEKEKLDPPLPEKLKKDHSPAENMSLETLKNSSPGDLFDEI

SEQ ID NO: 13 - DNA, 2254 bp, Homo sapiens
  CDS (258)...(1277)
gggaattctgaaagccgctggcggaccgcgcgcagcggccagagaccgagccctaaggagagtgcggcgcttcccgaggcgtgcagctgggaactgcaactcatctgggttgtgcgcagaaggctggggcaagcgagtagagaagtggagcgtaagccaggggcgttgggggccgtgcgggtcgggcgcgtgccacgcccgcggggtgaagtcggagcgcggggcctgctggagagaggagcgctgcggaccgagtaatggcaatgcagatgcagcttgaagcaaatgcagatacttcagtggaagaagaaagctttggcccacaacccatttcacggttagagcagtgtggcataaatgccaacgatgtgaagaaattggaagaagctggattccatactgtggaggctgttgcctatgcgccaaagaaggagctaataaatattaagggaattagtgaagccaaagctgataaaattctggctgaggcagctaaattagttccaatgggtttcaccactgcaactgaattccaccaaaggcggtcagagatcatacagattactactggctccaaagagcttgacaaactacttcaaggtggaattgagactggatctatcacagaaatgtttggagaattccgaactgggaagacccagatctgtcatacgctagctgtcacctgccagcttcccattgaccggggtggaggtgaaggaaaggccatgtacattgacactgagggtacctttaggccagaacggctgctggcagtggctgagaggtatggtctctctggcagtgatgtcctggataatgtagcatatgctcgagcgttcaacacagaccaccagacccagctcctttatcaagcatcagccatgatggtagaatctaggtatgcactgcttattgtagacagtgccaccgccctttacagaacagactactcgggtcgaggtgagctttcagccaggcagatgcacttggccaggtttctgcggatgcttctgcgactcgctgatgagtttggtgtagcagtggtaatcactaatcaggtggtagctcaagtggatggagcagcgatgtttgctgctgatcccaaaaaacctattggaggaaatatcatcgcccatgcatcaacaaccagattgtatctgaggaaaggaagaggggaaaccagaatctgcaaaatctacgactctccctgtcttcctgaagctgaagctatgttcgccattaatgcagatggagtgggagatgccaaagactgaatcattgggtttttcctctgttaaaaaccttaagtgctgcagcctaatgagagtgcactgctccctggggttctctacaggcctcttcctgttgtgactgccaggataaagcttccgggaaaacagctattatatcagcttttctgatggtataaacaggagacaggtcagtagtcacaaactgatctaaaatgtttattccttctgtagtgtattaatctctgtgtgttttctttggttttggaggaggggtatgaagtatctttgacatggtgccttaggaatgacttgggtttaacaagctgtctactggacaatcttatgtttccaagagaactaaagctggagagacctgacccttctctcacttctaaattaatggtaaaataaaatgcctcagctatgtagcaaagggaatgggtctgcacagattctttttttctgtcagtaaaactctcaagcaggtttttaagttgtctgtctgaatgatcttgtgtaagggtttggttatggagtcttgtgccaaacctactaggccattagcccttcaccatctacctgcttggtctttcattgctaagactaactcaagataatcctagagtcttaaagcatttcaggccagtgtggtgtcttgcgcctgtactcccagcactttgggaggccgaggcaggtggatcgcttgagccaggagttttaagtccagcttggccaagatggtgaaatcccatctctacaaaaaatgcagaacttaatctggacacactgttacacgtgcctgtagtcccagctactctatagcctgaggtgggagaatcacttaagcctggaaggtggaagttgcagtgagtcgagattgcactgctgcattccagccagggtgacagagtgagaccatgtttcaaacaagaaacatttcagagggcaagtaaacagatttgattgtgaggcttctaataaagtagttattagtagtg

SEQ ID NO: 14 - protein, 339 aa, Homo sapiens
MAMQMQLEANADTSVEEESFGPQPISRLEQCGINANDVKKLEEAGFHTVEAVAYAPKKELINIKGISEAKADKILAEAAKLVPMGFTTATEFHQRRSEIIQITTGSKELDKLLQGGIETGSITEMFGEFRTGKTQICHTLAVTCQLPIDRGGGEGKAMYIDTEGTFRPERLLAVAERYGLSGSDVLDNVAYARAFNTDHQTQLLYQASAMMVESRYALLIVDSATALYRTDYSGRGELSARQMHLARFLRMLLRLADEFGVAVVITNQVVAQVDGAAMFAADPKKPIGGNIIAHASTTRLYLRKGRGETRICKIYDSPCLPEAEAMFAINADGVGDAKD

SEQ ID NO: 15 - DNA, 1020 bp, Mus muscularis
  CDS (1)...(1020)
atggctatgcaaatgcagcttgaagcaagtgcagatacttcagtggaagaagaaagttttggtccacagcctatttcacggttagagcagtgtggcataaatgccaatgatgtgaagaaattagaagaagccggttaccatacagtggaggctgttgcttatgcaccgaagaaggaactaataaatattaagggaattagtgaagccaaagctgacaaaattctgactgaggcagcaaaattggttccaatgggtttcaccacggcaactgagtttcaccagcgccggtcagagatcatacagataactactggctccaaagagctggacaagctgcttcaaggtggaattgagactggatctatcacagagatgtttggagaattccgaactgggaagacacagatctgtcatacgttggctgttacatgccagctccccattgaccgtggtggaggtgaagggaaggccatgtacattgacaccgagggcacctttaggccggagcggctgctagcagtagctgagagatacggtctctctggcagcgatgtcctagataatgtagcatatgcgcgagggttcaacacagaccaccagacccagctcctttaccaagcgtcagccatgatggtagaatccaggtatgcactgcttattgtagacagtgctactgccctttacagaacagactactcagggcggggagagctttcagccaggcaaatgcatttggccagatttctgaggatgctgcttcgacttgctgatgagtttggtgtcgcagtggtaatcaccaaccaggtagtagcccaagtagatggagcagccatgttcgctgcagatcccaaaaaacccattggagggaacatcatcgctcatgcgtcaaccaccaggctgtacctgagaaaaggaagaggggagaccagaatctgcaaaatctatgactctccctgtcttcctgaagctgaagctatgtttgccattaatgcagatggagtgggcgatgccaaagactga

SEQ ID NO: 16 - protein, 339 aa, Mus muscularis
MAMQMQLEASADTSVEEESFGPQPISRLEQCGINANDVKKLEEAGYHTVEAVAYAPKKELINIKGISEAKADKILTEAAKLVPMGFTTATEFHQRRSEIIQITTGSKELDKLLQGGIETGSITEMFGEFRTGKTQICHTLAVTCQLPIDRGGGEGKAMYIDTEGTFRPERLLAVAERYGLSGSDVLDNVAYARGFNTDHQTQLLYQASAMMVESRYALLIVDSATALYRTDYSGRGELSARQMHLARFLRMLLRLADEFGVAVVITNQVVAQVDGAAMFAADPKKPIGGNIIAHASTTRLYLRKGRGETRICKIYDSPCLPEAEAMFAINADGVGDAKD

SEQ ID NO: 17 - DNA, 1134 bp, Sus scrofa
  CDS (239)...(1134)
ctgcaggttcggcgcgcgctgcggcctgagaccgaggtctgaggagagctcagacgcgcagctgggagctgagaagtttcgtgtattccgtgccgaaggccggagcaggggagtagagaggtggacgctgcgaggggctctgcactccgtccggggtcgagcgcgcgcgggtccagcggactgaggttcggaacgctcagccggccagagcgggtagcgctgcaggccgagtattgaacactgagtaatggctgtgcagatgcagcttgaatcaaatgcagatacttcggtggaagaggagagctttggcccacagcctatttcacgattagagcaatgtggcataaatgccaatgatgtcaaaaaattggaagaagctggattccacaccgtggaggctgttgcctatgcaccaaagaaggagctaataaatattaaggggattagtgaagccaaagctgataaaattctgaccgaggcagctaaattagttccaatgggtttcaccactgctactgagttccaccaaaggcgatccgagatcatacaaattactactggctccaaagagcttgacaagctacttcaaggaggaattgagactggatccatcacagagatgtttggagaattccgaactgggaaaacccagatctgtcatacattggctgtaacatgccagcttcccatcgaccgaggtggaggtgaaggaaaggccatgtacattgacactgagggtaccttcaggccagaacggctgctagcagtggctgagagatacggcctctctggcagtgatgtcctggataacgtagcatatgcccgggcgttcaacacagaccaccagacccagctcctttatcaagcatcagccatgatggtagagtccaggtatgcactgctaattatagacagtgccaccgccctctacagaacggactattcgggtcgaggtgagctttcagccaggcagatgcacttggccaggtttctgcggatgcttctgcgacttgctgatgagtttggtgtagcagtggtaatcaccaaccaggtggtagcccaagtggatggagcagccatgtttgctgcagatcctaaaaaacctattggaggaaatatcattgctcatgcctc

SEQ ID NO: 18 - protein, 297 aa, Sus scrofa
HVMAVQMQLESNADTSVEEESFGPQPISRLEQCGINANDVKKLEEAGFHTVEAVAYAPKKELINIKGISEAKADKILTEAAKLVPMGFTTATEFHQRRSEIIQITTGSKELDKLLQGGIETGSITEMFGEFRTGKTQICHTLAVTCQLPIDRGGGEGKAMYIDTEGTFRPERLLAVAERYGLSGSDVLDNVAYARAFNTDHQTQLLYQASAMMVESRYALLIIDSATALYRTDYSGRGELSARQMHLARFLRMLLRLADEFGVAVVITNQVVAQVDGAAMFAADPKKPIGGNIIAHA

SEQ ID NO: 19 - DNA, 3107 bp, Homo sapiens
  CDS (662)...(2905)
cggtgagtcttggcggctgttaacgcgcgctttgggaacaggaaggttgagagagaggtgctggggtctgcgtctatctctgtcgctcttttcagcccctcctggtattcccctcctaacctgggttttttacacgcccgcgtggcttcctgctcgacctccctgagtctgatcctggtttccacctccagccctgggaaatttcctttctccagactcgccctccccacccgggcctcggactttcaccccagcttctctctcctggccagtgattacccacccccaatcccaccccgccccgccgcgcaactacctcctcccttcacccggactgggaccatcatccccactccactccgcccagtctgggactccacctgcctcctccccaatcccacactaatctctgcttggtctcttcctctttggcctaatctctcgtctcggcttattggggacggccactctcacagtttggttccaaacaccagttcctggatggattcccgccatccatgccccctctttaattagccggtcctctcagtaatgtagcagccccctctacagattagaccctggtcctacactcttagccgctgcctgcttttgacctttggctcatgggtacttgacgttttaaactcctaggcccaggatgaggaggagcttggctcccagccagctggccaagagaaaacctgaaggcaggtcctgtgatgatgaagactggcaacctggcctagtgactcctaggaaacggaaatccagcagtgagacccagatccaggagtgtttcctgtctccttttcggaaacctttgagtcagctaaccaatcaaccaccttgtctggacagcagtcagcatgaagcatttattcgaagcattttgtcaaagcctttcaaagtccccattccaaattatcaaggtcctctgggctctcgagcattgggcctgaaaagggctggggtccgccgggccctccatgaccccctggaaaaagatgccttggttctgtatgagcctcccccgctgagcgctcatgaccagctgaagcttgacaaggagaaactccctgtccatgtggttgttgaccctattctcagtaaggttttgcggcctcatcagagagagggagtgaaattcctgtgggagtgtgtcaccagtcggcgcatccctggcagccatggctgcatcatggctgatgagatgggcctaggaaagacgctgcagtgcatcacattgatgtggacacttttacgccagagtccagagtgcaagccagaaattgacaaggcagtggtggtgtcgccttccagcctggtgaagaactggtacaatgaggttgggaaatggctcggagggaggatccaacctctggccatcgatggaggatctaaggatgaaatagaccaaaagctggaaggattcatgaaccagcgtggagccagggtgtcttctcccatcctcatcatttcctatgagaccttccgccttcatgttggagtcctccagaaaggaagtgttggtctggtcatatgtgacgagggacacaggctcaagaactctgagaatcagacttaccaagccctggacagcttgaacaccagccggcgggtgctcatctccggaactcccatccagaatgatctgcttgagtatttcagcttggtacattttgttaattccggcatcctagggactgcccatgaattcaagaagcattttgaattgccaattttgaagggtcgagacgctgctgctagtgaggcagacaggcagctaggagaggagcggctgcgggagctcaccagcattgtgaatagatgcctgatacggaggacttctgatatcctttctaaatatctgcctgtgaagattgagcaggtcgtttgttgtaggctgacaccccttcagactgagttatacaagaggtttctgagacaagccaaaccggcagaagaattgcttgagggcaagatgagtgtgtcttccctttcttccatcacctcgctaaagaagctttgtaatcatccagctctaatctatgataagtgtgtggaagaggaggatggctttgtgggtgccttggacctcttccctcctggttacagctctaaggccctggagccccagctgtcaggtaagatgctggtcctggattatattctggcggtgacccgaagccgtagcagtgacaaagtagtgctggtgtcgaattacacccagactttggatctctttgagaagctgtgccgtgcccgaaggtacttatacgtccgcctggatggcacgatgtccattaagaagcgagccaaggttgtagaacgcttcaatagtccatcgagccctgactttgtcttcatgctgagcagcaaagctgggggctgtggcctcaatctcattggggctaaccggctggtcatgtttgaccctgactggaacccagccaatgatgaacaagccatggcccgggtctggcgagatggtcaaaagaagacttgctatatctaccgcctgctgtctgcagggaccattgaggagaagatcttccagcgtcagagccacaagaaggcactgagcagctgtgtggtggatgaggagcaggatgtagagcgccacttctctctgggcgagttgaaggagctgtttatcctggatgaagctagcctcagtgacacacatgacaggttgcactgccgacgttgtgtcaacagccgtcagatccggccaccccctgatggttctgactgcacttcagacctggcagggtggaaccactgcactgataagtgggggctccgggatgaggtactccaggctgcctgggatgctgcctccactgctatcaccttcgtcttccaccagcattctcatgaggaacagcggggcctccgctgataaccagctggtctgggtgtagctcttagaggaaggagatagggaaaaggggctccttgctccacagggccctgttgaattttgttctctgggagaaaatcatcaagaagggctgcatgatgtttgcccaaaatttattttataagaaaaacttttttggttaaaaaaaagaataaaggtatgaaagggttaaaaaaaaaaa

SEQ ID NO: 20 - protein, 747 aa, Homo sapiens
MRRSLAPSQLAKRKPEGRSCDDEDWQPGLVTPRKRKSSSETQIQECFLSPFRKPLSQLTNQPPCLDSSQHEAFIRSILSKPFKVPIPNYQGPLGSRALGLKRAGVRRALHDPLEKDALVLYEPPPLSAHDQLKLDKEKLPVHVVVDPILSKVLRPHQREGVKFLWECVTSRRIPGSHGCIMADEMGLGKTLQCITLMWTLLRQSPECKPEIDKAVVVSPSSLVKNWYNEVGKWLGGRIQPLAIDGGSKDEIDQKLEGFMNQRGARVSSPILIISYETFRLHVGVLQKGSVGLVICDEGHRLKNSENQTYQALDSLNTSRRVLISGTPIQNDLLEYFSLVHFVNSGILGTAHEFKKHFELPILKGRDAAASEADRQLGEERLRELTSIVNRCLIRRTSDILSKYLPVKIEQVVCCRLTPLQTELYKRFLRQAKPAEELLEGKMSVSSLSSITSLKKLCNHPALIYDKCVEEEDGFVGALDLFPPGYSSKALEPQLSGKMLVLDYILAVTRSRSSDKVVLVSNYTQTLDLFEKLCRARRYLYVRLDGTMSIKKRAKVVERFNSPSSPDFVFMLSSKAGGCGLNLIGANRLVMFDPDWNPANDEQAMARVWRDGQKKTCYIYRLLSAGTIEEKIFQRQSHKKALSSCVVDEEQDVERHFSLGELKELFILDEASLSDTHDRLHCRRCVNSRQIRPPPDGSDCTSDLAGWNHCTDKWGLRDEVLQAAWDAASTAITFVFHQHSHEEQRGLR

SEQ ID NO: 21 - DNA, 2244 bp, Mus muscularis
  CDS (1)...(2244)
atgaggaggagcttagctcccagccagttggctaggaggaaaccagaagacagatcatcagatgatgaagactggcagcctgggacagtaactcctaagaaacgcaagtccagcagtgagacccaggtccaggagtgtttcctgtctccttttcggaagcccttgactcagctactcaaccggccaccttgtctggatagcagtcaacatgaagcatttattcgaagtattttgtcaaagcctttcaaggttcccatcccaaattatcaaggtcccctgggctctcgtgcattgggcctgaaaagggctggagttcgtcgtgcccttcatgaccctctggaagaaggtgccttggttctctatgagcctcccccactcagcgcccatgaccaactgaagcttgacaaggaaaaactccctgttcacgtggttgttgatcctgttctcagtaaggtattgcggcctcatcagagagagggagtgaagttcctatgggagtgtgtcaccagtcgtcgaattcctggaagccatggctgcatcatggctgatgagatgggcctggggaagacactgcagtgcatcacattgatgtggacacttttacgccagagcccggaatgcaagccagaaatcgagaaggcagtggtggtgtcaccttctagcctggtgaagaactggtacaatgaggttgagaaatggcttggagggaggattcaacctctggccatcgatggaggctcgaaggacgagatagaccgaaaactggaaggattcatgaaccagcgtggagctagagtgccttcccccatcctcatcatttcctatgagactttccgcctgcatgttggagttcttaaaaaaggaaatgttggactggtcatatgtgacgagggccacaggctaaagaactctgagaatcagacttaccaggctctggacagcttgaataccagccggcgggtgctaatctccgggacccccatccaaaatgatttgctggagtatttcagcttggtgcactttgtaaattctggcattttgggaactgcccatgaattcaagaagcattttgagttgccaattttgaagagtcgagatgcagctgccagtgaggcagacaggcagcgtggggaggagcgtctgcgggagctcatcggtattgtgaacaggtgcctgatacggagaacatctgatatcctctctaagtatctgcccgtgaagattgagcaggtggtttgttgtaggctgacaccccttcaaactgagctatacaagagatttctgagacaggctaagcctgaagaagaattgcgtgagggcaagatgagtgtgtcttccctgtcttctatcacctctctaaagaagctgtgtaatcatccagctctaatctatgacaagtgtgtggcagaggaggatggctttgagggcactttgggtatcttcccacctggttataactctaaagctgtagagccacagttgtcaggtaagatgctggtccttgattacattctggccgtgactcgaagccgtagcagtgacaaagtcgtgctggtgtctaattatactcagacattggatctctttgaaaagctgtgccgggttcgaaggtacttgtatgttcgcctggatggcacgatgtccattaagaagcgagccaaggttgtggagcgcttcaatagcccatcgagccctgattttgttttcatgctgagcagcaaagccgggggctgtggtcttaacctcattggtgctaaccgactggtcatgtttgatcctgactggaatccagccaatgatgaacaagctatggcccgagtctggcgtgatggtcaaaagaagatctgctatatctaccgactactatctgcaggaacaattgaggagaagatctttcagcggcagagccacaagaaggctctgagcagctgtgtggtggacgaggagcaggatgtggagcgccacttttctcttggtgagcttaaagagctgtttactctggatgaagcaagcctcagtgacacacatgacagattgcattgccgccgttgtgtaaacaaccgtcaggtctggccaccccctgatggttctgactgcacttcagacctggctcagtggaaccacagcacagataaacgaggcctccaggatgaggtactccaggctgcctgggatgcttcatctacagccatcaccttcgtcttccaccagcgttctcatgaggagcagcgcggtcttcactga

SEQ ID NO: 22 - protein, 747 aa, Mus muscularis
MRRSLAPSQLARRKPEDRSSDDEDWQPGTVTPKKRKSSSETQVQECFLSPFRKPLTQLLNRPPCLDSSQHEAFIRSILSKPFKVPIPNYQGPLGSRALGLKRAGVRRALHDPLEEGALVLYEPPPLSAHDQLKLDKEKLPVHVVVDPVLSKVLRPHQREGVKFLWECVTSRRIPGSHGCIMADEMGLGKTLQCITLMWTLLRQSPECKPEIEKAVVVSPSSLVKNWYNEVEKWLGGRIQPLAIDGGSKDEIDRKLEGFMNQRGARVPSPILIISYETFRLHVGVLKKGNVGLVICDEGHRLKNSENQTYQALDSLNTSRRVLISGTPIQNDLLEYFSLVHFVNSGILGTAHEFKKHFELPILKSRDAAASEADRQRGEERLRELIGIVNRCLIRRTSDILSKYLPVKIEQVVCCRLTPLQTELYKRFLRQAKPEEELREGKMSVSSLSSITSLKKLCNHPALIYDKCVAEEDGFEGTLGIFPPGYNSKAVEPQLSGKMLVLDYILAVTRSRSSDKVVLVSNYTQTLDLFEKLCRVRRYLYVRLDGTMSIKKRAKVVERFNSPSSPDFVFMLSSKAGGCGLNLIGANRLVMFDPDWNPANDEQAMARVWRDGQKKICYIYRLLSAGTIEEKIFQRQSHKKALSSCVVDEEQDVERHFSLGELKELFTLDEASLSDTHDRLHCRRCVNNRQVWPPPDGSDCTSDLAQWNHSTDKRGLQDEVLQAAWDASSTAITFVFHQRSHEEQRGLH

SEQ ID NO: 23 - DNA, 2641 bp, Sus scrofa
gggtgctcatctctgggacccccatccagaacgacctccttgagtatttcagcttggtccattttgttaattcgggcctcctaggaactgcccatgagttcaagaagcgttttgaactgccgattctgaagggtcgagatgcagcggccagtgaggaagaccggcggctgggagaggagcgactgcgggagctcaccagcattgtgaacaggtgtctgatacggaggacatctgatatcctttctaaatacctgcctgtgaagattgagcaggtggtttgttgcaggctgacaccccttcagactgaattatacaagaggtttttgagacaggccaagccagcagaagagttgcgtgagggcaagatgagtgtgtcttctctctcttccatcacgtccctgaagaagctgtgtaaccatccagctctaatctatgacaagtgtgtggaagaggaagatggcttcgcaggtgccttggacttctttccccctggttatagctccaaggctttagagcctcagctgtcaggtaagatgctggtccttgattacattctggcggtgacccgaagccgcagcagtgacaaagtcgtgctggtgtccaattacactcagacattggacctctttgagaaactctgccgggcccgaaggtacctgtatgttcgcttggatggcacaatgtccattaagaagcgagccaaggttgtggagcgcttcaacagcccgtcgagtccagactttgtcttcatgctgagcagcaaagctgggggctgtggcctcaatctcatcggggctaaccgcctggtcatgtttgaccctgactggaacccagccaacgatgaacaagccatggcccgggtctggcgtgacggtcaaaagaagacctgctatatctatcgcctgctgtctgcaggaaccattgaggagaagatctttcagcgtcagagccacaagaaggcactgagcagctgcgtggtggacgaggagcaggatgtggagcggcacttctctctaggcgaactgaaggagctctttaccctggatgaggccagcctcagtgacacacatgacaggttgcgctgccgtcgctgtgtcaacaaccagcaggtgtggccaccccccgacggttctgactgcacctcagacctggctcagtggaaccacagcactgataagcgggggctcgcggatgaggtactccaggctgcctggcacgctgcctccactgccatcaccttcgtctttcaccagcgttcccacgaggagcagcggggcctccgctaataaccagcggctctgggtgtggctgttggaggaaggagaggggagagggggtccctgtcccatggggcacggctgaattttgttctctgagagaaaaatcatccagaagggctgcatgatgtttgcccaaaatttatttttataagaaaaccttttggttaaaaaagaaaaggaaggaataaaggtgtgagagggactgaggcctggggacggcatggtgagcccagcagaggagaggccgggtcgtgggcacttgtgcacgcctctaaagcctggctgcctgcttgaggagggagagcaggaatcccgggcccggggggggctgtcctcagcaacagatgaccactgtaacctcccggcccaaggctcccagggtactggggacgggcccaaaggctctaaacccagttgtgggaatgcctttctttgctgggttccatcatcaaaccctccaggacctcagcgcgagggaagaaggaaaaaagagaaaaacaggtggcagaaagagagagacagaattggtgtttgggagttctgggcagcccatgcctcagcccctgcaagctgatggtactgagcctgggccttggaggcgtgggcccatcacatggtgctgacataatctgcaaaggcttgggatgagtcccacgagtcgctaaccacatggcaggtggcccggagccgtcggatggcttccctggctgtgactgcatcctggtccagccagttctggaagagacgcaggtgaccaaaggctccacggccccagcgctctagccttttggcgaactccagaagcccgtctggcttgatgcagttggaactgatatccagctgacacagggaggaggatgagtcttctgagaagacatctgccagggccagcaggccagcgtttcccaggcggttccctggcagccggaggcccttcagtgtggagttgcccttcatagcagcaaccatctcaggcagaaactgggctgggcgcttctcgaacaggcggcagaaggagaaggtaatctcttgaagagtcaaattctggagcaagaagagcacctcgctctgacagtcagccaggttcatgtcatggaagctcagcctcttcagggcgaggttgtactctttgagtgtctgtaacacaagcccaaagtacagagcagagcagtggggctggctgggctccccgtggaaatcccatctccccccgttccaggcagttttatggtatctcctcatccccaactatgtggctagggggccctgcattccccggcacagcagggttgtccctctggctggggtggtcaacacgaatggagaggacccgtagcaggggcagggctcgctcaatggca

(19) United States
(12) Patent Application Publication
(10) Pub. No.: US 2007/0155014 A1
(43) Pub. Date: Jul. 5, 2007

(54) Methods for increasing efficiency of homologous recombination

(75) Inventors: Luciana R. Bertolini, Davis, CA (US); Michaeline Bunting, San Diego, CA (US); Knut Madden, Carlsbad, CA (US); Elizabeth A. Maga, Davis, CA (US); James D. Murray, Davis, CA (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignees: Invitrogen Corporation, Carlsbad, CA (US); The Regents of The University of California, Oakland, CA (US)

(21) Appl. No.: 11/490,474

(22) Filed: Jul. 20, 2006

Related U.S. Application Data
(60) Provisional application No. 60/701,399, filed on Jul. 20, 2005.
(60) Provisional application No. 60/707,911, filed on Aug. 12, 2005.

(51) Int. Cl. C12N 15/09; C12N 5/10

(52) U.S. Cl. 435/455; 435/325

(57) ABSTRACT

Methods and compositions for increasing or decreasing homologous recombination activity in a eukaryotic cell are provided. More particularly, various methods and compositions are provided which decrease the level of non-homologous recombination in a cell and thereby increase the frequency of targeted homologous recombination events. Various compositions including cells and kits that can be employed in the methods are also provided.

### CROSS-REFERENCE TO RELATED APPLICATIONS

This application claims the benefit of U.S. Provisional Application No. 60/701,399, filed on Jul. 20, 2005, and U.S. Provisional Application No. 60/707,911, filed on Aug. 12, 2005, both of which are herein incorporated by referenced in their entirety.

### FIELD OF THE INVENTION

The field of the invention relates generally to the fields of molecular biology, developmental biology, biochemistry and medicine. Generally, methods, compositions, and kits for initiating, modulating and/or increasing homologous recombination activity in a cell are provided.

### BACKGROUND OF THE INVENTION

While the integration of heterologous DNA into cells and organisms is potentially useful to produce transformed cells and organisms which are capable of expressing desired genes and/or polypeptides, many problems are associated with such systems. A major problem resides in the random pattern of integration of the heterologous gene into the genome of cells derived from multicellular organisms such as mammalian cells. This often results in a wide variation in the level of expression of such heterologous genes among different transformed cells. Further, random integration of heterologous DNA into the genome may disrupt endogenous genes which are necessary for the maturation, differentiation and/or viability of the cells or organism. In the case of transgenic animals, gross abnormalities are often caused by random integration of the transgene and gross rearrangements of the transgene and/or endogenous DNA often occur at the insertion site.

One approach to overcome problems associated with random integration involves targeting the insertion of the transgene into a predetermined position in the genome. This method involves selecting homologous recombination events between DNA sequences residing in the genome of a cell or organism and newly introduced DNA sequences. Such methods provide a means for systematically altering the genome of the cell or organism.

A significant problem encountered in detecting and isolating cells, such as mammalian and plant cells, wherein homologous recombination events have occurred lies in the greater propensity for such cells to mediate non-homologous recombination. Accordingly, methods and compositions are needed in the art that allow for improved homologous recombination efficiency and thereby improving the frequency that a transgene can be integrated in the genome at a predetermined location.

### BRIEF SUMMARY OF THE INVENTION

Methods, compositions, and kits are provided to increase or decrease the homologous recombination activity in a cell.

In one method, the efficiency of homologous recombination of a polynucleotide of interest in a eukaryotic cell is increased. In some aspects, the method comprises providing a eukaryotic cell having a decreased level of non-homologous recombination activity, where the decreased level of non-homologous recombination activity is transient; and, introducing into the eukaryotic cell a composition comprising a homologous recombination cassette, wherein the polynucleotide of interest is inserted into the target site by a homologous recombination event.

In some aspects of the invention, the homologous recombination cassette comprises the polynucleotide of interest flanked by a first region and a second region having sufficient identity to a corresponding second region of the target site.

In some aspects of the invention, the eukaryotic cell comprises at least one polynucleotide comprising a silencing element, wherein the silencing element reduces the level of a gene product that contributes to non-homologous recombination. The gene product that contributes to non-homologous recombination can encode, but is not limited to, a Ku70 polypeptide, a Xrcc4 polypeptide, a Ligase IV polypeptide, a DNA-dependant protein kinase catalytic subunit (DNA-PKcs) polypeptide or an Artemis nuclease polypeptide or any combination thereof.

In some aspects of the invention, the polynucleotide encoding the silencing element is stably incorporated into the cell and is operably linked to an inducible promoter. In other embodiments, the silencing element comprises an siRNA silencing element, an miRNA silencing element, a double stranded RNA silencing element, a hairpin RNA silencing element, protein nucleic acid molecule (PNA molecule), an antisense silencing element or a sense silencing element.

In further other, the target site is endogenous to the eukaryotic cell or the target site is heterologous to the eukaryotic cell. The target site can be chromosomally located or extrachromosomally located in the eukaryotic cell.

In still further aspects, the eukaryotic cell further comprises or the cell is further provided at least one heterologous polynucleotide which when expressed in the eukaryotic cell increases homologous recombination activity in the eukaryotic cell. Such heterologous polynucleotides can encode, but are not limited to, a RecA polypeptide, a Rad54 polypeptide, a Rad51 polypeptide, or any combination thereof. In still further embodiments, the heterologous polynucleotide comprises a silencing element which reduces the level of a helicase polypeptide, a cell-cycle polypeptide, a Structural Maintenance of Chromosomes (SMC) polypeptide, a topoisomerase polypeptide, an inhibitor of a RecA/RAD51 polypeptide, a BRCA-1 polypeptide, a BRCA-2 polypeptide, a RAD50 polypeptide, a DNA break repair polypeptide, a Mgs1 polypeptide, a radiation sensitivity polypeptide, a Pifl1 helicase polypeptide, a Sgs1 helicase polypeptide, a RecQ helicase polypeptide, a Mus51 polypeptide, a Mus52 polypeptide, or a BML polypeptide, and, thereby increases the efficiency of homologous recombination in said eukaryotic cell.

In still further aspects, the composition introduced into the eukaryotic cell comprises a homologous recombination cassette and at least one polypeptide which increases homologous recombination activity in the eukaryotic cell. In specific aspects, the polypeptide which increases homologous recombination activity in the eukaryotic cell is selected from the group consisting of a RecA polypeptide, a Rad54 polypeptide, or a Rad51 polypeptide or any combination thereof.

In another method, the efficiency of homologous recombination of a polynucleotide of interest in a eukaryotic cell is increased. In some aspects the method comprises introducing into the eukaryotic cell a first polypeptide or a first polynucleotide which transiently decreases the level of non-homologous recombination in the eukaryotic cell; and, introducing into the eukaryotic cell a composition comprising a homologous recombination cassette, wherein the polynucleotide of interest is integrated into the target site by a homologous recombination event.

In other methods, the methods generally involve genetically modifying the cell with an expression construct that comprises a nucleotide sequence encoding a siRNA that specifically reduces the level of a gene product that contributes to non-homologous recombination, such that the frequency of non-homologous recombination is reduced, and the efficiency of homologous recombination is increased.

In some embodiments, the methods further involve contacting the exogenous nucleic acid with one or more proteins that contribute to homologous recombination. The one or more proteins provide for homologous recombination between the exogenous nucleic acid and an endogenous nucleic acid within the cell; or between two exogenous nucleic acids. In some embodiments, the exogenous nucleic acid is contacted with the one or more proteins that contribute to homologous recombination, where the contacting occurs inside the cell. In some of these embodiments, a nucleic acid comprising a nucleotide sequence encoding the one or more proteins that contribute to homologous recombination is introduced into the eukaryotic cell. The encoded protein then contacts the exogenous nucleic acid.

In other embodiments, the exogenous nucleic acid is contacted with the one or more proteins that contribute to homologous recombination outside the cell. For example, in some embodiments, the exogenous nucleic acid is contacted with the one or more proteins, forming a nucleoprotein complex, and the nucleoprotein complex is introduced into the eukaryotic cell.

Further provided are kits comprising, for example, a polynucleotide encoding a silencing element, wherein the silencing element when introduced into a eukaryotic cell reduces the level of a gene product that contributes to non-homologous recombination, and, increases the homologous recombination activity in the eukaryotic cell. The kit can further comprise one or more polynucleotides selected from the group consisting of:
a) a polynucleotide comprising a homologous recombination cassette comprising at least a first region having sufficient sequence identity to a corresponding first region of a target site in the eukaryotic cell; or
b) a polynucleotide which when expressed in the eukaryotic cell increases homologous recombination activity in the eukaryotic cell.

In further aspects of the invention, a kit comprising a eukaryotic cell having a decreased level of non-homologous recombination activity is provided, wherein the decreased level of non-homologous recombination activity is transient. The kit can further comprise one or more polynucleotides selected from the group consisting of:
a) a polynucleotide comprising a homologous recombination cassette; or
b) a polynucleotide which when expressed in the eukaryotic cell increases homologous recombination activity in the eukaryotic cell.

In specific embodiments, the homologous recombination cassette in the kit comprises a first region and a second region having sufficient identity to a corresponding second region of the target site.

In further embodiments, the kit comprising a gene product that contributes to non-homologous recombination which can include, for example, a polynucleotide that encodes a Ku70 polypeptide, a Xrcc4 polypeptide, or a Ligase IV polypeptide, a DNA-dependant protein kinase catalytic subunit (DNA-PKcs) polypeptide or an Artemis nuclease polypeptide or any combination thereof.

In further aspects, the silencing element encoded by the polynucleotide in the kit comprises an siRNA silencing element, a protein nucleic acid (PNA) molecule, an miRNA, a double stranded RNA silencing element, a hairpin RNA silencing element, an antisense silencing element or a sense silencing element.

In further aspects, the kit comprises a polynucleotide which when expressed in the eukaryotic cell increases homologous recombination activity is selected from the group consisting of a polynucleotide encoding a RecA polypeptide, a Rad54 polypeptide, or a Rad51 polypeptide or any combination thereof. In further embodiments, the heterologous polynucleotide which when expressed in the eukaryotic cell increases homologous recombination activity comprises a silencing element which reduces the level of a helicase polypeptide, a cell-cycle polypeptide, a Structural Maintenance of Chromosomes (SMC) polypeptide, a topoisomerase polypeptide, an inhibitor of a RecA/RAD51 polypeptide, a BRCA-1 polypeptide, a BRCA-2 polypeptide, a RAD50 polypeptide, a DNA break repair polypeptide, a Mgs1 polypeptide, a radiation sensitivity polypeptide, a Pifl1 helicase polypeptide, a Sgs1 helicase polypeptide, a RecQ helicase polypeptide, a Mus51 polypeptide, a Mus52 polypeptide, or a BML polypeptide, and, thereby increases the efficiency of homologous recombination activity in said eukaryotic cell.

### BRIEF DESCRIPTION OF THE DRAWING(S)

FIG. 1 provides a schematic representation of the experimental design. HCT116 cells were transfected with Ku70 and/or Xrcc4 siRNA at 30-50% confluence. Following siRNA transfection, subsets of cells were subjected to (a) protein analysis (48 or 96 h), (b) cell irradiation (48 h), or (c) re-transfection with lin-pEGFP (48 or 96 h). After additional culture, the cell cycle in irradiated cells (b) and the percentage of GFP-expressing cells (c) were measured by flow cytometry at 72 h and at 120 or 192 h, respectively. Colony formation was determined in irradiated cells at 240 h (10 days) post-siRNA transfection.

FIG. 2 demonstrates the effects of RNAi depletion of Ku70 and Xrcc4 as measured by protein densitometry. GAPDH was used as endogenous control. (a) Relative abundance of Ku70 and Ku80 proteins in HCT116 cells following siRNA transfections for Ku70 and/or Xrcc4. Control: non-treated cells; Stealth: non-specific siRNA; Ku70: siRNA specific to Ku70; XR: siRNA specific to Xrcc4; XR-Ku: siRNAs specific to each Xrcc4 and Ku70, at half the doses used for individual transfections (co-transfection). (b) Relative abundance of Ku70 and Xrcc4 proteins in HCT116 cells following siRNA transfections for Ku70 and/or Xrcc4. Lanes: 1—non-treated cells; 2—non-specific siRNA; 3 to 5—siRNA specific to Ku70; 6 to 8—siRNA specific to Xrcc4; 9—siRNAs specific to each Xrcc4 and Ku70, at half the doses used for individual transfections (co-transfection). (c to e) Relative abundance of NHEJ proteins at 48 and 96 h after siRNA-treated samples for Ku70 (c), Ku80 (d) and Xrcc4 (d). Ku70 RNAi resulted in the concomitant decrease of Ku70 and Ku80 but not Xrcc4 protein (48 h). Depletion of Xrcc4 by RNAi resulted in the decrease of both Ku70 and Ku80 proteins (48 h). Rad51 and Chk1 proteins levels did not change in siRNA-treated samples (data not shown).

FIG. 3 provides the ell cycle analyses of siRNA-treated HCT116 cells before and 24 hours after γ irradiation. HCT116 cells were transfected with Ku70, Xrcc4 and nonspecific siRNA or mock transfected with Lipofectamine 2000 alone (Sham). Cells were treated with 8 Gy of γ radiation 48 h post-siRNA transfection. Irradiated and non-irradiated cells siRNA-treated cells were subjected to cell cycle analysis by flow cytometry. Cell cycle analyses of irradiated cells revealed significantly decrease and increase in the proportion of cells at the S and G2/M phases, respectively. Ku70 and Xrcc4 depleted cells responded to DNA damage by activating a major cell cycle checkpoint (G2/M) due to inability to efficiently repair DSBs, suggesting a stronger G2/M checkpoint in siRNA-treated cells. Among the non-irradiated population, the transfection procedure and not the siRNA treatment appeared to have affected the distribution of the cells in the different stages of the cell cycle. Depletion of Ku70 and Xrcc4 caused increased sensitivity to γ radiation. Results were obtained from triplicates in 4 independent experiments and represent the means and standard deviations.

FIG. 4 provides the survival analysis after γ radiation. HCT116 cells were transfected with Ku70, Xrcc4 and nonspecific siRNA or mock transfected with Lipofectamine 2000 alone (Sham). Cells were treated with 8 Gy of γ radiation 48 h post-siRNA transfection. Cells were plated in triplicate for colony formation assay. The means and standard deviations for 4 independent experiments are presented. The increase in sensitivity to γ radiation demonstrates a reduced DNA repair capacity in siRNA-treated-radiated cells due to depletion of Ku70 and Xrcc4 proteins.

FIG. 5 shows the relative proportion of GFP-expressing HCT116 cells following lin-pEGFP transfections 48 or 96 h after Ku70 and/or Xrcc4 siRNA transfection. Results are from flow cytometry performed 72 h after lin-pEGFP transfection (120 or 192 h post-siRNA transfection). Control: non-treated cells; Sham: 0 nM siRNA; Stealth: non-specific siRNA; Ku70: siRNA specific to Ku70; Xrcc4: siRNA specific to Xrcc4; Ku70-Xrcc4: siRNAs specific to each Ku70 and Xrcc4 (co-transfection). The DNA integration assay estimates the ratio of lin-pEGFP integration by levels of fluorescence after 72 hours of DNA transfection, which were performed in duplicates in at least 4 replicates for each siRNA treatment and each time point (48 or 96 h). The Ku70 and Xrcc4 down regulation by RNAi in HCT116 cells negatively affected lin-pEGFP integration at 48 h post-siRNA transfection.

FIG. 6 provides a non-limiting Experimental strategy. Linearized HPRT/hyg targeting construct excised from the plasmid, will first be treated with T7 gene 6 exonuclease to produce single-stranded 3′ overhangs of up to 500 bp. Then, the modified vector will be either left naked (non-coated) or coated with RecA, hRad51 or hRad51+hRad54 recombination enzymes, to be used for subsequent cell transfection by lipofection. For the selection of stably transformed cells, cultures grouped by coating treatment will be treated 24 h after transfection with either 100 μg/mL hygromycin or hygromycin and 15 μg/mL 6-TG (selection of randomly and homologously recombined cells or strictly homologously recombined cells, respectively). Untreated (wild type) cells will be included in the selection as controls. After one week, the number of colonies will be counted. Selected stable colonies will be confirmed by PCR using specific primers to amplify the hyg gene. HR will be confirmed by Southern blot analyses in colonies resistant to both hygromycin and 6-TG.

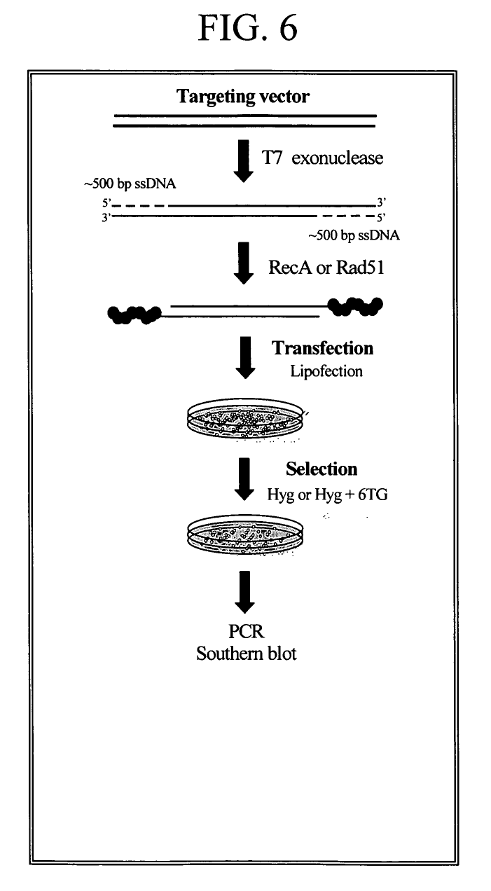

FIG. 7 provides a gene targeting approach at the human HPRT locus. a) Strategy for targeting the HPRT locus; HPRT is an X-linked, single copy gene in male cells; its inactivation leads to 6-thioguanine (6-TG) resistance. A replacement type targeted vector was used (pHPRT); this vector was produced by the insertion of the hygromycin B phosphotransferase expression cassette (hygro) within HPRT exon II. HCT116 cells were transfected with a linearized pHPRT vector 48 h after siRNA transfection for Ku70 or Xrcc4. b) PCR-based gene targeting assay. Gene targeting events were detected by PCR with a pair of oligonucleotides designed to amplify a 2.5-kb fragment diagnostic of gene targeting at the exon II (representing the hygromycin cassette). Gene targeting positive colonies presented only the 2.5 kb band. If the construct integrated by illegitimate recombination as shown in the hygromycin selected colonies the PCR reaction will produce the 2.5 kb band from the construct plus a 200 bp from the HPRT endogenous allele. Clones were scored as targeted at the HPRT locus if only the 2.5 kb band was detected, and counted as random events if both HPRT-derived, 2.5-kb and 200 bp bands were observed. The locations of the PCR primer pairs, used to detect HR are shown as P1 and P2. The figure illustrates an example of this assay applied to colonies transformed with the pHPRT targeting vector and control cells. Lanes: 1) 100 bp DNA ladder; 2, 5 and 6)Hygromycin only selected colony; 3 and 4) 6TG selected colonies; 7) HCT116 non-transfected; 8)pHPRT vector; 9) 1 kb DNA ladder.

### DETAILED DESCRIPTION OF THE INVENTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

I. Overview

Methods and compositions for increasing or decreasing the efficiency of homologous recombination activity in a eukaryotic cell are provided. Such methods and compositions can be employed to allow for the targeted integration of a polynucleotide of interest at a predetermined locus or a random locus in a host cell. By modulating the level of one or more (1, 2, 3, 4, 5, 6, 7, 8 or more) gene products which influence non-homologous recombination, the present invention demonstrates that a reduction in random DNA integration in a cell provides an environment which promotes homologous recombination events. Accordingly, various methods and compositions are provided which decrease or increase the level of non-homologous recombination in a cell. In specific aspects, the efficiency of targeted homologous recombination events is increased.

As used herein, “homologous recombination” or “HR” or “legitimate recombination” comprises the exchange of DNA-sequences between two DNA molecules which share a sufficient level of sequence identity. Accordingly, homologous recombination can be exploited to allow for the targeted insertion of a polynucleotide of interest into a predetermined locus in a cell. As used herein, “non-homologous recombination” or “illegitimate recombination” comprises the exchange of DNA sequences between two DNA molecules which share little to no sequence homology. As the present invention demonstrates that decreasing non-homologous recombination events results in an increase in the number of homologous recombination events, methods are provided which increase the homologous recombination activity in a cell. An increase in homologous recombination activity comprises any statistically significant increase the efficiency of homologous recombination activity in a cell when compared to an appropriate control. Such increases can include, for example, at least a 5%, 10%, 20%, 30%, 40%, 50%, 60%, 70%, 80%, 90%, 100%, 150%, 200% or greater increase in the efficiency of a homologous recombination event of a polynucleotide of interest. Such increases can also include, for example, at least about a 3%-15%, 10%-25%, 20% to 35%, 30% to 45%, 40%-55%, 50%-65%, 60%-75%, 70%-85%, 80%-95%, 90%-105%, 100%-115%, 105%-120%, 115%-130%, 125%-150%, 140%-160%, 155%-500% or greater increase in the efficiency of a homologous recombination event of a polynucleotide of interest. Methods to assay for the efficiency of homologous recombination activity are known. See, for example, Wright et al. (2005) Plant J. 44:693-705; Bell et al. (2003) J. Biol. Chem. 278:45182-45188; Snouwaert et al. (1999) Oncogene 18:7900-7; Roth et al. (1985) Proc. Natl. Acad. Sci. USA 82:3355-9; and U.S. Pat. No. 6,562,624, each of which is herein incorporated by reference. The rate or efficiency of homologous recombination can be expresses as an increase in the number of HR events per unit DNA. Homologous recombination activity can also be measured by assaying the ratio of homologous recombination events to non-homologous recombination events in a cell. In specific aspects, the ratio of homologous recombination events to non-homologous recombination events is increased by about 1, 2, 3, 4, 5, 6, 7, 8, 9, 10, 20, 30, 40, 50, 60, 70, 80, 90, 100, 300, 600, 900, 1000 fold or greater when compared to an appropriate control cell. In other aspects, the ratio of homologous recombination events to non-homologous recombination events is increased by about 1 to 5 fold, about 5 to 10 fold, about 10 to 20 fold, about 20 to 30 fold, about 30 to 40 fold, about 40 fold to 60 fold, about 60 fold to 80 fold, about 80 fold to about 100 fold, about 100 to 200 fold, about 200 fold to 300 fold, about 300 to 400 fold, about 400 to about 500 fold, about 500 to about 500 fold, about 500 fold to about 700 fold, about 700 fold to 800 fold, about 800 fold to about 1000 fold or greater when compared to an appropriate control. Methods to determine the ratio of non-homologous to homologous recombination events can be found, for example, in WO2005/108586, herein incorporated by reference. Accordingly, in specific embodiments methods and compositions are provided which modulate (increase or decrease) the ratio of homologous to non-homologous recombination events.

II. Decreasing the Level of Non-Homologous Recombination Activity

Methods and compositions of the present invention include those which decrease non-homologous recombination activity in cell, and thereby increase homologous recombination activity. As outlined in further detail elsewhere herein, in specific embodiments, the decrease in non-homologous recombination activity is transient. As used herein, a decrease in the level of non-homologous recombination activity comprises any statistically significant decrease in the level of non-homologous recombination activity in a cell when compared to an appropriate control. Such decreases can include, for example, at least a 5%, 10%, 20%, 30%, 40%, 50%, 60%, 70%, 80%, 90%, or 100% decrease in the level of non-homologous recombination activity. Methods to assay for this activity are known and include, for example, an increased sensitization to radiation or a decrease in the random stable integration of a DNA construct/transgene into the cell. Additionally, assays have been described in the art which assess repair of double-stranded breaks. These methods use a plasmid (e.g., containing a reporter gene) linearized by restriction enzyme digestion as a model of the double stranded break. This substrate may be transfected into cells, as described in Collis et al. ((2002) Nucleic Acids Res 30:e1). Joining can also be assessed in vitro using cell extracts. An additional NHEJ assay has been described by Baumann and West ((1998) Proc. Natl. Acad. Sci. USA 95: 14066-14070) in which a radiolabeled linearized plasmid is used as substrate. Linear dimers and trimers produced following joining can be separated on agarose gels prior to quantification. A modification of this assay that requires smaller amounts of starting material, as well as utilizes SYBR Green dye rather than radiochemical is described in Diggle et al. (2003) Nucleic Acids Res 31(15):e83. Each of these references is herein incorporated by reference in their entirety.

A compound that inhibits non-homologous recombination activity can comprise a polypeptide, nucleic acid, antibody, small molecule, or other compound which when contacted or introduced into a cell decreases the level of non-homologous recombination activity, either directly or indirectly, in that cell. Accordingly, various methods which employ one or more of such compounds can be used to decrease the level of non-homologous recombination activity.

Non-homologous recombination requires several factors that sequentially recognize and bind the broken ends, catalyze the synapses and then process and reseal the break (Lees-Miller et al. (2003) Biochimie 11:1161-1173, Lieber et al. (2003) Nat. Rev. Mol. Cell. Biol. 4:712-720, and Paques et al. (1990) Microbiol. Mol. Biol. Rev. 63:349-404). Various factors involved in this pathway have been characterized. In one embodiment, the non-homologous recombination inhibiting compound which is contacted with or introduced into the cell increases the level of a polypeptide whose activity inhibits non-homologous recombination. Polypeptides which decrease non-homologous recombination activity when their level is increased are known. Various methods for over expressing a polynucleotide or polypeptide of interest are disclosed elsewhere herein.

In other embodiments, the non-homologous recombination inhibiting compound which is contacted or introduced into the cell decreases the level of a polypeptide whose activity inhibits non-homologous recombination. Polypeptides which decrease non-homologous recombination activity when their level is decreased are known. Such polypeptides include, but are not limited to, proteins of the non-homologous end joining (NHEJ) pathway. Non-limiting members of this pathway are discussed below as are various methods for decreasing the level of the various gene products. While any method can be used to decrease the level of the targeted gene product, in one embodiment, silencing elements directed to members of the NHEJ pathway are employed. It is recognized that the level and/or activity of a gene product from at least 1, 2, 3, 4, 5, 6 or more members of the NHEJ pathway can be decreased in the methods and compositions. As used herein, a “non-homologous recombination silencing element” refers to a silencing element that is capable of reducing or eliminating the level (i.e., inhibiting the expression) of a polypeptide at the level of transcription and/or translation that promotes non-homologous recombination.

At least six different molecules are required for NHEJ: Ku70, Ku86, DNA-PKcs, XRCC4, DNA Ligase IV, and Artemis. Three of these molecules comprise the DNA-dependent protein kinase (DNA-PK) complex, which is a serine/threonine protein kinase that contains two subunits, DNA-PKcs and Ku. The DNA-PK catalytic subunit (DNA-PKcs) possesses weak DNA-binding activity as well as protein kinase activity (Hartley et al (1995) Cell 82:849-856 and Yaneva et al. (1997) EMBO Journal 16:1598-5112). The regulatory subunit, Ku heterodimer, directs DNA-PKcs to DNA ends and stabilizes its DNA binding so that it is efficiently activated (Gottlieb and Jackson (1993) Cell 72:131-142; Suwa et al. (1994) Proc. Natl. Acad. Sci. USA 91:6904-6908). This structure-specific DNA binding protein requires a free DNA end for binding to occur (Mimori and Hardin (1986) J Biol. Chem. 261(22):10375-9; and, Yoo et al. (1999) Nucleic Acids Res. 27(24):4679-86). Ku binds to DNA ends in a sequence-independent manner and then translocates to internal sites (Dynan and Yoo (1998) Nucleic Acids Res 26:1551-1559). XRCC4 interacts with and catalytically stimulates the activity of DNA Ligase IV (also referred to herein as “Ligase IV”). The XRCC4-DNA Ligase IV complex carries out the final step of joining DNA ends in NHEJ. The sixth factor, Artemis, plays an important role in DNA end-processing during NHEJ and specifically during V(D)J recombination (reviewed in Meek et al. (2004) Immunol Rev 200:132-141).

The Ku heterodimer consists of the Ku70 and Ku86 (also referred to as “Ku80”) polypeptides. Various Ku70 polypeptides (native or biologically active variants or fragments thereof) are known to be involved in non-homologous recombination and can be targeted for suppression using the Ku70 silencing element. Ninomiya et al. ((2003) Proc Natl Acad Sci USA 17; 101(33):12248-53) recently demonstrated that mutation of either of two Neurospora genes required for nonhomologous endjoining DNA repair (mus-51 and mus-52) results in a dramatic increase in the frequency of homologous recombination. Mus-51 and mus-52 are homologs of Ku70 and Ku80 polypeptides.

The crystallization of the Ku heterodimer revealed that the protein forms a ring tethering the DNA ends (Walker et al. (2001) Nature 412(6847):607-14). The Ku70 polypeptide and the gene encoding the polypeptide are known. See, Accession No. NM_{—}001469 of the National Center for Biotechnology Information (NCBI) (SEQ ID NO:1 and 2), which is herein incorporated herein by reference in its entirety. Active variants and fragment of Ku70 have been identified. See, for example, Takiguchi et al. (1996) Genomics 35(1):129-35 which provides the sequence of mouse Ku70 gene and Jin et al. (1997) EMBO J. 16:6874-6885 which provides an extensive mutational analysis of the human Ku70 gene. Additionally, Ku70 homologs in other species have been identified, including mouse (NCBI Accession No. NM_{—}010247, SEQ ID NO:3 and 4), pig (TIGR porcine Gene Index Accession No. TC200078, SEQ ID NO:5 and 6), rat (NCBI Accession No. NP_{—}620780), hamster (NCBI Accession No. AAB46854), and chicken (NCBI Accession No. NP_{—}990258). Each of these references is herein incorporated by reference.

In some embodiments, the “Ku70 silencing element” can be used to target suppression of NHEJ. The “Ku70 silencing element” refers to a silencing element that is capable of reducing or eliminating the level (i.e., inhibiting the expression) of a Ku70 polypeptide at the level of transcription and/or translation. Various Ku70 polypeptides (native or biologically active variants or fragments thereof) are known and can be targeted for suppression using the Ku70 silencing element. A silencing element of the invention can be designed to reduce or eliminate expression of a native Ku70 sequence, or alternatively, the silencing element can be designed to reduce or eliminate expression of a sequence encoding a biologically active variant or fragment of Ku70.

Ku86 heterodimerizes with Ku70 to form the Ku heterodimer. Ku86 participates in NHEJ by supporting alignment of the broken ends of the DNA and thus maintaining integrity of the fragmented chromatin (Dmitrieva, et al. (2005) Proc. Natl. Acad. Sci. USA 102(30):10730-10735). The Ku86 polypeptide and the gene encoding the polypeptide are known. See, NCBI Accession No. NP_{—}066964. Ku86 has been identified in other species, including for example, mouse (NCBI Accession No. AAH51660) and hamster (NCBI Accession No. AAC52664). Each of these references is herein incorporated by reference.

In some embodiments, the “Ku86 silencing element” can be used to target suppression of NHEJ. A “Ku86 silencing element” refers to a silencing element that is capable of reducing or eliminating the level (i.e., inhibiting the expression) of a Ku86 polypeptide at the level of transcription and/or translation. Various Ku86 polypeptides (native or biologically active variants or fragments thereof) are known and can be targeted for suppression using the Ku86 silencing element. The silencing element of the invention can be designed to reduce or eliminate expression of a native Ku86 sequence. Alternatively, the silencing element can be designed to reduce or eliminate expression of a sequence encoding a biologically active variant or fragment of Ku86.

The Xrcc4 polypeptide and the gene encoding the polypeptide are known. See, NCBI Accession No. NM_{—}003401 (SEQ ID NO:7 and 8). Active variants and fragments of Xrcc4 have been identified. See, for example, NCBI Accession No. NP_{—}003392 and AAP36649. Additionally, Xrcc4 has been identified in other species, including mouse (NCBI Accession No. AAH25538 and NCBI Accession No. NM_{—}028012, SEQ ID NO:9 and 10) and pig (TIGR porcine Gene Index Acc. No. TC204995, SEQ ID NO:11 and 12). Each of these references is herein incorporated by reference.

In some embodiments, the “Xrcc4 silencing element” can be used to target suppression of NHEJ. An “Xrcc4 silencing element” refers to a silencing element that is capable of reducing or eliminating the level (i.e., inhibiting the expression) of an Xrcc4 polypeptide at the level of transcription and/or translation. Various Xrcc4 polypeptides (native or biologically active variants or fragments thereof) can be targeted for suppression using the Xrcc4 silencing element. The silencing element of the invention can be designed to reduce or eliminate expression of a native Xrcc4 sequence. Alternatively, the silencing element can be designed to reduce or eliminate expression of a sequence encoding a biologically active variant or fragment of Xrcc4.

The DNA Ligase IV polypeptide and the gene encoding the polypeptide are known. See, NCBI Accession No. NP_{—}996820. Active variants and fragment of Ligase IV have been identified. See, for example, Girard et al. (2004) Hum Mol Genet. 13(20):2369-76; Roddam et al. (2002) J. Med. Genet. 39(12):900-5; and, Kuschel et al. (2002) Hum Mol. Genet. 11(12):1399-407, which provide extensive mutational analyses of the human Ligase IV gene. Additionally, Ligase IV has been identified in other species, including rat (NCBI Accession No. NP_{—}620780), mouse (NCBI Accession No. NP_{—}795927), hamster (NCBI Accession No. AAB46854), pig (TIGR porcine Gene Index Accession No. TC209183), and chicken (NCBI Accession No. NP_{—}990258). Each of these references is herein incorporated by reference.

In some embodiments, the “Ligase IV silencing element” can be used to target suppression of NHEJ. A “Ligase IV silencing element” refers to a silencing element that is capable of reducing or eliminating the level (i.e., inhibiting the expression) of a Ligase IV polypeptide at the level of transcription and/or translation. Various Ligase IV polypeptides (native or biologically active variants or fragments thereof) are known and can be targeted for suppression using the Ligase IV silencing element. The silencing element of the invention can be designed to reduce or eliminate expression of a native Ligase IV sequence. Alternatively, the silencing element can be designed to reduce or eliminate expression of a sequence encoding a biologically active variant or fragment of Ligase IV.

The DNA-PKcs polypeptide and the gene encoding the polypeptide are known. See, NCBI Accession No. NP_{—}008835. Active variants and fragment of DNA-PKcs have been identified. See, for example, NCBI Accession No. AAC50210. Mutational analyses of the DNA-PKcs gene can be found in, for example, Convery et al. (2005) PNAS 102(5):1345-1350. Additionally, DNA-PKcs has been identified in other species, including mouse (NCBI Accession No. BAA28873) and pig (TIGR porcine Gene Index Accession No. TC208041). Each of these references is herein incorporated by reference.

In some embodiments, the “DNA-dependent protein kinase catalytic subunit silencing element” can be used to target suppression of NHEJ. A “DNA-dependent protein kinase catalytic subunit silencing element” or a “DNA-PKcs silencing element” refers to a silencing element that is capable of reducing or eliminating the level (i.e., inhibiting the expression) of a DNA-PKcs polypeptide at the level of transcription and/or translation. Various DNA-PKcs polypeptides (native or biologically active variants or fragments thereof) are known and can be targeted for suppression using the DNA-PKcs silencing element. The silencing element of the invention can be designed to reduce or eliminate expression of a native DNA-PKcs sequence. Alternatively, the silencing element can be designed to reduce or eliminate expression of a sequence encoding a biologically active variant or fragment of DNA-PKcs.

The Artemis (or “Artemis nuclease”) polypeptide and the gene encoding the polypeptide are known. See, NCBI Accession No. CAC37570. Active variants and fragment of Artemis have been identified. See, for example, NCBI Accession Nos. NP_{—}001029030, NP_{—}001029029, NP_{—}001029027, and NP_{—}071932. Additionally, Artemis has been identified in other species, including chicken (NCBI Accession No. AAR27406) and mouse (NCBI Accession No. Q8K4J0). Each of these references is herein incorporated by reference.

In some embodiments, the “Artemis silencing element” (also referred to herein as “Artemis nuclease silencing element”) can be used to target suppression of NHEJ. An “Artemis nuclease silencing element” refers to a silencing element that is capable of reducing or eliminating the level (i.e., inhibiting the expression) of an Artemis nuclease polypeptide at the level of transcription and/or translation. Various Artemis nuclease polypeptides (native or biologically active variants or fragments thereof) are known and can be targeted for suppression using the Artemis nuclease silencing element. The silencing element of the invention can be designed to reduce or eliminate expression of a native Artemis nuclease sequence. Alternatively, the silencing element can be designed to reduce or eliminate expression of a sequence encoding a biologically active variant or fragment of Artemis nuclease.

The activity and/or the level of at least 1, 2, 3, 4, 5 or each of a Ku70 polypeptide, a Ku86 polypeptide, a DNA-PKcs polypeptide, a XRCC4 polypeptide, a DNA Ligase IV polypeptide, or an Artemis polypeptide can be decreased in the methods, compositions and kits disclosed herein.

III. Modulating the Level of a Gene Product that Contribute to Homologous Recombination

The efficiency of homologous recombination can be increased by modulating the level (increasing or decreasing) of gene products involved in or associated with DNA repair other than (or in addition to) the NHEJ-associated targets described above. Homologous recombination is a major pathway for the elimination of DNA double-strand breaks induced by high-energy radiation and chemicals, or that arise due to endogenous damage and stalled DNA replication forks. Therefore, a variety of gene products can be modulated (increased or decreased) such that homologous recombination is favored. Such modulation can be performed so that is occurs constitutively or transiently. Methods for transient modulation of gene activity are discussed elsewhere herein. The targets described below are intended to be a representative rather than exhaustive list of gene products encompassed by the present invention. A review of many of the genes involved in DNA repair in humans can be found in Wood et al. (2005) Mutation Research 577(1-2):275-83 or on the world-wide web at www.cgal.icnet.uk/DNA_Repair_Genes. In specific embodiments, the level and/or activity of a polypeptide or polynucleotide which modulates double stranded breakage (either targeted or non-targeted) can be employed. In still other embodiments, the polypeptide or polynucleotide that increases homologous recombination activity in the cell does not increase targeted and/or non-targeted double-stranded breakage at the target site.

In the methods and compositions, the efficiency of homologous recombination can be increased by modulating the level (increasing or decreasing) at least 1, 2, 3, 4, 5, 6, 7, or more gene products involved in or associated with DNA repair other than (or in addition to) the NHEJ-associated targets described above. For example, in specific embodiments, the level of the BLM polypeptide or an active variant or a fragment thereof is decreased and the level of the Ku70 polypeptide or an active variant or fragment thereof is decreased. In still other embodiments, the level of a SMC5 polypeptide or an active variant or a fragment thereof is decreased and the level of the Ku70 polypeptide or an active variant or fragment thereof is decreased.

A. Rad 50

In one embodiment, the level of a Rad50 polypeptide is modulated (increased or decreased) using any of the methods to decrease the level of a gene product described elsewhere herein. Rad50 is a polypeptide involved in DNA double-strand break repair. This protein forms a complex with MRE11 and NBS1. The protein complex binds to DNA and displays numerous enzymatic activities that are required for non-homologous joining of DNA ends. Recent studies of the architecture of the human and Pyrococcus furiosis MRE11-RAD50 complexes revealed that they have a structural role in bridging DNA ends, and possibly sister chromatids, through the coiled-coil regions of RAD50 (Hopfner et al. (2002) Nature 418(6897):562-566). The Rad50 polypeptide and the gene encoding the polypeptide are known. Various Rad50 polypeptides (native or biologically active variants or fragments thereof) are known. See, NCBI Accession No. NM_{—}005732. Each of these references is herein incorporated by reference.

B. Bloom Syndrome Protein

In another embodiment, the level of Bloom syndrome protein (BLM) is decreased using any of the methods to decrease the level of a gene product described elsewhere herein. BLM encodes a homolog of the Escherichia coli DNA helicase (Ellis et al. (1995) Cell 83: 655-666; Harmon et al. (1998) Genes Dev. 12: 1134-1144). This DNA helicase unwinds double-stranded DNA molecules, a process required for various aspects of DNA metabolism, including transcription, DNA repair, and replication (Hickson (2003) Nat. Rev. Cancer, 3:169-178). BLM knockout cells showed an increased tendency of sister chromatids to exchange DNA strands and were substantially more likely to undergo homologous recombination at chromosomal loci than parental cells (Traverso et al. (2003) Cancer Research 63:8578-8581). The BLM polypeptide (native or biologically active variants or fragments thereof) are known, as well as, the genes encoding the polypeptides are known. See, NCBI Accession No. NM_{—}000057. Each of these references is herein incorporated by reference.

C. Structural Maintenance of Chromosomes (SMC) Family

Further embodiments of the present invention include modulation (increase or decrease) of the Structural Maintenance of Chromosomes (SMC) family of proteins. The SMC family of proteins is essential for successful chromosome transmission during replication and segregation of the genome in all organisms. The SMC superfamily proteins (PFAM Accession No. PF02463) have ATP-binding domains at the N- and C-termini, and two extended coiled-coil domains separated by a hinge in the middle. The six eukaryotic core SMCs (SMC1-SMC6) form functional complexes with other proteins. SMC1 and SMC3 are part of the cohesion complex, which contains two other proteins (sister-chromatid cohesion proteins Scc1 and Scc3) and is required for sister-chromatid cohesion during mitosis. The SMC1-SMC3 dimer also forms a recombination complex (RC-1) with DNA polymerase ε and ligase III (Jessberger et al. (1996) EMBO J. 15:4061-4068). Potts et al. (EMBO J 1-12; epub Jun. 29, 2006) describe a model in which the SMC5/6 complex localizes to sites of DNA damage and promotes the recruitment of the cohesion complex to the double-stranded break. This SMC5/6-facilitated recruitment of cohesion to the double-stranded break holds the sister chromatids in close proximity to permit RAD51-dependent strand invasion and exchange using the sister chromatid as the repair template. Inhibition of the SMC5/6 or cohesion complexes reduces sister chromatid homologous recombination, thereby shifting the double-stranded break repair pathway to NHEJ or homologous recombination with a heterologous template. A decrease in cohesion between sister chromatids can facilitate pairing and recombination of heterologous DNA.

The SMC polypeptides (native or biologically active variants and fragments thereof) and the genes encoding the polypeptides (or homologs thereof) have been identified in various organisms. See NCBI Accession Nos. NM_{—}006306 (SMC1A); NM_{—}148674 (SMC1B); BC032705 (SMC2); NM_{—}005445 (SMC3); NM_{—}133786 (SMC4); NM_{—}015110 (SMC5); and NM_{—}025695 (SMC6). Additional pairing proteins (i.e., proteins involved in stabilizing and bring DNA strands together for repair or recombination could also increase homologous recombination activity). In one embodiment, increasing the level of expression of inhibitors of pairing proteins could be employed.

In addition, as with the Structural Maintenance of Chromosomes (SMC family) proteins discussed above, the level and/or activity of RNA or other factors involved in chromatin structure, like CTCF (Ling et al. (2006) Science 312: 269, herein incorporated by reference) could be modulated (increased or decreased) to increase further increase homologous recombination. Inhibition of such a polypeptides can make it easier for a foreign piece of DNA to find its homologous site in the genome, or prevent homologous sequences from coming together. In the later case overexpression would be preferred.

D. Topoisomerases

DNA topoisomerases (native and biologically active variants thereof) are a class of enzymes involved in the regulation of DNA supercoiling. Type I topoisomerases change the degree of supercoiling of DNA by causing single-strand breaks and re-ligation, whereas type II topoisomerases (such as bacterial gyrase) cause double-strand breaks. DNA topoisomerases solve the topological problems associated with DNA replication, transcription, recombination, and chromatin remodeling by introducing temporary single- or double-strand breaks in the DNA. In addition, these enzymes maintain the steady-state level of DNA supercoiling both to facilitate protein interactions with the DNA and to prevent excessive supercoiling that is deleterious. As strands are unwound by the RNA polymerase, positive superhelical tension is created in front and negative superhelical tension is created behind the polymerase. In both locations, topoisomerases relieve the superhelical tension.

In yeast, the type IA topoisomerase, Top3p, may work coordinately with Sgs1p in removing Holliday junction intermediates from a crossover-producing recombination pathway (Tsai et al. (2006) J Biol. Chem. 281(19):13717-23). Sgs1 is a RecQ family DNA helicase required for genome stability in Saccharomyces cerevisiae whose human homologs BLM, WRN, and RECQL4 are mutated in Bloom's, Werner, and Rothmund Thomson syndromes, respectively (Schmidt et al. (2006) Mol. Cell. Biol. 14:5406-5420). In top3 mutants, the DNA helicase Pif1 has been shown to interact with Sgs1 to suppress the hyper-recombination effects due to the loss of top3 expression in a recombination-dependent manner (Wagner et al. (2006) Genetics Jul 2 epub).

In one embodiment of the present invention, the efficiency of homologous recombination activity is increased by decreasing or increasing the level of topoisomerase enzymes in a manner that increases negative superhelical tension (e.g., by decreasing the enzyme that resolves the negative superhelical tension) to allow access of heterologous DNA to the genome for recombination. Various topoisomerase polypeptides (native and biologically active variants and fragments thereof) and the genes encoding the polypeptides are known. See, NCBI Accession Nos. NP_{—}003277 (topoisomerase I); NP_{—}001058 (topoisomerase IIA); NP_{—}001059.2 (topoisomerase IIB); NP_{—}004609.1 (topoisomerase IIIA); and, NP_{—}003926.1 (topoisomerase IIIB). The Saccharomyces cerevisiae gene encoding Pif1 can be found in NCBI Accession No. CAA86260. The Saccharomyces cerevisiae gene encoding Sgs1 can be found in NCBI Accession No. AAB60289. Each of these references is herein incorporated by reference.

E. Modulation of RAD6/RAD52 Pathways

Central to the process of error-free homologous recombination are the RAD52 group genes (RAD50, RAD51, RAD52, RAD54, RDH54/TID1, RAD55, RAD57, RAD59, MRE11, and XRS2). In contrast, the RAD6 DNA damage tolerance pathway facilitates double stranded DNA break repair via error-prone translesion synthesis.

Srs2 helicase was recently shown to block the RAD52-dependent homologous recombination by actively disrupting the Rad51-nucleoprotein filament (Hishida et al. (2006) Mol Cell Biol 26(14):5509-5517). Sumoylated PCNA facilitates this disruption by recruiting the Srs2 helicase to replication forks and helping it to prevent the RAD52-dependent recombination during S phase. The DNA-dependent ATPase Mgs1 physically associates with PCNA and acts to prevent the RAD6 DNA damage tolerance pathway in the absence of exogenous DNA damage. As such, a further aspect of this invention comprises disruption of the RAD6 pathway (e.g., by increasing the level of any factor that favors the RAD52 pathway or by decreasing the level of any factor that favors the RAD6 pathway). In one embodiment, the level of Mgs1 is increased. In another embodiment, the level of Srs2 helicase is decreased.

The nucleotide and amino acid sequences of the RAD52 family (native or biologically active variants or fragments thereof) can be found in NCBI Accession Nos. NM_{—}005732 (RAD50); NM_{—}002875 (RAD51, SEQ ID NO:13 and 14); NP_{—}035364 (Mus muscularis RAD51, SEQ ID NO:15 and 16); NM_{—}002879 (RAD52); NM_{—}003579 (RAD54L, SEQ ID NO:19 and 20); CAA66380 (Mus muscularis RAD54, SEQ ID NO:21 and 22); NM_{—}012415 (RAD51B); CAA88534 (Saccharomyces cerevisiae RDH54/TID1); BAA01284 (Saccharomyces cerevisiae RAD55); NP_{—}005423 (RAD57); CAA98622 (Saccharomyces cerevisiae RAD59); NM_{—}005590 (MRE11); and CA56687 (Saccharomyces cerevisiae XRS2), and TIGR porcine Gene Index Accession Nos. TC209977 (Sus scrofa RAD51, SEQ ID NO:17 and 18) and TC220911 (Sus scrofa RAD54, SEQ ID NO:23). Mgs1 from Saccharomyces cerevisiae can be found in NCBI Accession No. L22856; Srs2 helicase from Saccharomyces cerevisiae can be found in NCBI Accession No. P40151. Each of these references is herein incorporated by reference.

F. RecA/Rad51

In another embodiment of the present invention, the level of a protein in the RecA/Rad51 family of proteins is modulated (increased or decreased) using any of the methods to increase the level of a gene product described elsewhere herein. The RecA/Rad51 family of genes (including RAD51B, RAD51C, RAD51D, XRCC2 and XRCC3) participates in a common DNA damage response pathway associated with the activation of homologous recombination and double-strand break repair. These proteins complex with phosphorylated BRCA1, BRCA2 and RAD52 (native or biologically active variants thereof) for DNA repair of double strand breaks through homologous recombination. In one embodiment, the level and/or activity of RAD51 (native or biologically active variant or fragment thereof) is increased. In another embodiment, the level and/or activity of the RAD51 polypeptide is increased and the level and/or activity of a polypeptide in the NHEJ pathway is decreased (i.e., the level and/or the activity the Ku70 polypeptide, the Ku86 polypeptide, the Xrcc4 polypeptide, the DNA ligase IV polypeptide, the DNA-PKcs polypeptides, or the Artemis polypeptide.)

The RecA/Rad51 family of polypeptides (native and biologically active variants and fragments thereof) and the genes encoding the polypeptides are known. See, NCBI Accession Nos. NM_{—}002877 (RAD51B); NM_{—}002876 (RAD51C); NM_{—}002878 (RAD51D); NM_{—}005431 (XRCC2); NM_{—}005432 (XRCC3); NM_{—}007295 (BRCA1); and, NM_{—}000059 (BRCA2), each of which is herein incorporated by reference. It is recognized that additional DNA break polypeptides can be modulated to increase the homologous recombination activity in the cell. Further, the level of gene products that influence radiation sensitivity can also be modulated to increase homologous recombination activity in a cell.

BRCA 1 and 2 interact with the Rad50/MrebII/Nbs I complex to repair DNA both by homologous recombination and by non-homologous recombination with NHEJ polypeptides such as the Ku, Xrcc4 ligase etc proteins. Inhibition of any of these genes can result in a decrease in non-homologous recombination. See, for example, Zhong et al. (2002) Cancer Research 62:3966-3970, herein incorporated by reference. In other embodiments, the homologous recombination frequency could be increased by increasing the expression of these proteins due to their involvement in homologous recombination. As an example, regulation of BRCA-1 has been extensively studied. See, for example, Farmer et al. (2005) Nature 434:917-21, herein incorporated by reference. Nonhomologous recombination and homologous recombination use many of the same factors/proteins and their regulation in coordinated. Therefore, targeting genes for inhibition could result in the overexpression of other proteins. For example, by decreasing Ku70, an increase in the frequencies of homologous recombination is seen. See also, Spurgers et al. (2006) J. Biol. Chem. June 23, epub.

G. Cell Cycle Control

The stage of the cell cycle in which DNA damage occurs might also be of importance, as conservative homologous recombination requires a homologous DNA template. It has been suggested that the sister chromatid, present in the late S, G₂ and M phases of the cell cycle, would favor conservative homologous recombination during these phases of the cell cycle (Hendrickson (1997) Am. J. Hum. Genet. 61 795-800). Error-free, conservative homologous recombination involves strand invasion and requires a homologous DNA template, and therefore it is generally believed that this type of repair occurs preferentially in the late S, G₂ and M phases of the cell cycle, when the sister chromatid is available (Saleh-Gohari and Helleday (2005) Mutat. Res. 577(1-2):275-83). In one embodiment of the present invention, the efficiency of homologous recombination is increased by modulating (increasing or decreasing) the level of cell cycle proteins involved in the progression toward or maintenance in the late S, G₂ and M phases of the cell cycle, or by decreasing the level of proteins involved in the progression toward or maintenance in the G₁ phase of the cell cycle. The cyclins and cyclin-dependent kinases that are central to cell cycle control are well known in the art and are described in Lodish et al., eds. (2004) Molecular Biology of the Cell 5^{th} edition (WH Freeman, New York, N.Y.).

F. Facilitating Entry and Stabilization of Polynucleotides

In other embodiments, the level and/or activity of at least one gene product which (a) facilitates entry of a polynucleotide (such as the integrating DNA) into the cell, (b) facilitates transport of the polynucleotide to the homologous genomic locus in the nucleus, (c) increases the resistance of the polynucleotide to degradation and (d) increases the entry of the polynucleotide into the homologous recombination pathway can be modulated (increased or decreased). For example, transiently inhibiting one or more gene involved in cell wall integrity my facilitate entry under certain transfect ion conditions, while the inhibition of one or more gene involved with chromatin assembly/stability/condensation could facilitate “pairing” of the heterologous DNA with the genomic locus.

IV. Modulating the Level of a Gene Product

A. Increasing the Level of Gene Products

As discussed above, in specific methods and compositions of the invention, the level (concentration and/or activity) of a polynucleotide or polypeptide. By “increases” or “increasing” the expression level of a polynucleotide or a polypeptide encoded thereby is intended to mean, the polynucleotide or polypeptide level of the sequence is statistically higher than the polynucleotide level or polypeptide level of the same target sequence in an appropriate control. In particular embodiments, increasing the polynucleotide level and/or the polypeptide level according to the presently disclosed subject matter results in greater than a 95%, 90%, 80%, 70%, 60%, 50%, 40%, 30%, 20%, 10%, or 5% of the polynucleotide level, or the level of the polypeptide encoded thereby, when compared to an appropriate control. In other embodiments, increasing the polynucleotide level and/or the polypeptide level results in greater than about a 3%-15%, 10%-25%, 20% to 35%, 30% to 45%, 40%-55%, 50%-65%, 60%-75%, 70%-85%, 80%-95%, 90%-105%, 100%-115%, 105%-120%, 115%-130%, 125%-150%, 140%-160%, 155%-220% or greater level of the polynucleotide level, or the level of the polypeptide encoded thereby, when compared to an appropriate control. Methods to assay for the level of the RNA transcript, the level of the encoded polypeptide, or the activity of the polynucleotide or polypeptide are discussed elsewhere herein.

An increase in the level and/or activity of a polypeptide of interest can be achieved by providing to the cell one of the polypeptides. As discussed elsewhere herein, many methods are known the art for providing a polypeptide to a cell including, but not limited to, direct introduction of the polypeptide into the cell or introducing into the cell (transiently or stably) a polynucleotide construct encoding a polypeptide having the desired activity. It is also recognized that the methods of the invention may employ a polynucleotide that is not capable of directing the expression of a protein or an RNA. Thus, the level and/or activity of the desired polypeptide may be increased by altering the gene encoding the polypeptide or its promoter. Therefore genetically engineered cells that carry mutations in the gene, where the mutations increase expression of the gene or increase the activity of the encoded polypeptide are provided.

In the methods and compositions of the invention, the increase in gene product level and/or activity can be transient in nature. Methods that allow for a transient increase in the level and/or activity of a polypeptide of interest are disclosed elsewhere herein.

B. Reducing the Level of a Gene Product

Methods and compositions are provided which reduce the level of expression (concentration and/or activity) of a target polynucleotide. As used herein, a “target sequence” comprises any sequence that one desires to decrease the level of expression. The target sequence includes sequences which both encode and do not encode polypeptides. By “reduces” or “reducing” the expression level of a polynucleotide or a polypeptide encoded thereby is intended to mean, the polynucleotide or polypeptide level of the target sequence is statistically lower than the polynucleotide level or polypeptide level of the same target sequence in an appropriate control. In particular embodiments, reducing the polynucleotide level and/or the polypeptide level of the target sequence according to the presently disclosed subject matter results in less than 95%, less than 90%, less than 80%, less than 70%, less than 60%, less than 50%, less than 40%, less than 30%, less than 20%, less than 10%, or less than 5% of the polynucleotide level, or the level of the polypeptide encoded thereby, of the same target sequence in an appropriate control. In other embodiments, reducing the polynucleotide level and/or the polypeptide level of the target sequence results in less than about 3%-15%, 10%-25%, 20% to 35%, 30% to 45%, 40%-55%, 50%-65%, 60%-75%, 70%-90%, 70% to 80%, 70%-85%, 80%-95%, 90%-100% level of the polynucleotide level, or the level of the polypeptide encoded thereby, when compared to an appropriate control. Methods to assay for the level of the RNA transcript, the level of the encoded polypeptide, or the activity of the polynucleotide or polypeptide are discussed elsewhere herein.

In the methods and compositions of the invention, the decrease in gene product level and/or activity can be transient in nature. Methods that allow for a transient decrease in the level and/or activity of a polypeptide of interest are disclosed elsewhere herein.

A. Silencing Elements

By “silencing element” is intended a polynucleotide which when expressed or introduced into a host cell is capable of reducing or eliminating the level or expression of a target polynucleotide or the polypeptide encoded thereby. The silencing element employed can reduce or eliminate the expression level of the target sequence by influencing the level of the target RNA transcript or, alternatively, by influencing translation and thereby affecting the level of the encoded polypeptide. Methods to assay for functional silencing elements that are capable of reducing or eliminating the level of a sequence of interest are disclosed elsewhere herein. Silencing elements can include, but are not limited to, a sense suppression element, an antisense suppression element, an siRNA, an shRNA, a protein nucleic acid (PNA) molecule, a miRNA, a hairpin suppression element, or any precursor thereof.

Thus, a silencing element can comprise a template for the transcription of a sense suppression element, an antisense suppression element, a siRNA, a shRNA, a miRNA, or a hairpin suppression element; an RNA precursor of an antisense RNA, a siRNA, an shRNA, a miRNA, or a hairpin RNA; or, an the active antisense RNA, siRNA, shRNA, miRNA, or hairpin RNA. Methods of introducing the silencing element into the cell may vary depending on which form (DNA template, RNA precursor, or active RNA) is introduced into the cell. When the silencing element comprises a DNA molecule encoding an antisense suppression element, a siRNA, an shRNA, a miRNA, or a hairpin suppression element an interfering RNA, it is recognized that the DNA can be designed so that it is transiently present in a cell or stably incorporated into the genome of the cell. Such methods are discussed in further detail elsewhere herein.

The silencing element can reduce or eliminate the expression level of a target sequence by influencing the level of the target RNA transcript, by influencing translation and thereby affecting the level of the encoded polypeptide, or by influencing expression at the pre-transcriptional level (i.e., via the modulation of chromatin structure, methylation pattern, etc., to alter gene expression). See, for example, Verdel et al. (2004) Science 303:672-676; Pal-Bhadra et al. (2004) Science 303:669-672; Allshire (2002) Science 297:1818-1819; Volpe et al. (2002) Science 297:1833-1837; Jenuwein (2002) Science 297:2215-2218; and Hall et al. (2002) Science 297:2232-2237. Methods to assay for functional interfering RNA that are capable of reducing or eliminating the level of a sequence of interest are disclosed elsewhere herein.

Any region or multiple regions of a target polynucleotide can be used to design a domain of the silencing element that shares sufficient sequence identity to allow the silencing element to decrease the level of the target polynucleotide. For instance, the silencing element can be designed to share sequence identity to the 5′ untranslated region of the target polynucleotide(s), the 3′ untranslated region of the target polynucleotide(s), exonic regions of the target polynucleotide(s), intronic regions of the target polynucleotide(s), and any combination thereof.

The ability of a silencing element to reduce the level of the target polynucleotide may be assessed directly by measuring the amount of the target transcript using, for example, Northern blots, nuclease protection assays, reverse transcription (RT)-PCR, real-time RT-PCR, microarray analysis, and the like. Alternatively, the ability of the silencing element to reduce the level of the target polynucleotide may be measured directly using a variety of affinity-based approaches (e.g., using a ligand or antibody that specifically binds to the target polypeptide) including, but not limited to, Western blots, immunoassays, ELISA, flow cytometry, protein microarrays, and the like. In still other methods, the ability of the silencing element to reduce the level of the target polynucleotide can be assessed indirectly, e.g., by measuring a functional activity of the polypeptide encoded by the transcript or by measuring a signal produced by the polypeptide encoded by the transcript.

Non-limiting examples of silencing elements are discussed in further detail below.

i. Double Stranded RNA Silencing Elements

In one embodiment, the silencing element comprises or encodes a double stranded RNA molecule. As used herein, a “double stranded RNA” or “dsRNA” refers to a polyribonucleotide structure formed either by a single self-complementary RNA molecule or a polyribonucleotide structure formed by the expression of least two distinct RNA strands. Accordingly, as used herein, the term “dsRNA” is meant to encompass other terms used to describe nucleic acid molecules that are capable of mediating RNA interference or gene silencing, including, for example, small RNA (sRNA), short-interfering RNA (siRNA), double-stranded RNA (dsRNA), micro-RNA (miRNA), hairpin RNA, short hairpin RNA (shRNA), and others. See, for example, Meister and Tuschl (2004) Nature 431:343-349 and Bonetta et al. (2004) Nature Methods 1:79-86.

In specific embodiments, at least one strand of the duplex or double-stranded region of the dsRNA shares sufficient sequence identity or sequence complementarity to the target polynucleotide to allow for the dsRNA to reduce the level of expression of the target sequence. As used herein, the strand that is complementary to the target polynucleotide is the “antisense strand,” and the strand homologous to the target polynucleotide is the “sense strand.”

In one embodiment, the dsRNA comprises a hairpin RNA. A hairpin RNA comprises an RNA molecule that is capable of folding back onto itself to form a double stranded structure. Multiple structures can be employed as hairpin elements. For example, the hairpin RNA molecule that hybridizes with itself to form a hairpin structure can comprises a single-stranded loop region and a base-paired stem. The base-paired stem region can comprise a sense sequence corresponding to all or part of the target polynucleotide and further comprises an antisense sequence that is fully or partially complementary to the sense sequence. Thus, the base-paired stem region of the silencing element can determine the specificity of the silencing. See, for example, Chuang and Meyerowitz (2000) Proc. Natl. Acad. Sci. USA 97:4985-4990, herein incorporated by reference. A transient assay for the efficiency of hpRNA constructs to silence gene expression in vivo has been described by Panstruga et al. (2003) Mol. Biol. Rep. 30:135-140, herein incorporated by reference.

ii. siRNA Silencing Elements

A “short interfering RNA” or “siRNA” comprises an RNA duplex (double-stranded region) and can further comprises one or two single-stranded overhangs, e.g., 3′ or 5′ overhangs. The duplex can be approximately 19 base pairs (bp) long, although lengths between 17 and 29 nucleotides, including 17, 18, 19, 20, 21, 22, 23, 24, 25, 26, 27, 28, and 29 nucleotides, can be used. In other embodiments, the length can be from about 17-2000 nucleotides including, for example, between about 30-70, about 65-95, about 90-120, about 115-200, about 175-250, about 195-350, about 300-500, about 400-700, about 600-900, about 800-1200, about 90°-1500, about 1400-1700, about 1600-1900, or about 1800-2000 nucleotides or greater can be used. An siRNA can be formed from two RNA molecules that hybridize together or can alternatively be generated from a single RNA molecule that includes a self-hybridizing portion. The duplex portion of an siRNA can include one or more bulges containing one or more unpaired and/or mismatched nucleotides in one or both strands of the duplex or can contain one or more noncomplementary nucleotide pairs. One strand of an siRNA (referred to herein as the antisense strand) includes a portion that hybridizes with a target transcript. In certain embodiments, one strand of the siRNA (the antisense strand) is precisely complementary with a region of the target transcript over at least about 17 nucleotides, 18 nucleotides, 19 nucleotides, 20 nucleotides or more meaning that the siRNA antisense strand hybridizes to the target transcript without a single mismatch (i.e., without a single noncomplementary base pair) over that length. In other embodiments, one strand of the siRNA (the antisense strand) is precisely complementary with a region of the target transcript over at least about 17-35 nucleotides, 20-40 nucleotides, 25-60 nucleotides or more meaning that the siRNA antisense strand hybridizes to the target transcript without a single mismatch (i.e., without a single noncomplementary base pair) over that length. In other embodiments, one or more mismatches between the siRNA antisense strand and the targeted portion of the target transcript can exist. In embodiments in which perfect complementarity is not achieved, any mismatches between the siRNA antisense strand and the target transcript can be located at or near 3′ end of the siRNA antisense strand. For example, in certain embodiments, nucleotides 1-9, 2-9, 2-10, and/or 1-10 of the antisense strand are perfectly complementary to the target. In specific embodiments, the siRNA has a 3′ overhang of about 1, 2, 3, 4, or 5 nucleotides.

Considerations for design of effective siRNA molecules are discussed in McManus et al. (2002) Nature Reviews Genetics 3: 737-747 and in Dykxhoorn et al. (2003) Nature Reviews Molecular Cell Biology 4: 457-467. Such considerations include the base composition of the siRNA, the position of the portion of the target transcript that is complementary to the antisense strand of the siRNA relative to the 5′ and 3′ ends of the transcript, and the like. A variety of computer programs also are available to assist with selection of siRNA sequences, e.g., from Ambion (web site having URL www.ambion.com), at web site having URL www.sinc.sunysb.edu/Stu/shilin/rnai.html. Additional design considerations are described in Semizarov et al. Proc. Natl. Acad. Sci. 100: 6347-6352. See, also, Elbashir et al. (2001) Nature 411:494-498, Yang et al. (2002) Proc. Natl. Acad. Sci 99:9942-7, Calegari et al. (2002) Proc Natl Acad Sci 99:14236-40 and Paddison et al. (2002) Proc Natl Acad. Sci 99:1443-8). In specific aspects, the siRNA comprises a 25 nucleotide, blunt ended, Stealth™ siRNA molecule available from Invitrogen Corporation.

iii. Short Hairpin RNA Silencing Elements

The term “short hairpin RNA” or “shRNA” refers to an RNA molecule comprising at least two complementary portions hybridized or capable of hybridizing to form a double-stranded (duplex) structure sufficiently long to mediate RNAi (generally between approximately 17 and 30 nucleotides in length, including 17, 18, 19, 20, 21, 22, 23, 24, 25, 26, 27, 28, 29, 30, 40, 50, 60, 70, 80, 90, 100, 125, 150, 200, 300, 400 or more nucleotides in length, and in some embodiments, typically at least 19 base pairs in length), and at least one single-stranded portion, typically between approximately 1 and 25 or 4 to 25 nucleotides in length that forms a loop connecting the two nucleotides that form the base pair at one end of the duplex portion. In other embodiments, the sequence is about 17-30, 25-35, 30-40, 40-50, 60-100, 100-200, 200-300, 300-400 or more nucleotides in length. The duplex portion can, but does not require, one or more bulges consisting of one or more unpaired nucleotides. In specific embodiments, the shRNAs comprise a 3′ overhang. Thus, shRNAs are precursors of siRNAs and are, in general, similarly capable of inhibiting expression of a target transcript. See, for example, Paddison et al. (2002) Genes & Dev 16:948-958.

In particular, RNA molecules having a hairpin (stem-loop) structure can be processed intracellularly by Dicer to yield an siRNA structure referred to as short hairpin RNAs (shRNAs), which contain two complementary regions that hybridize to one another (self-hybridize) to form a double-stranded (duplex) region referred to as a stem, a single-stranded loop connecting the nucleotides that form the base pair at one end of the duplex, and optionally an overhang, e.g., a 3′ overhang. The stem can comprise about 19, 20, or 21 bp long, though shorter and longer stems (e.g., up to about 29 nt) also can be used. The loop can comprise about 1-20, including 1, 2, 3, 4, 5, 6, 7, 8, 9, 10, 11, 12, 13, 14, 15, 16, 17, 18, 19, 20 nt, about 4-10, or about 6-9 nt. The overhang, if present, can comprise approximately 1-20 nt or approximately 2-10 nt. The loop can be located at either the 5′ or 3′ end of the region that is complementary to the target transcript whose inhibition is desired (i.e., the antisense portion of the shRNA).

Although shRNAs contain a single RNA molecule that self-hybridizes, it will be appreciated that the resulting duplex structure can be considered to comprise sense and antisense strands or portions relative to the target mRNA and can thus be considered to be double-stranded. It will therefore be convenient herein to refer to sense and antisense strands, or sense and antisense portions, of an shRNA, where the antisense strand or portion is that segment of the molecule that forms or is capable of forming a duplex with and is complementary to the targeted portion of the target polynucleotide, and the sense strand or portion is that segment of the molecule that forms or is capable of forming a duplex with the antisense strand or portion and is substantially identical in sequence to the targeted portion of the target transcript. In general, considerations for selection of the sequence of the antisense strand of an shRNA molecule are similar to those for selection of the sequence of the antisense strand of an siRNA molecule that targets the same transcript.

iv. MicroRNA Silencing Elements

In one embodiment, the silencing element comprises an miRNA. “MicroRNAs” or “miRNAs” are regulatory agents comprising about 19 ribonucleotides which are highly efficient at inhibiting the expression of target polynucleotides. See, for example, Saetrom et al. (2006) Oligonucleotides 16:115-144, Wang et al. (2006) Mol. Cell. 22:553-60, Davis et al. (2006) Nucleic Acid Research 34:2294-304, Pasquinelli (2006) Dev. Cell 10:419-24, all of which are herein incorporated by reference. For miRNA interference, the silencing element can be designed to express a dsRNA molecule that forms a hairpin structure containing a 19-nucleotide sequence that is complementary to the target polynucleotide of interest. The miRNA can be synthetically made, or transcribed as a longer RNA which is subsequently cleaved to produce the active miRNA. Specifically, the miRNA can comprise 19 nucleotides of the sequence having homology to a target polynucleotide in sense orientation and 19 nucleotides of a corresponding antisense sequence that is complementary to the sense sequence.

It is recognized that various forms of an miRNA can be transcribed including, for example, the primary transcript (termed the “pri-miRNA”) which is processed through various nucleolytic steps to a shorter precursor miRNA (termed the “pre-miRNA”); the pre-miRNA; or the final (mature) miRNA is present in a duplex, the two strands being referred to as the miRNA (the strand that will eventually basepair with the target) and miRNA*. The pre-miRNA is a substrate for a form of dicer that removes the miRNA/miRNA* duplex from the precursor, after which, similarly to siRNAs, the duplex can be taken into the RISC complex. It has been demonstrated that miRNAs can be transgenically expressed and be effective through expression of a precursor form, rather than the entire primary form (McManus et al. (2002) RNA 8:842-50). In specific embodiments, 2-8 nucleotides of the miRNA are perfectly complementary to the target. A large number of endogenous human miRNAs have been identified. For structures of a number of endogenous miRNA precursors from various organisms, see Lagos-Quintana et al. (2003) RNA 9(2):175-9; see also Bartel (2004) Cell 116:281-297.

A miRNA or miRNA precursor can share at least about 80%, 85%, 90%, 91%. 92%, 93%, 94%, 95%, 96%, 97%, 98%, 99%, or 100% or about 75%-83%, 83%-89%, 89%-94%, 94%-97%, or 97% to 100% sequence complementarity with the target transcript for a stretch of at least about 15, 16, 17, 18, 19, 20, 21, 22, 23, 24, 25, 26, 27, 28, 29, or 30 nucleotides or about 15-20, 20-23, 23-17, or 27-30 nucleotides. In specific embodiments, the region of precise sequence complementarity is interrupted by a bulge. See, Ruvkun (2001) Science 294: 797-799, Zeng et al. (2002) Molecular Cell 9:1-20, and Mourelatos et al. (2002) Genes Dev 16:720-728.

V. Antisense Silencing Elements

As used herein, an “antisense silencing element” comprises a polynucleotide which is designed to express an RNA molecule complementary to all or part of a target messenger RNA. Expression of the antisense RNA suppression element reduces or eliminates the level of the target polynucleotide. The polynucleotide for use in antisense suppression may correspond to all or part of the complement of the sequence encoding the target polynucleotide, all or part of the complement of the 5′ and/or 3′ untranslated region of the target polynucleotide, all or part of the complement of the coding sequence of the target polynucleotide, or all or part of the complement of both the coding sequence and the untranslated regions of the target polynucleotide. In addition, the antisense suppression element may be fully complementary (i.e., 100% identical to the complement of the target sequence) or partially complementary (i.e., less than 100% identical to the complement of the target sequence) to the target polynucleotide. In specific embodiments, the antisense suppression element comprises at least about 85%, 90%, 91%, 92%, 93%, 94%, 95%, 96%, 97%, 98%, or 99% or about 85%-89%, 89%-92%, 92%-94%, 94%-97%, or 97% to 100% sequence identity to the target polynucleotide. Antisense suppression may be used to inhibit the expression of multiple proteins in the same plant. See, for example, U.S. Pat. No. 5,942,657. Furthermore, the antisense suppression element can be complementary to a portion of the target polynucleotide. Generally, sequences of at least about 25, 50, 100, 200, 300, 400, 450 nucleotides or greater or about 20-50, about 50-100, about 100-200, about 200-300, about 300-400, about 400-450 or greater may be used. Methods for using antisense suppression are described, for example, in Liu et al (2002) Plant Physiol. 129:1732-1743 and U.S. Pat. Nos. 5,759,829 and 5,942,657, each of which is herein incorporated by reference.

vi. Sense Suppression Elements

As used herein, a “sense suppression element” comprises a polynucleotide designed to express an RNA molecule corresponding to at least a part of a target messenger RNA in the “sense” orientation. Expression of the RNA molecule comprising the sense suppression element reduces or eliminates the level of the target polynucleotide or the polypeptide encoded thereby. The polynucleotide comprising the sense suppression element may correspond to all or part of the sequence of the target polynucleotide, all or part of the 5′ and/or 3′ untranslated region of the target polynucleotide, all or part of the coding sequence of the target polynucleotide, or all or part of both the coding sequence and the untranslated regions of the target polynucleotide. In some embodiments, where the sense suppression element comprises all or part of the coding region for the target polynucleotide, the sense suppression element is designed to have homology to the start codon of the polynucleotide so that no protein product will be transcribed. See, for example, Broin et al. (2002) Plant Cell 14:1417-1432; U.S. Pat. No. 5,942,657; Flavell (1994) Proc. Natl. Acad. Sci. USA 91:3490-3496; Jorgensen et al. (1996) Plant Mol. Biol. 31:957-973; Johansen and Carrington (2001) Plant Physiol. 126:930-938; Stoutjesdijk et al (2002) Plant Physiol. 129:1723-1731; Yu et al. (2003) Phytochemistry 63:753-763; and U.S. Pat. Nos. 5,034,323, 5,231,020, 5,283,184, and 5,942,657; each of which is herein incorporated by reference.

Typically, a sense suppression element has substantial sequence identity to the target polynucleotide, optimally greater than about 65% sequence identity, more optimally greater than about 85% sequence identity, about 90%, 91%, 92%, 93%, 94%, 95%, 96%, 97%, 98% or 99% sequence identity or about 85%-90%, about 90% to about 95%, or about 95% to about 100% sequence identity. See, U.S. Pat. Nos. 5,283,184 and 5,034,323; herein incorporated by reference. The sense suppression element can be any length so long as it does not interfere with intron splicing and allows for the suppression of the targeted sequence. The sense suppression element can be, for example, 15, 30, 50, 100, 150, 200, 250, 300, 350, 400, 450, 500, 600, 700, 900 or longer or about 15-30, 30-50, 50-100, 100-150, 150-200, 200-250, 250-300, 300-350, 350-400, 400-450, 450-500, 500-600, 600-700, 700-900 or longer.

B. Preparing Silencing Elements

Those of ordinary skill in the art will readily appreciate that a silencing element can be prepared according to any available technique including, but not limited to, chemical synthesis, enzymatic or chemical cleavage in vivo or in vitro, template transcription in vivo or in vitro, or combinations of the foregoing. For example, siRNA, can be produced in vitro by chemical synthesis (Hohjoh (2002) FEBS Lett 521: 195-199, hydrolysis of dsRNA (Yang et al. (2002) Proc Natl Acad Sci USA 99: 9942-9947), by in vitro transcription with T7 RNA polymerase (Donzeet et al. (2002) Nucleic Acids Res 30: e46; Yu et al. (2002) Proc Natl Acad Sci USA 99: 6047-6052), by hydrolysis of double-stranded RNA using a nuclease such as E. coli RNase III (Yang et al. (2002) Proc Natl Acad Sci USA 99: 9942-9947), or by in vivo transcription. See also, WO2005070044, herein incorporated by reference. In addition, Nucleic acids that mediate suppression may be synthesized in vitro using methods to produce oligonucleotides and other nucleic acids as described in published international Patent Application No. WO 02/061034; U.S. Provisional Patent Application No. 60/254,510, filed Dec. 8, 2000; U.S. Provisional Patent Application No. 60/326,092, filed Sep. 28, 2001; U.S. patent application Ser. No. 10/014,128, filed Dec. 7, 2001; and U.S. Provisional Patent Application No. 60/520,946, filed Nov. 17, 2003; the disclosures of which applications are incorporated by reference herein in their entireties.

Additional methods for oligonucleotide synthesis of silencing elements are also known, including conventional solid-phase synthesis along with methods that employ phosphorothioates and alkylated derivatives. See, for example, U.S. Pat. No. 4,517,338; U.S. Pat. No. 4,458,066; Lyer et al. (1999) Curr Opin Mol.: 344-358; Verma et al. (1998) Arans Rev Biochem. 67:99-134; Pfleiderer et al. (1996) Acta Biochim Pol. 43: 37-44,1996; Warren et al. (1995) Mol. Biotechnol. 4: 179-199; De Mesmaeker et al. (1995) Curr Opin Struct Biol. 5:343-355; Charubala et al. (1994) Prog Mol Subcell Biol. 14: 114-138; and, Caruthers et al. (1983) Gene Amplif Anal. 3:1-26.

Methods for preparing and purifying various silencing elements in vitro including, siRNA, shRNA, stRNAs, antisense RNAs, and miRNAs are also disclosed in WO2005/012487, herein incorporated by reference. Briefly, WO2005/012487 discloses various DICER reactions and RNase III reactions which can be used to cleave dsRNA substrates and generate siRNAs, as well as, various methods to purify the resulting cleaved product. See also, US Publication 2006/0009409, herein incorporated by reference.

In vitro transcription may be performed using a variety of available systems including the T7, SP6, and T3 promoter/polymerase systems (e.g., those available commercially from Promega, Clontech, New England Biolabs, and the like). Vectors including the T7, SP6, or T3 promoter are well known in the art and can readily be modified to direct transcription of silencing elements. When silencing elements are synthesized in vitro the strands may be allowed to hybridize before introducing them into a cell. As noted above, silencing elements can be introduced into a cell as a single RNA molecule including self-complementary portions (e.g., an shRNA that can be processed intracellularly to yield an siRNA), or as two strands hybridized to one another. In other embodiments, the silencing elements employed are transcribed in vivo. As discussed elsewhere herein, regardless of if the silencing element is transcribed in vivo or in vitro, in either scenario, a primary transcript can be produced which is then processed (e.g., by one or more cellular enzymes) to generate the interfering RNA that accomplishes gene inhibition.

It is recognized that the silencing elements can comprise one or more base modifications, sugar modifications, or backbone modifications or the like. Exemplary base modifications include, for example, phosphorothioate linkages or 2′-deoxy-2′fluorouridine. See, for example, Braasch et al. (2003) Biochemistry 42:7967-75. Additional derivatives of purines and pyrimidines are known, including but not limited to, aziridinylcytosine, 4-acetylcytosine, 5-fluorouracil,5-bromouracil, 5-carboxymethylaminomethyl-2-thiouracil, 5-carboxymethylaminomethyluracil, inosine (and derivatives thereof), N6-isopentenyladenine, 1-methyladenine, 1-methylpseudouracil, 1-methylguanine, 1-methylinosine, 2,2-dimethylguanine, 2-methyladenine, 2-methylguanine, 7-methylguanine, 3-methylcytosine, 5-methylcytosine(5MC), N6-methyladenine, 5-methylaminomethyluracil, 5-methoxyaminomethyl-2-thiouracil, beta-D-mannosylqueosine, 5-methoxyuracil, 2-methylthio-N-6-isopentenyladenine, uracil-5-oxyacetic acid methylester, pseudouracil, queosine, 2-thiocytosine, 5-methyl-2-thiouracil, 2-thiouracil, 4-thiouracil, 5-methyluracil, uracil-5-oxyacetic acid, and 2,6-diaminopurine. In addition to nucleic acids that incorporate one or more of such base derivatives, nucleic acids having nucleotide residues that are devoid of a purine or a pyrimidine base may also be included in oligonucleotides and other nucleic acids.

Non-limiting sugar modifications include, for example, substitution at the 2′-position of the furanose residue enhances nuclease stability. An exemplary, but not exhaustive list, of modified sugar residues includes 2′ substituted sugars such as 2′-O-methyl-, 2′-O-alkyl,2′-O-allyl, 2′-S-alkyl, 2′-S-allyl, 2′-fluoro-, 2′-halo, or 2′-azido-ribose, carbocyclic sugar analogs, alpha-anomeric sugars, epimeric sugars such asarabinose, xyloses or lyxoses, pyranose sugars, furanose sugars, sedoheptuloses, acyclic analogs and abasic nucleoside analogs such as methyl riboside, ethyl riboside orpropylriboside.

The silencing elements can also comprise one or more backbone modification. For example, chemically modified backbones include phosphorothioates, chiral phosphorothioates, phosphorodithioates, phosphotriesters, aminoalkylphos-photriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotri-esters, and boranophosphates having normal 3′-5′linkages, 2′-5′linked analogs of these, and those having inverted polarity wherein the adjacent pairs of nucleoside units are linked 3′-5′ to 5′-3′ or 2′-5′ to 5′-2′. Chemically modified backbones that do not contain a phosphorus atom have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages, including without limitation morpholino linkages; siloxane backbones; sulfide, sulfoxide and sulfone backbones formacetyl and thioformacetyl backbones; methyleneformacetyl and thioformacetyl backbones; alkene containing backbones; sulfamate backbones; methyleneimino and methylenehydrazino backbones; sulfonate and sulfonamide backbones; and amide backbones.

V. Homologous Recombination Cassettes

As used herein, a “homologous recombination cassette” comprises a polynucleotide having at least one region with sufficient sequence identity to a predetermined target site that allows for the integration of the cassette at the predetermined locus via a homologous recombination event. In specific embodiments, the homologous recombination cassette further comprises a polynucleotide of interest.

Various homologous recombination cassettes are known. For example, a homologous recombination cassette can comprise an “insertion cassette.” An insertion homologous recombination cassette comprises a single region sharing sufficient sequence identity to a target site which promotes a single homologous recombination cross-over event. In specific embodiments, the insertion cassette further comprises a polynucleotide of interest. As only a single cross-over event occurs, the entire insertion cassette and the plasmid/vector it is contained in, is integrated at the target site. Such insertion cassettes are generally contained on circular vectors/plasmids. See, U.S. Publications 2003/0131370 and 2003/0157076 and 2003/0188325 and 2004/0107452 and Thomas et al. (1987) Cell 51:503-512.

In other embodiments, the homologous recombination cassette comprises a “replacement vector.” Replacement homologous recombination cassettes comprise a first and a second region having sufficient sequence identity to a corresponding first and second region of a target site in a eukaryotic cell. A double homologous recombination cross-over event occurs and any polynucleotide internal to the first and second region is integrated at the target site (i.e., homologous recombination between the first region of homology of the cassette and the corresponding first region of the target site and homologous recombination between the second region of homology of the recombination cassette and the corresponding second region of the target site).

As used herein, a “target site” comprises a pre-determined location in a DNA sequence into which integration of exogenous DNA is desired. When employing a replacement homologous recombination cassette, the target site is defined by flanking target sequences that allow for a homologous recombination with the corresponding sequences of the replacement homologous recombination expression cassette.

It is recognized that the homologous recombination cassette can be designed for a target site that is endogenous or heterologous to the host cell. In addition, the target site can be present on a chromosome or found extrachromosomally in the host cell. In addition, the target site (either the first and/or second region of the target site) can be located in any segment of DNA in the host cell, including, but not limited to, coding sequence, 5′ UTR, 3′UTR, non-coding sequence, intron, exons, regulatory regions, promoters, enhancers, etc.

When using a replacement homologous expression cassette, the first and the second regions of the target site can be contiguous with one another or non-contiguous with respect to one another. For example, the first and the second regions that correspond to the target site replace an exon of a gene of interest. In such an embodiment, the homologous recombination cassette comprises a first region sharing sufficient sequence identity an intron 5′ to the exon of interest, a replacement exon, and the second region sharing sufficient sequence identity to an intron 3′ to the exon of interest. Following a double cross over homologous recombination event between the target site and the homologous recombination cassette, the exon in the homologous recombination cassette is exchanged with the original exon present in the cell.

The regions of sequence identity between the first and the second regions of the homologous recombination cassette and the corresponding regions of the target site can be of any length so long as the are capable of promoting homologous recombination. For example, the first and/or the second regions of the homologous recombination cassette can be less than or greater than about 15000, 12000, 10000, 8000, 6000, 4000, 2000, 1000, 800, 600, 400, 200, 100, 50, 25 nucleotides or less. In other embodiments, the regions that share sufficient sequence identity comprise between about 25 to 50, about 50 to 100, about 100 to about 400, about 400 to about 700, about 700 to about 1200, about 1200 to about 1700, about 1700 to about 2000, about 2000 to about 5000 nt, about 5000 to about 10000 nt, about 10000 to 15000.

The “first” region of the target site and the corresponding “first” region of the homologous recombination cassette need only sufficient sequence identity to allow for a homologous recombination event. Similarly, the “second” region of the target site and the corresponding “second” region of the homologous recombination cassette need only sufficient sequence identity to allow for a homologous recombination event. In specific embodiments, the first region of the target site and the first region of the recombination cassette and/or the second region of the target site and the second region of the recombination site have 100% sequence identity to one another. Alternatively, these regions may share partial sequence identity to each other so long as they are capable of undergoing a homologous recombination event. The amount of sequence identity between the region of the target site and the corresponding region of the cassette can be calculated as a percentage of the entire region. Thus, the region of the target site and the corresponding region of the recombination cassette generally share at least 80%, 85%, 90%, 91%, 92%, 93%, 94%, 95%, 96%, 97%, 98%, 99%, up to and including 100% sequence identity. It is recognized that level of percent identity shared between the arm of the homologous recombination cassette and the target site will vary depending on the length of the arm.

Methods of designing homologous recombination cassettes can be found, for example, in WO2006057466, US2006127978, US2006051776, Scheerer et al. (1994) Mol Cell Biol. 14(10) 6663-6673, Thomas et al. (1987) Cell 51 (3) 503-512; Hasty et al. (1991) Mol Cell Biol. 11 (11); 5586-5591; and, Lu et al. (2003) Blood 102 (4) 1531-1533. The design of the cassette and the makers employed will influence how cells having the homologous recombination event are detected and/or selected. In one embodiment, the homologous recombination cassette comprises a positive-negative selection (PNS) vector. Briefly, such cassettes can comprises in the 5′ to 3′ or 3′ to 5′ direction, a first region having sufficient sequence identity to a corresponding first region of a target site, a polynucleotide of interest, a positive selection marker, a second region having sufficient sequence identity to a corresponding second region of the target site, and a negative section marker. With this cassette, the negative selection marker is stably integrated into a cell only via a non-homologous recombination event. PNS vectors are described in more detail in, for example, U.S. Pat. Nos. 5,464,764, 5,487,992, 5,627,059, 5,631,153, 6,204,061, 6,689,610, each of which is herein incorporated by reference. Such vectors allow one to select for homologous recombination events.

The homologous recombination cassette can further comprise multiple cloning sites to allow for the insertion of the polynucleotide of interest between the flanking regions. The polynucleotide of interest contained in the homologous recombination cassette can comprises, but is not limited to, a promoter element, a therapeutic gene, a marker, a control region, a trait-producing fragment, a nucleic acid fragment to accomplish gene disruption, etc. Moreover, the polynucleotide of interest employed in the homologous recombination cassette can encode or modulate the activity of any polynucleotide or polypeptide including those having either medical or industrial application, such as hormones, cytokines, enzymes, coagulation factors, carrier proteins, receptors, regulatory proteins, structural proteins, transcription factors, antigens, antibodies and the like. Such constructs can be used to generate either knock-out or knock-in cells. In knock-out cells the functioning of a particular targeted native gene is disrupted or suppressed in the genome of the cell.

In specific embodiments, the polynucleotide of interest is in an expression cassette. In more specific embodiments, the polynucleotide of interest is operably linked to a promoter. One of skill in the art will be able to select the appropriate promoter for the particular polynucleotide of interest that is employed. In still further embodiments, the homologous recombination cassette can further comprise a marker sequence (including, for example, positive or negative selection makers) which can be employed to identify cells which have undergone the homologous recombination event. Non-limiting markers and promoters that can be employed in the cassette are disclosed elsewhere herein.

VI. Expression Cassettes and Host Cells

An expression cassette comprises one or more regulatory sequences, selected on the basis of the cells to be used for expression, operably linked to the desired polynucleotide. “Operably linked” is intended to mean that the desired polynucleotide (i.e., a DNA encoding a silencing element, DNA encoding a polypeptide that increases homologous recombination activity, DNA that encodes a sequence that decreases non-homologous recombination, selectable markers, etc.) is linked to the regulatory sequence(s) in a manner that allows for expression of the nucleotide sequence (e.g., in an in vitro transcription/translation system or in a cell when the expression cassette or vector is introduced into a cell). “Regulatory sequences” include promoters, enhancers, and other expression control elements (e.g., polyadenylation signals). See, for example, Goeddel (1990) in Gene Expression Technology: Methods in Enzymology 185 (Academic Press, San Diego, Calif.). Regulatory sequences include those that direct constitutive expression of a nucleotide sequence in many types of host cells, those that direct expression of the nucleotide sequence only in certain host cells (e.g., tissue-specific regulatory sequences), or those that direct expression of the polynucleotide in the presence of an appropriate inducer (inducible promoter). It will be appreciated by those skilled in the art that the design of the expression cassette can depend on such factors as the choice of the host cell to be transformed, the level of expression of the polynucleotide that is desired, and the like. Such expression cassettes typically include one or more appropriately positioned sites for restriction enzymes, to facilitate introduction of the nucleic acid into a vector.

It will further be appreciated that appropriate promoter and/or regulatory elements can readily be selected to allow expression of the relevant transcription units in the cell of interest. In certain embodiments, the promoter utilized to direct intracellular expression of a silencing element is a promoter for RNA polymerase III (Pol III). References discussing various Pol III promoters, include, for example, Yu et al. (2002) Proc. Natl. Acad. Sci. 99(9), 6047-6052; Sui et al. (2002) Proc. Natl. Acad. Sci. 99(8), 5515-5520 (2002); Paddison et al. (2002) Genes and Dev. 16, 948-958; Brummelkamp et al. (2002) Science 296, 550-553; Miyagashi (2002) Biotech. 20, 497-500; Paul et al. (2002) Nat. Biotech. 20, 505-508; Tuschl et al. (2002) Nat. Biotech. 20, 446-448. According to other embodiments, a promoter for RNA polymerase I, e.g., a tRNA promoter, can be used. See McCown et al. (2003) Virology 313(2):514-24; Kawasaki (2003) Nucleic Acids Res. 31 (2):700-7.

The regulatory sequences can also be provided by viral regulatory elements. For example, commonly used promoters are derived from polyoma, Adenovirus 2, cytomegalovirus, and Simian Virus 40. For other suitable expression systems for both prokaryotic and eukaryotic cells, see Chapters 16 and 17 of Sambrook et al. (1989) Molecular Cloning: A Laboratory Manual (2d ed., Cold Spring Harbor Laboratory Press, Plainview, N.Y.). See, Goeddel (1990) in Gene Expression Technology. Methods in Enzymology 185 (Academic Press, San Diego, Calif.).

Various constitutive promoters are known. For example, in various embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest. Promoters which may be used include, but are not limited to, the long terminal repeat as described in Squinto et al. (1991) Cell 65:1 20); the SV40 early promoter region (Bernoist and Chambon (1981) Nature 290:304 310), the CMV promoter, the M-MuLV 5′ terminal repeat the promoter contained in the 3′ long terminal repeat of Rous sarcoma virus (Yamamoto et al. (1980) Cell 22:787 797), the herpes thymidine kinase promoter (Wagner et al. (1981) Proc. Natl. Acad. Sci. U.S.A. 78:144 1445), the regulatory sequences of the metallothionine gene (Brinster et al. (1982) Nature 296:39 42); the following animal transcriptional control regions, which exhibit tissue specificity and have been utilized in transgenic animals: elastase I gene control region which is active in pancreatic acinar cells (Swift et al. (1984) Cell 38:639 646; Ornitz et al. (1986) Cold Spring Harbor Symp. Quant. Biol. 50:399 409; MacDonald, 1987, Hepatology Z:425 515); insulin gene control region which is active in pancreatic beta cells (Hanahan (1985) Nature 315:115 122), immunoglobulin gene control region which is active in lymphoid cells (Grosschedl et al. (1984) Cell 38:647 658; Adames et al (1985) Nature 318:533 538; Alexander et al. (1987) Mol. Cell. Biol. 7:1436 1444), mouse mammary tumor virus control region which is active in testicular, breast, lymphoid and mast cells (Leder et al. (1986) Cell 45:485 495).

Inducible promoters are also known. Non-limiting examples of inducible promoters and their inducer include MT II/Phorbol Ester (TPA) (Palmiter et al. (1982) Nature 300:611) and heavy metals (Haslinger and Karin (1985) Proc. Nat'l Acad. Sci. USA. 82:8572; Searle et al. (1985) Mol. Cell. Biol. 5:1480; Stuart et al. (1985) Nature 317:828; Imagawa et al. (1987) Cell 51:251; Karin et al. (1987) Mol. Cell Biol. 7:606; Angel et al. (1987) Cell 49:729; McNeall et al. (1989) Gene 76:8); MMTV (mouse mammary tumor virus)/Glucocorticoids (Huang et al. (1981) Cell 27:245; Lee et al. (1981) Nature 294:228; Majors and Varmus (1983) Proc. Nat'l Acad Sci. USA. 80:5866; Chandler et al. (1983) Cell 33:489; Ponta et al. (1985) Proc. Nat'l Acad. Sci. USA. 82:1020; Sakai et al. (1988) Genes and Dev. 2:1144); β-Interferon/poly(rI)X and poly(rc) (Tavernier et al. (1983) Nature 301:634); Adenovirus 5 E2/E1A (Imperiale and Nevins (1984) Mol. Cell. Biol. 4:875); c-jun/Phorbol Ester (TPA), H₂O₂; Collagenase/Phorbol Ester (TPA) (Angel et al. (1987) Mol. Cell. Biol. 7:2256); Stromelysin/Phorbol Ester (TPA), IL-1 (Angel et al. (1987) Cell 49:729); SV40/Phorbol Ester (TPA) (Angel et al. (1987) Cell 49:729); Murine MX Gene/Interferon, Newcastle Disease Virus; GRP78 Gene/A23187 (Resendez Jr. et al. (1988) Mol. Cell. Biol. 8:4579); α-2-Macroglobulin/IL-6; Vimentin/Serum (Kunz et al. (1989)Nucl. Acids Res. 17:1121); MHC Class I Gene H-2 kB/Interferon (Blanar et al. (1989) EMBO J. 8:1139); HSP70/E1a, SV40 Large T Antigen (Taylor and Kingston (1990) Mol. Cell. Biol. 10:165; Taylor and Kingston (1990) Mol. Cell. Biol. 10:176; Taylor et al. (1989) J. Biol. Chem. 264:15160); Proliferin/Phorbol Ester-TPA (Mordacq and Linzer (1989) Genes and Dev. 3:760); Tumor Necrosis Factor/PMA (Hensel et al. (1989) Lymphokine Res. 8:347); Thyroid Stimulating Hormone α Gene/Thyroid Hormone (Chatterjee et al. (1989) Proc. Nat'l Acad. Sci. USA. 86:9114); and, Insulin E Box/Glucose.

Such expression cassettes can be contained in a vector which allow for the introduction of the expression cassette into a cell. In specific embodiments, the vector allows for autonomous replication of the expression cassette in a cell or may be integrated into the genome of a cell. Such vectors are replicated along with the host genome (e.g., nonepisomal mammalian vectors). In general, expression vectors of utility in recombinant DNA techniques are often in the form of plasmids (vectors). However, the invention is intended to include such other forms of expression vectors, such as viral vectors (e.g., replication defective retroviruses, adenoviruses, and adeno-associated viruses). See, for example, U.S. Publication 2005214851, herein incorporated by reference.

Any expression cassette can further comprise a selection marker. As used herein, the term “selection marker” comprises any polynucleotide, which when expressed in a cell allows for the selection of the transformed cell with the vector. For example, a selection marker can confer resistance to a drug, a nutritional requirement, or a cytotoxic drug. A selection marker can also induce a selectable phenotype such as fluorescence or a color deposit. A “positive selection marker” allows a cell expressing the marker to survive against a selective agent and thus confers a positive selection characteristic onto the cell expressing that marker. Positive selection marker/agents include, for example, Neo/G418, Neo/Kanamycin, Hyg/Hygromycin, hisD/Histidinol, Gpt/Xanthine, Ble/Bleomycin, HPRT/Hypoxanthine. Other positive selection markers include DNA sequences encoding membrane bound polypeptides. Such polypeptides are well known to those skilled in the art and can comprise, for example, a secretory sequence, an extracellular domain, a transmembrane domain and an intracellular domain. When expressed as a positive selection marker, such polypeptides associate with the cell membrane. Fluorescently labeled antibodies specific for the extracellular domain may then be used in a fluorescence activated cell sorter (FACS) to select for cells expressing the membrane bound polypeptide. FACS selection may occur before or after negative selection.

A “negative selection marker” allows the cell expressing the marker to not survive against a selective agent and thus confers a negative selection characteristic onto the cell expressing the marker. Negative selection marker/agents include, for example, HSV-tk/Acyclovir or Gancyclovir or FIAU, Hprt/6-thioguanine, Gpt/6-thioxanthine, cytosine deaminase/5-fluoro-cytosine, diphtheria toxin or the ricin toxin. See, for example, U.S. Pat. No. 5,464,764, herein incorporated by reference.

In preparing an expression cassette or a homologous recombination cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide.

An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material, or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein of the invention or biologically active portion thereof is recombinantly produced, optimally culture medium represents less than about 30%, 20%, 10%, 5%, or 1% (by dry weight) of chemical precursors or non-protein-of-interest chemicals.

The use of the term “polynucleotide” is not intended to limit the present invention to polynucleotides comprising DNA. Those of ordinary skill in the art will recognize that polynucleotides, can comprise ribonucleotides and combinations of ribonucleotides and deoxyribonucleotides. Such deoxyribonucleotides and ribonucleotides include both naturally occurring molecules and synthetic analogues. The polynucleotides of the invention also encompass all forms of sequences including, but not limited to, single-stranded forms, double-stranded forms, hairpins, stem-and-loop structures, and the like.

VII. Methods of Introducing Sequences into a Cell

Any cell from a eukaryote can be used in the methods and compositions. The cell can be a primary cell, a secondary cell, or a permanent cell. In specific embodiments, the cell is from a mammal, a primate, a human, a domestic animal or an agricultural animal. Non-limiting animals that the cell can be derived from include cattle, sheep, goats, pigs, horses, rabbits, dogs, monkeys, cats, large felines (lions, tigers, etc.), wolves, mice, rats, rabbits, deer, mules, bears, cows, pigs, horses, oxen, zebras, elephants, and so on. The cell can further be from a plant, a worm (e.g., C. elegans), an insect, a fish, a reptile, an amphibian, a bird (including, but not limited to chickens, turkeys, ducks, geese and the like), a marsupial, etc. The cells can be derived from any tissue (diseased or healthy) from any of these organisms. The cell can further comprise a germ cell, an embryonic stem cell, or a primary fibroblast cell from any animal discussed above, including, for example, cells from pigs, mice and humans. Such host cells include cultured cells (in vitro), explants and primary cultures (in vitro and ex vivo).

The methods of the invention involve introducing a polypeptide or polynucleotide into a cell. “Introducing” is intended to mean presenting to the cell the polynucleotide or polypeptide in such a manner that the sequence gains access to the interior of a cell of the plant. The methods of the invention do not depend on a particular method for introducing a sequence into a cell, only that the polynucleotide or polypeptides gains access to the interior of a cell. Methods for introducing polynucleotide or polypeptides into various cell types are known in the art including, but not limited to, stable transformation methods, transient transformation methods, and virus-mediated methods.

“Stable transformation” is intended to mean that the nucleotide construct introduced into a cell integrates into the DNA of the cell and is capable of being inherited by the progeny thereof. “Transient transformation” is intended to mean that a polynucleotide is introduced into the cell and does not integrate into the genome of the cell or a polypeptide is introduced into a cell. Transformation protocols as well as protocols for introducing polypeptides or polynucleotide sequences into cell may vary depending on the type of cell targeted for transformation.

Exemplary art-recognized techniques for introducing foreign polynucleotides into a host cell, include, but are not limited to, calcium phosphate or calcium chloride co-precipitation, DEAE-dextran-mediated transfection, lipofection, particle gun, or electroporation and viral vectors. Suitable methods for transforming or transfecting host cells can be found in U.S. Pat. No. 5,049,386, U.S. Pat. No. 4,946,787; and U.S. Pat. No. 4,897,355, Sambrook et al. (1989) Molecular Cloning: A Laboratory Manual (2d ed., Cold Spring Harbor Laboratory Press, Plainview, N.Y.) and other standard molecular biology laboratory manuals. Various transfection agent can be used in these techniques. Such agent are known, see for example, WO 2005012487. One of skill will recognize that depending on the method by which a polynucleotide is introduced into a cell, the silencing element can be stably incorporated into the genome of the cell, replicated on an autonomous vector or plasmid, or present transiently in the cell.

Viral vector delivery systems include DNA and RNA viruses, which have either episomal or integrated genomes after delivery to the cell. For a review of viral vector procedures, see Anderson (1992) Science 256:808-813; Haddada et al. (1995) Current Topics in Microbiology and Immunology Doerfler and Bohm (eds); and Yu et al. (1994) Gene Therapy 1: 13-26. Conventional viral based systems for the delivery of polynucleotides could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene.

VIII. Methods

Various methods for increasing the homologous recombination activity of a cell are provided. In one embodiment, a method for increasing the efficiency of homologous recombination of a polynucleotide of interest in a eukaryotic cell is provided. The method comprises providing a eukaryotic cell having a decreased level of non-homologous recombination activity, wherein the decreased level of non-homologous recombination activity is transient; and, introducing into the eukaryotic cell a composition comprising a homologous recombination cassette. The polynucleotide of interest is inserted into the target site by a homologous recombination event.

As discussed elsewhere herein, the transient decrease in non-homologous recombination activity can be achieved in many ways. For example, and as discussed elsewhere herein, the silencing elements can be operably linked to an inducible promoter and stably integrated into the host cell, while in other embodiments, the silencing elements are transiently introduced into the cell.

Additional methods of the invention comprise increasing the efficiency of homologous recombination of a polynucleotide of interest in a eukaryotic cell comprising
a) introducing into the eukaryotic cell a polypeptide or a polynucleotide which transiently decreases the level of non-homologous recombination in said eukaryotic cell; and,
b) introducing into the eukaryotic cell a composition comprising a homologous recombination cassette (i.e., an insertion or a replacement homologous recombination cassette), wherein the polynucleotide of interest is inserted into the target site by a homologous recombination event. When the polynucleotide of step (a) comprises a silencing element, the silencing element specifically reduces the level of a gene product that contributes to non-homologous recombination. Non-limiting examples of such gene products are disclosed elsewhere herein.

The transient decrease in non-homologous recombination activity can last for any time period that provides a sufficient window of time for the desired homologous recombination event to occur. Preferably, the decrease in non-homologous activity does not occur for a time period long enough to produce a permanent negative impact the heath and/or viability of the cell. Various methods can be employed which allow one to determine the appropriate time at which the transient decrease in non-homologous recombination activity should begin and the length of time that the non-homologous activity can be decreased. Accordingly, the transient decrease in non-homologous recombination activity can be about 1 hour to 24 hours, about 24 hours to about 36 hours, about 36 hours to about 48 hours, about 48 hours to about 72 hours, or about 72 hours to about 96 hours. In other embodiments, the transient decrease in non-homologous recombination activity can be at least 6-7, 7-8, 8-9, 9-10, or greater. Moreover, the transient decrease in non-homologous recombination activity can occur simultaneously with the introduction of the homologous recombination cassette in the cell, or alternatively, the decrease in non-homologous recombination activity can occur about 5 minutes to 10 minutes, about 5 minutes to 30 minutes, about 30 minutes to about 45 minutes, 45 minutes to about 2 hours, about 2 hours to about 4 hours, about 4 hours to about 6 hours, about 6 hours to about 15 hours, about 15 hours to about 1 day, about 1 day to about 2 days, about 2 days to about 3 days, about 3 days to about 4 days, about 4 days to about 5 days, about 5 days to about 6 days, about 6 days to about 7 days, or more prior to the introduction of the homologous recombination cassette.

Various methods to transiently decrease non-homologous recombination are disclosed elsewhere herein. Briefly, polynucleotides encoding a silencing element can be stably integrated into a cell. The stably integrated silencing element is under the control of an inducible promoter and thus one can transiently decrease non-homologous recombination upon induction of the promoter. In other embodiments, the active forms of the silencing elements are generated in vitro and transiently introduced into a cell.

Following the homologous recombination event, cells having the polynucleotide of interest inserted at the predetermined target site in the host cell can be detected and/or selected. For example, PCR can be used to identify a homologous recombination event. See, for example, Kim et al. (1988) Nucl. Acid. S. Res 16:8887-8903 and Zimmerman et al. (1989) Nature 338:150-153. Additional methods are disclosed in, for example, EP1661992. Positive and negative selection schemes can also be employed.

In specific embodiments, the homologous recombination cassette employed comprises a positive-negative selection (PNS) vector, which are described detail elsewhere herein. In such methods, following the introduction of the homologous recombination cassette into the cell, a positive selection step and a negative selection step is performed. “Positive selection” comprises contacting cells transfected with a PNS vector with an appropriate agent which kills or otherwise selects against cells not containing an integrated positive selection marker. “Negative selection” comprises contacting cells transfected with the PNS vector with an appropriate agent which kills or otherwise selects against cells containing the negative selection marker. Appropriate agents for use with specific positive and negative selection markers are disclosed elsewhere herein.

It is recognized that, if desired, a cell having the polynucleotide of interest inserted only at the pre-determined target site can be detected and selected. Alternatively, cells having the polynucleotide of interest inserted at the predetermined target site and having 1, 2, 3, 4, 5, 6, 7, 8, 10, 15 or more random integrations can be detected and selected.

The efficiency of homologous recombination of a polynucleotide of interest can be further increased by decreasing non-homologous recombination activity and increasing homologous recombination activity by modulating the level and/or activity of a sequence involved in this pathway. In specific embodiments, the modulation of the level and/or activity of sequences that modulate homologous recombination is transient. For example, and as discussed elsewhere herein, a sequence that modulate the homologous recombination can be operably linked to an inducible promoter and stably integrated into the host cell, while in other embodiments, the polypeptide having the desired acidity can be directly introduced into the cell.

The transient modulation in homologous recombination activity can last for any time period that provides a sufficient window of time for the desired homologous recombination event to occur. Preferably, the modulation in homologous activity does not occur for a time period long enough to produce a permanent negative impact the heath and/or viability of the cell. Various methods can be employed which allow one to determine the appropriate time at which the transient modulation in homologous recombination activity should begin and the length of time that the homologous activity can be modulated. Accordingly, the transient modulation in homologous recombination activity can be about 1 hour to 24 hours, about 24 hours to about 36 hours, about 36 hours to about 48 hours, about 48 hours to about 72 hours, or about 72 hours to about 96 hours. In other embodiments, the transient modulation in homologous recombination activity can be at least 6-7, 7-8, 8-9, 9-10, or greater. Various methods to transiently modulate the level of homologous recombination activity are disclosed elsewhere herein.

Accordingly, any method disclosed herein can further comprise introducing in the eukaryotic cell at least one polynucleotide which when expressed in the eukaryotic cell increases homologous recombination activity in the cell or the polypeptide itself can be directly introduced into the cell. For example, prior to introducing the homologous recombination cassette into the cell, the cassette can be coated with a RecA polypeptide, a Rad51 polypeptide, a Rad 54 polypeptide or any combination thereof. The coated homologous recombination cassette can then be introduced into the cell, for example by lipofection.

The methods of the invention can further be combined with one or more regeneration steps to produce tissues or organisms from the genetically modified cell lines (clones). These steps are well known by the person skilled in the art and described in the scientific literature (Thomas et al. (1987) Cell 51: 503-512; Galli-Thaliodoros et al. (1995)J. Immuno. Meth. 181: 1-15; Muller (1999) Mechanisms of Development 82:3-21; Terrihiko et al. (1999) PNAS 96: 1014-1984-14989); and Pfeifer et al. (2002) PNAS: 99: 2140-2145). In specific embodiments, a non-human pluri-cellular organism is selected (by cross-breeding) wherein the organism is homozygous for the desired insertion at the target site. In other embodiments, the method are employed to generate a genetically modified cell line from a stem cell line of an eukaryote organism, such as a non-human totipotent embryonic stem cells (such as mouse ES cells) which could be used for obtaining easily a regeneration of a non-human mammal (such as a rodent, such us a mouse or a rat) from this cell line.

In one embodiment, the transient decrease in non-homologous recombination activity occurs in combination with an increase in the frequency of double-stranded breaks at the predetermined target site. Such methods and compositions thereby promote homologous recombination of the homologous recombination cassette at the target site. The increase in double-stranded breakage at the target site can occur via a targeted or a non-targeted cleavage mechanism. As used herein, a sequence that produces a “targeted” double-stranded break comprises a polypeptide (or a polynucleotide encoding the same) which is designed to target cleavage at the predetermined target site at a higher level or frequency than cleavage at a random site in the genome. As used herein, a sequence that produces a “non-targeted” double stranded break comprises any polypeptide (or polynucleotide encoding the same) which does not target cleavage at the pre-determined target site at a higher level or frequency than cleavage at a random site in the genome.

Various methods can be employed to modulate the level of “targeted” and “non-targeted” double stranded breaks at the target site. For example, a polypeptide (or a polynucleotide encoding the same) can comprise a fusion protein designed to target an endonuclease (i.e., a restriction endonuclease and/or a homing endonuclease) to the predetermined target site. For example, a zinc-finger fusion binding domain operably linked to an appropriate endonuclease can be expressed or provided to the cell. In such embodiments, the target site, or a region near the target site, comprises a binding site for the zinc finger binding domain. The interaction of the zinc finger protein at the target site brings the endonucleases to the target site and thereby promotes the targeted double-stranded break. In specific methods and compositions, the polypeptide (or the polynucleotide encoding the same) which increases the level of targeted double-stranded breaks at the target site does not comprises a zinc-finger binding domain fusion protein.

In other methods and compositions, the level of double-stranded breaks at the target site is increased by a “non-targeted” method. For example, a polypeptide (or a polynucleotide encoding the same) comprising, for example, an endonuclease (i.e., a restriction endonuclease and/or a homing endonuclease) can be provided to a cell. It is recognized that a sequence which increases either a targeted or a non-targeted DNA cleavage event can be heterologous to the cell or native to the cell.

The level and/or activity of various polynucleotide or polypeptides can be modulated to increase DNA double-strand breaks (DSBs) including, for example, sequences involved in cellular processes such as DNA repair, recombination and replication; the early prophase of meiosis, V(D)J recombination or as the result of exposure to DNA damaging agents. See, for example, Manivasakam et al. (2001) Nucleic Acids Research 29(23):4826-4833, herein incorporated by reference in its entirety.

VIII. Kits

Kits are provided which comprise one or more of the components disclosed herein. For example, kits that allow one to decrease the non-homologous recombination activity of a eukaryotic cell and, in specific embodiments, further allow for the targeted stable insertion of a polynucleotide of interest in a eukaryotic cell via a homologous recombination event are provided.

In one embodiment, the kit comprises (a) a compound which when contacted to or introduced into a cell decreases non-homologous recombination, (b) a polynucleotide encoding a polypeptide, or the polypeptide itself, that decreases non-homologous recombination activity, or (c) a polynucleotide encoding a silencing element, wherein the silencing element when introduced into a eukaryotic cell reduces the level of a gene product that contributes to non-homologous recombination, and, increases the homologous recombination activity in the eukaryotic cell. The kit can further comprise one or more polynucleotides comprising a homologous recombination cassette; and/or a polynucleotide which when expressed in said eukaryotic cell increases homologous recombination activity in said eukaryotic cell. In specific embodiments, the polynucleotide which when expressed in said eukaryotic cell further increases homologous recombination activity, is designed to allow for a transient modulation of homologous recombination activity.

In further embodiments, the kit comprises a non-homologous recombination silencing element, including but not limited to, a silencing element for a Ku70 polypeptide, a Xrcc4 polypeptide, a Ligase IV polypeptide, a DNA-dependant protein kinase catalytic subunit (DNA-PKcs) polypeptide or an Artemis nuclease polypeptide. Such a homologous recombination silencing element may be utilized for in vitro transcription or for in vivo transcription, and accordingly, the polynucleotide comprising or encoding the element can further comprise appropriate promoters, selection markers, and any other appropriate regulatory elements as described elsewhere herein. If the silencing element is designed for in-vitro transcription, the kit can further comprise the necessary reagents to carry out the reaction.

Homologous recombination cassette or a polynucleotide engineered to accept a homologous recombination cassette can further be included in a kit of the invention. Such polynucleotides can comprise vectors or plasmids with appropriate regulatory elements and cloning sites to allow for the insertion of sequences which have sufficient sequence identity to the first and/or the second regions of the desired target site. In other embodiments, the kit comprises a homologous recombination cassette comprising the sequences which promote homologous recombination at a predetermined target site, but lacking the polynucleotide of interest, or alternatively, further comprise a polynucleotide of interest.

Additional kits of the invention comprise a eukaryotic cell capable of transiently inducing a decreased level of non-homologous recombination activity; and, one or more isolated polynucleotide comprising a homologous recombination cassette, as described above; or a polynucleotide or polypeptide which when expressed or introduced in said eukaryotic cell further increases homologous recombination activity in said eukaryotic cell. In specific embodiments, the polynucleotide which when expressed in said eukaryotic cell further increases homologous recombination activity, is designed to allow for a transient modulation of homologous recombination activity. comprises All of the relevant compositions discussed above, can be included in such a kit.

Any kit of the invention can further comprise one or more sets of instructions. The set of instructions can comprise instructions for reducing the level of non-homologous recombination activity in a desired host cell, methods of preparing RNAi molecules, methods of introducing a polynucleotide of interest or a desired target site into the homologous recombination cassette, and/or methods for the introduction of the silencing element and/or the homologous recombination cassette into the host cell.

The kits of the invention can further comprise nucleic acids (primers, vectors, etc.), enzymes (ligase, Clonase™, topoisomerase, etc.) or buffers useful for cloning into the homologous recombination cassette either the regions of homology to the target site and/or the polynucleotide of interest.

Liquid components of kits are stored in containers, which are typically resealable. A preferred container is an Eppendorf tube, particularly a 1.5 ml Eppendorf tube. A variety of caps may be used with the liquid container. Generally preferred are tubes with screw caps having an ethylene propylene O-ring for a positive leak-proof seal. A preferred cap uniformly compresses the O-ring on the beveled seat of the tube edge. Preferably, the containers and caps may be autoclaved and used over a wide range of temperatures (e.g., +120 Cto-200 C) including use with liquid nitrogen. Other containers can be used.

IIX. Sequence Identity

As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

IX. Variants and Fragments

By “fragment” is intended a portion of the polynucleotide or a portion of the amino acid sequence and hence protein encoded thereby. Fragments of a polynucleotide may encode protein fragments that retain the biological activity of the native protein. Alternatively, fragments of a polynucleotide that are useful as a silencing element do not need to encode fragment proteins that retain biological activity. Thus, fragments of a nucleotide sequence may range from at least about 10, about 15, 20 nucleotides, about 50 nucleotides, about 75 nucleotides, about 100 nucleotides, 200 nucleotides, 300 nucleotides, 400 nucleotides, 500 nucleotides, 600 nucleotides, 700 nucleotides and up to the full-length polynucleotide employed in the invention. Methods to assay for the activity of a desired silencing element or for the overepxressed polynucleotide and/or polypeptide are described elsewhere herein.

“Variants” is intended to mean substantially similar sequences. For polynucleotides, a variant comprises a deletion and/or addition of one or more nucleotides at one or more internal sites within the native polynucleotide and/or a substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of one of the polypeptides employed in the invention. Variant polynucleotides also include synthetically derived polynucleotide, such as those generated, for example, by using site-directed mutagenesis, but continue to retain the desired activity. Generally, variants of a particular polynucleotide of the invention having the desired activity will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Variants of a particular polynucleotide of the invention (i.e., the reference polynucleotide) can also be evaluated by comparison of the percent sequence identity between the polypeptide encoded by a variant polynucleotide and the polypeptide encoded by the reference polynucleotide. Percent sequence identity between any two polypeptides can be calculated using sequence alignment programs and parameters described elsewhere herein. Where any given pair of polynucleotides employed in the invention is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

“Variant” protein is intended to mean a protein derived from the native protein by deletion or addition of one or more amino acids at one or more internal sites in the native protein and/or substitution of one or more amino acids at one or more sites in the native protein. Variant proteins encompassed by the present invention are biologically active, that is they continue to possess the desired biological activity of the native protein, as discussed elsewhere herein. Such variants may result from, for example, genetic polymorphism or from human manipulation. Biologically active variants of a native protein will have at least about 40%, 45%, 50%, 55%, 60%, 65%; 70%, 75%, 80%, 85%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein of the invention may differ from that protein by as few as 1-15 amino acid residues, as few as 1-10, such as 6-10, as few as 5, as few as 4, 3, 2, or even 1 amino acid residue.

The present invention will be better understood with reference to the following non-limiting examples.

### Experimental

### EXAMPLE 1

Non-homologous end joining (NHEJ) is the major DNA double-strand break (DSB) repair pathway in mammalian cells and is likely responsible for the non-homologous integration of transgenes. In higher eukaryotes, this pathway predominates over the homologous recombination (HR) pathway and therefore may account for the low level of HR events that occur in mammalian cells. In an attempt to manipulate exogenous DNA integration, we evaluated the effects of the transient RNAi-induced down-regulation of key components of the NHEJ pathway in human HCT116 cells. Treatment with siRNA targeting Ku70 and Xrcc4 reduced corresponding protein levels by 80-90% 48 h after transfection, with a return to normal levels by 96 h. Additionally, down-regulation of Ku 70 and Xrcc4 resulted in a concomitant down regulation of both Ku70 and Ku86 proteins. Biological consequences of transient RNAi-mediated depletion of Ku70 and Xrcc4 included sensitization to γ radiation, decreased cell survival and a significant decrease in the expression of a linear GFP reporter gene, indicating inhibition of non-homologous transgene integration into the genome. The results implicate NHEJ proteins in DNA integration events in human cells, highlighting the possibility of successful means to manipulate the NHEJ pathway by RNAi for use in gene targeting.

Introduction

The integration of exogenous DNA into chromosomes involves DNA double-strand break (DSB) repair components, with foreign DNA being integrated into the genome during or via the repair of a break (Orr-Weaver and Szostak (1983) Mol. Cell. Biol. 3:747-749). The two major DSB repair pathways in eukaryotes are the homologous recombination (HR) and non-homologous end joining (NHEJ) pathways (Van Gent et al. (2001) Nat. Rev. Genet. 2:196-206; Jackson, S. P. (2002) Carcinogenesis 23:687-696). The relative contributions of these pathways in DSB repair depend on the organism, developmental stage, cell cycle and, to a certain extent, the structure of the break. In organisms with a compact genome, including bacteria and lower eukaryotes, such as Saccharomyces cerevisiae, DSB are primarily repaired via HR, the NHEJ pathway being utilized only if the HR mechanism is impaired (Siede et al. (1996) Genetics 142:91-10). Higher eukaryotes are capable of utilizing both pathways for DSB repair; however, NHEJ appears to predominate (Chu, G. (1997) J. Biol. Chem. 272:24097-24100). The integration of foreign DNA, even with a large amount of sequence homology, also seems to occur preferentially via the NHEJ pathway (Sedivy and Dutriaux (1999) Trends Genet. 15:88-90). Such pathway preference by different organisms raises interesting questions regarding control of pathway regulation and may offer insight on approaches to minimize the impact of NHEJ and increase HR for the insertion of transgenes.

The HR pathway requires extensive DNA homology and the outcome is accurate and conservative, precisely restoring the DNA molecule by using a homologous DNA sequence as a template. In contrast, the NHEJ pathway will join two broken DNA ends with little or no sequence homology (Paques and Haber (1999) Microbiol. Mol. Biol. Rev. 63:349-404). This process requires several factors that will sequentially recognize and bind the broken ends, catalyze the synapses, and then process and reseal the break (Lees-Miller and Meek (2003) Biochimie 11:1161-1173; Lieber et al. (2003) Nat. Rev. Mol. Cell. Biol. 4:712-720). The known proteins involved in the NHEJ pathway consist of Ku70/Ku86, the complex DNA-dependent protein kinase catalytic subunit (DNA-PKcs), the Artemis nuclease, DNA ligase IV, and its co-factor Xrcc4 (Lieber et al. (2003) Nat. Rev. Mol. Cell. Biol. 4:712-720).

This phylogenetically conserved group of proteins acts in a coordinated fashion to repair DNA breaks. The Ku heterodimer, comprised of Ku70 and Ku86 subunits, is the first component to bind the ends of a DSB (Critchlow and Jackson (1998) Trends Biochem. Sci. 23:394-398). Ku is an abundant cellular and structure-specific DNA binding protein and requires a free DNA end for binding to occur (Mimori and Hardin (1986) J. Biol. Chem. 279:10375-10379; Yoo et al. (1999) “Photocross-linking of an oriented DNA repair complex. Ku bound at a single DNA end,” J. Biol. Chem. 274:20034-20039). The crystallization of the Ku heterodimer revealed that the protein forms a ring tethering the DNA ends (Walker et al. (2001) Nature 412:607-614). Biochemical data suggest that a cascade of events is initiated upon DNA binding by the Ku complex that leads to the joining of two DNA ends. In this sequence of events, Ku bound to DNA ends attracts DNA-PKcs to form the DNA-PK holoenzyme, which becomes an active kinase. The DNA-PKcs complex, along with other auxiliary factors, stimulate end processing for subsequent ligation (Moshous et al. (2001) Cell 105:177-18) by Xrcc4 and DNA ligase IV (Critchlow and Jackson (1998) Trends Biochem. Sci. 23:394-398). The latter is a dedicated enzyme that appears to be involved only in NHEJ and which requires physical interaction with the Xrcc4 protein to function (Grawunder et al. (1998) Mol. Cell. 2:477-484).

Rodent cells containing permanent mutations in NHEJ proteins show premature senescence and severe sensitivity to radiation as a consequence of their inability to repair DNA DSBs (Ferguson et al. (2000) Proc. Natl. Acad. Sci. USA 97:6630-6633). Mutations in DNA ligase IV and Xrcc4 are embryonically lethal in mice (Barnes et al. (1998) Curr. Biol. 8:1395-1398; Frank et al. (1998) Nature 396:173-177); however, human cells can tolerate DNA ligase IV mutations (Grawunder et al. (1998) Mol. Cell. 2:477-484; So et al. (2004) J. Biol. Chem. 279:55433-5544). In addition, Ku86 is an essential gene in human somatic cells, while mouse knockout models are viable (Li et al. (2002) Proc. Natl. Acad. Sci. USA 99:832-837). In mice, mutations in DNA-PKcs and Artemis result in radiosensitivity and the inability to resolve V(D)J recombination intermediates (Taccioli et al. (1998) Immunity 9:355-66; Rooney et al. (2002) Mol. Cell. 10:1379-1390). The down-regulation or knockout of either Ku70 or Ku86 results in the reciprocal down regulation of the other subunit (Nussenzweig et al. (1996) Nature 382:551-555; Min et al. (2004) Proc. Natl. Acad. Sci. USA 101:8906-8911). However, the relationship between the regulation of core NHEJ proteins is still puzzling and generally unknown in other cell types.

Although the dominant DSB repair pathway in the cell appears to direct the mode and frequency of exogenous DNA integration in a host genome, the molecular mechanisms of foreign DNA integration in mammalian cells is still not resolved and requires further investigation. By transient down-regulation of key NHEJ components in cell culture using RNA interference (RNAi), loss-of-function phenotypes can be assessed irrespective of an essential role for any given protein. RNAi prevents protein production by a post-transcriptional process and does not require manipulation of the genome. In contrast to mutant cell lines, with RNAi, transient inhibition of protein expression is possible, thereby allowing the analysis of protein function in stable cell lines or organisms during a window of induced protein depletion. The objectives of this study were designed to assess the biological effects of transient Ku70 and Xrcc4 loss-of-function and the subsequent consequences on the integration of exogenous DNA into the genome. Here we demonstrated that the frequency of random DNA integration can be significantly reduced by the depletion of NHEJ proteins, thereby highlighting potential applications in gene targeting and gene and cancer therapy. In addition, our results indicated that levels of Ku70 and Ku86 were almost undetectable in Xrcc4 down-regulated cells, suggesting a possible new role for Xrcc4 in the coordination of DNA end-processing by Ku complex proteins.

Materials and Methods

Small Interfering RNA (siRNA)

Segments of the human Ku70 (167 bp in exon 5 from sequence position 407 to 574; and 249 bp in exon 13 from nucleotides 1764 to 1989; Gene Bank accession no. NM001469) and Xrcc4 (172 bp in exon 2 from sequence position 317 to 489; and 108 bp in exon 6 from nucleotides 858 to 1067; Gene Bank accession no. NM003401) genes were chosen to generate double-stranded RNA using the BLOCK-iT™ T7-TOPO® Linker (Invitrogen) for subsequent “Dicing” to create pools of siRNA using BLOCK-iT™ Dicer RNAi kit (Invitrogen) and stored at −80° C. The siRNA generated from both fragments for each gene were pooled.

Cell Culture

Human colon cancer cells HCT116 (ATCC, Manassas, Va., USA) were cultured as monolayers in McCoy's 5A medium supplemented with 10% fetal bovine serum (Sigma Chemical) and antibiotic-antimycotic solution (penicillin-streptomycin-amphotericin b, Invitrogen). Cells were maintained in a humidified atmosphere at 37° C. in 5% CO₂. Prior to siRNA transfection, cells were grown in antibiotic-antimycotic-free media.

siRNA Transfections

HCT116 cells were plated at 30% confluence in 24- or 6-well culture plates. The following day, siRNA fragments (400 nM for Ku70, 200 nM for Xrcc4 and 200 nM non-specific siRNA as negative control) and Lipofectamine 2000 (1%, v/v, Invitrogen) were prepared in OptiMEM I (Invitrogen), according to the manufacturer's instructions, and added to cells (100 μL/well for 24-well plates or 500 μL/well for 6-well plates). After 4-6 h, 5 volumes of culture medium were added to the siRNA-containing medium, for a final siRNA concentration of 80 nM for Ku70 or 40 nM for Xrcc4 and negative control, respectively. Each transfection was done in duplicate or triplicate, and controls consisted of mock-transfected cells (sham, Lipofectamine 2000 only) and a non-specific siRNA (negative control, Medium GC Stealth™, Invitrogen).

Immunoblots

The knockdown of specific targeted gene products was monitored by Western blot. HCT116 cells were trypsinized, washed with PBS and resuspended in RIPA buffer (Santa Cruz Biotechnology). Total protein content of all extracts was quantified using the Bradford assay (Bio-Rad). Protein extracts (5 μg of total protein) were boiled for 5 min and loaded on a 10% SDS-PAGE gel for separation. After migration, the proteins were transferred to nylon membranes (Hybond P, Amersham) and probed with primary monoclonal antibodies to Ku70 (MS-329), Ku86 (MS-285, Neomarkers); polyclonal goat anti-Xrcc4 (sc-8285, Santa Cruz Biotechnology), and a monoclonal GAPDH (ab9484, Abcam) as a loading control. Blots were then incubated with HRP-conjugated secondary antibodies (Santa Cruz Biotechnology) and visualized using the ECL detection kit (Amersham). Protein abundance was estimated relative to GAPDH levels using Alphaease FC software (Alphainnotech Corporation).

γ-Irradiation and Cell Survival

A ¹³⁷Cs source (Institute of Toxicology and Environmental Health, University of California, Davis) was used to irradiate cells for a total dose of 8 Gy based on the ability of this dose to inflict DSBs. Cells were irradiated as monolayers in 6-well plates 48 h after si-RNA transfection. After irradiation, cells were trypsinized and divided for cell cycle and survival assays. For cell cycle analysis, approximately 100,000 cells/plate were incubated for 24 h before trypsinization and evaluation of cell cycle distribution by flow cytometry. To quantify cell survival after exposure to radiation, cells were plated in duplicate at 10,000 cells/100-mm plate, and after an additional 10 days of incubation, colonies were fixed and stained with crystal violet for counting. Four independent experiments were preformed in triplicate.

GFP Expression Assay

The pEGFP-NI (Clontech) vector was linearized by digestion with AflII resulting in a 4.8 Kb DNA fragment with GFP driven by the CMV promoter. The linearized plasmid (0.5 μg/well for 24-well plate) was transfected into HCT116 cells at 48 or 96 h after the siRNA transfection by the same delivery method described for siRNA. GFP-expressing cells were measured by flow cytometry 72 h after pEGFP-N1 transfection. A minimum of 4 independent experiments conducted in duplicate for each siRNA treatment and each time point (48 or 96 h) were preformed.

Flow Cytometry

Cellular DNA content and GFP-expressing cells were analyzed by fluorescence-activated cell sorting using a FACScan machine with Cell Quest software (Becton Dickinson). The distribution of cells at different stages of the cell cycle was measured by standard methods using propidium iodide staining of DNA (Taylor and Milthorpe (1980) J. Histochem. Cytochem. 28:1224-1232). Briefly, samples were resuspended in 0.01% propidium iodide (ICN Biomedicals) and 2 mg/mL RNase A (Calbiochem) and incubated at RT for 30 min in the dark. Analysis of GFP expression was done with freshly trypsinized and paraformaldehyde-fixed (1%, w/v) cells. The proportion of GFP-positive cells was scored in dot-plots of GFP fluorescence versus FL3 autofluorescence, using mock-transfected (sham) cells to define the negative area. DNA integration efficiency was then estimated as the percentage of GFP-positive cells.

Statistical Analysis

Comparisons were made by constructing 95% confidence intervals on data regarding protein analysis by densitometry, flow cytometry/cell survival analysis, and GFP expression. Cell cycle analysis was performed by Cell Quest ModFitLT software, v.2.0, and data were analyzed by proportions with stratified sampling. The nature of the relationships between variables obtained in this study was carried out by a Pearson's correlation and/or a simple linear regression tests (Minitab Inc.).

Results

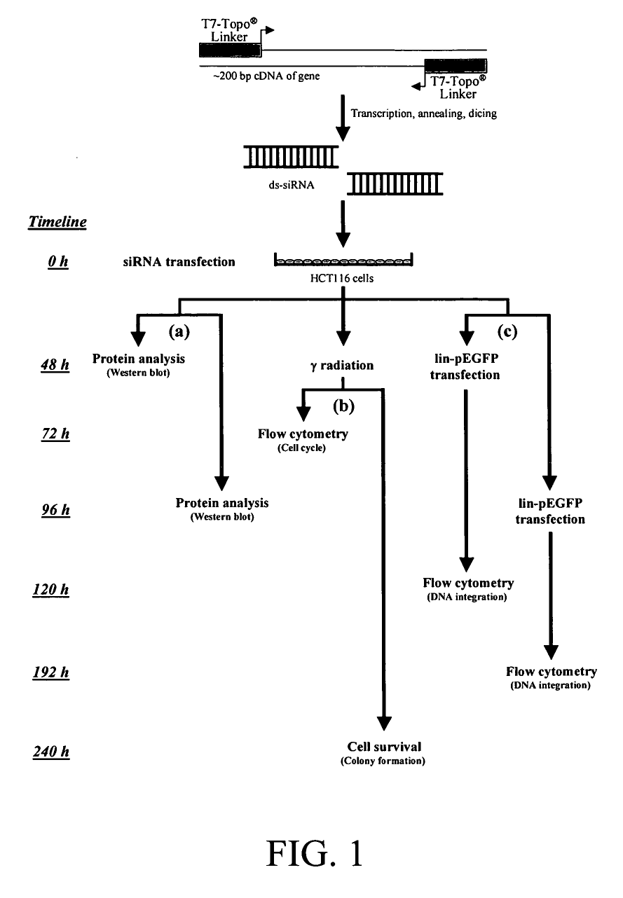

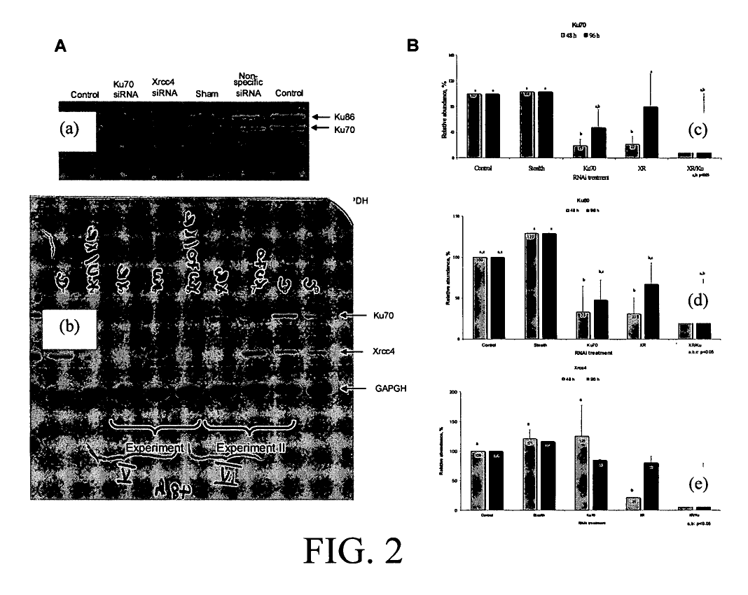

The transient down-regulation of upstream and downstream components of the NHEJ pathway was capable of effecting cellular functions. The RNAi-induced depletion of the Ku70 and/or Xrcc4 proteins in HCT116 cells influenced the ability of the cells to survive DNA damage induced by γ irradiation and integrate DNA randomly into the genome. A diagram of the experimental design, including a timeline, is shown in FIG. 1. The optimal conditions for RNAi depletion of Ku70 and Xrcc4 proteins were determined by varying siRNA concentration (10 nM to 1.5 μM) and the amount of Lipofectamine 2000 (0.5 μl to 1.5 μl), and by monitoring protein levels by western blotting 24, 48, 72 and 96 h after siRNA treatment (data not shown). Optimal inhibition of Ku70 and Xrcc4 proteins was observed 48 h after siRNA treatment using 400 nM of ds-siRNA for Ku70 and 200 nM for Xrcc4. These conditions were adopted as a standard protocol for Ku70 and Xrcc4 RNAi treatments. Mock transfected cells (sham, Lipofectamine 2000 only) and cells transfected with a non-specific siRNA negative control (200 nM) showed no detectable changes in the levels of the specific proteins measured in this study (FIG. 2).

Ku70 and Xrcc4 Knockdown

Treatment of HCT116 cells with siRNA fragments targeting Ku70 and Xrcc4 transcripts significantly induced the down-regulation of the respective proteins to approximately 15% of normal levels at 48 h post-transfection (P<0.05), with a complete or partial return to control levels by 96 h (FIGS. 2C, D, E). RNAi targeting Ku70 induced down-regulation of both Ku70 and Ku86 proteins 48 h after the siRNA transfection (FIGS. 2A, B). Ku70 inhibition had no influence on Xrcc4 protein levels at 48 h (FIG. 2B).

However, siRNA targeting Xrcc4 not only induced the down-regulation of Xrcc4, but also Ku70 and Ku86 (P<0.05; FIGS. 2C, D, E). Combined transfection of cells with siRNA targeting both Ku70 and Xrcc4 (each at half the doses used for individual siRNA transfections) also induced down-regulations in Ku70 and Ku86 to approximately 20% of normal levels at 48 h (P<0.05), with a return to control levels at 96 h post-transfection (FIGS. 2C, D). However, the combined siRNA treatment, although significantly lower than controls (P<0.05), did not decrease the Xrcc4 levels to the same extent observed when treated with twice the dose (40 vs. 21%, respectively), as shown in FIG. 2E. Treatment of cells with no siRNA (sham transfection, not shown) and with 200 nM of a non-specific, negative control siRNA (FIG. 2A-E) did not affect the relative abundance of Ku70, Ku86 or Xrcc4 proteins at either 48 or 96 h post-transfection, which were comparable with non-transfected cells. Levels of Rad51 and Chk1 proteins measured by Western blotting were not changed by any of the siRNA treatments (data not shown).

Effects of Ku70 and Xrcc4 Depletion on Cell Cycle and Cell Survival Following Irradiation

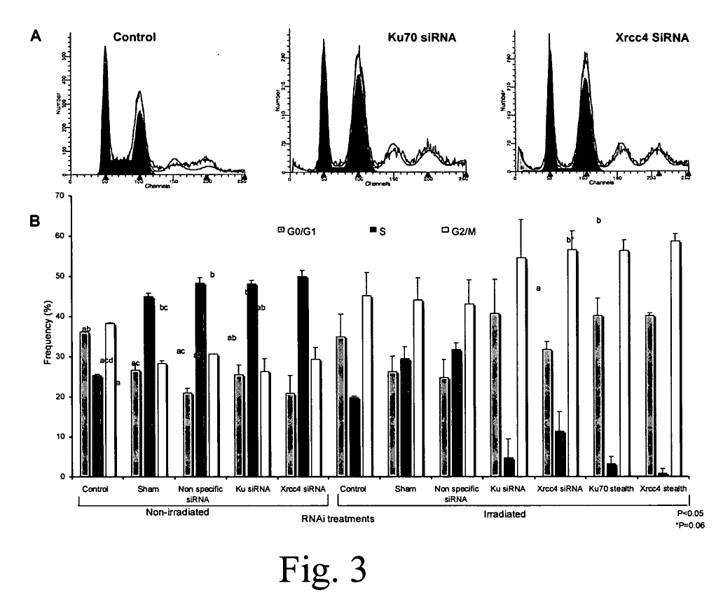

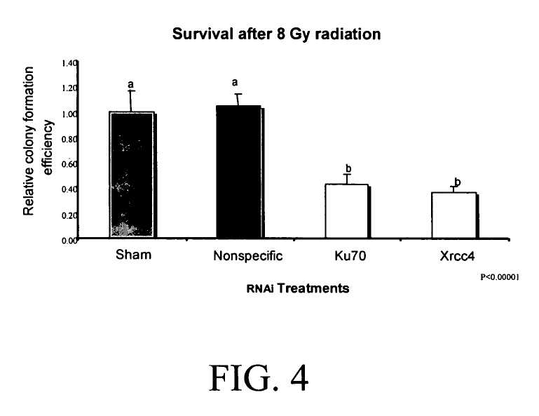

To determine if the transient depletion of Ku70 and Xrcc4 to approximately 15% of normal levels was sufficient to have a biological effect, siRNA-treated cells were analyzed for sensitivity to γ irradiation by evaluating their ability to survive and progress through the cell cycle. Four independent experiments, each assayed in triplicate, demonstrated that depletion of Ku70 or Xrcc4 caused increased sensitivity to γ radiation and changes in the cell cycle (FIG. 3). Ku70 or Xrcc4 siRNA treatments and subsequent exposure to 8 Gy of γ radiation significantly increased the proportion of cells in G2/M phase (P<0.05) and decreased cells in S phase (P<0.05) compared with sham and non-specific siRNA groups and controls (FIGS. 3A, C). Cells treated with Ku70 siRNA also had more cells in G0/G1 than controls (FIGS. 3A, C). In addition, depletion of Ku70 or Xrcc4 by siRNA transfection resulted in a significant decline in cell survival upon exposure to γ radiation (P<0.00001; FIG. 4). The siRNA treatments did not affect the proportion of cells in the various stages of the cell cycle in non-irradiated cells (FIGS. 3A, B). Changes in cell cycle (decrease in G0/G1 and G2/M phases and increase in S phase) were similar between all transfected groups (sham, non-specific, Ku70 and Xrcc4 siRNA-treated cells) but different from non-transfected controls. This was likely an effect due to the transfection (lipofection) treatment per se rather than to the siRNA treatment.

Depletion of Ku 70 and Xrcc4 Resulted in a Decrease in Random DNA Integration

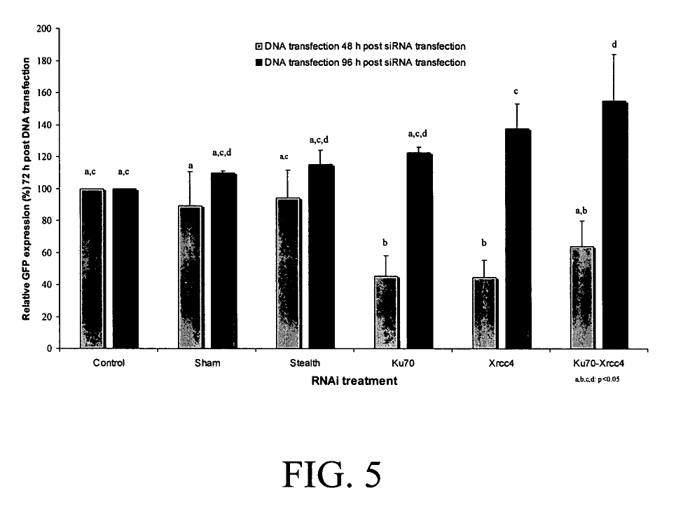

To determine if the transient down regulation of NHEJ components could influence DNA integration, as proof of principle, a linearized GFP expression vector was transfected into HCT116 cells 48 h or 96 h post-siRNA treatment, and the percentage of GFP expressing cells was measured by flow cytometry 72 h after the introduction of the linearized reporter construct. Compared with controls, the down-regulation of Ku70 and/or Xrcc4 in HCT116 cells negatively affected GFP expression when the reporter gene was introduced at 48 h post-siRNA transfection, but not when introduced at 96 h (FIG. 5). The transfection of the linear reporter gene at 96 h post-siRNA transfection resulted in levels of GFP expressing cells similar to controls or at a higher level in the case of Xrcc4/Ku70 combined treated cells. Levels of GFP expression were not affected in sham and non-specific siRNA transfected cells.

Discussion

We have applied RNAi technology to transiently deplete cells of Ku70 and Xrcc4 in order to examine the role of the NHEJ pathway in exogenous DNA integration. Human cancer cells (HCT116) were chosen for this work as they are known to have a high transformation efficiency and are commonly used in recombination studies. In this study, siRNA molecules targeting Ku70 and Xrcc4 successfully resulted in an 80 to 90% reduction in their corresponding protein levels 48 h after transfection which resulted in the cells being sensitive to γ radiation and impaired their ability to integrate a non-homologous reporter gene construct as indicated by decreased reporter gene expression.

In eukaryotic cells, DNA repair by NHEJ is initiated by Ku heterodimer binding to DNA free ends and is completed when the DNA ends are sealed by the Xrcc4/DNA ligase IV complex (Critchlow and Jackson (1998) Trends Biochem. Sci. 23:394-398). Treatment of HCT116 cells with siRNA specific to Ku70 or Xrcc4 mRNA resulted in the maximum transient down-regulation of the respective protein by 48 h post-transfection with a recovery to pre-treatment levels by 96 h post-transfection. Higher levels of Ku70 siRNA were required to achieve this effect than for Xrcc4, most likely due to the fact that Ku70 is a more abundant cellular protein. Depletion of Ku70 also resulted in the concomitant down-regulation of Ku86. These results are in agreement with data collected using mutant rodent cells that showed a parallel depletion of Ku70 and Ku86 (Ferguson et al. (2000) Proc. Natl. Acad. Sci. USA 97:6630-6633; Gu et al. (1997) Immunity 7:653-665) and also from results of RNAi studies in Drosophila (Barnes et al. (1998) Curr. Biol. 8:1395-1398). One study in human cells using an antisense RNA to Ku86 reported no decrease in Ku70 expression (Marangoni et al. (2000) Cancer Gene Ther. 7:339-346). However, knowledge about NHEJ protein stabilization and regulatory mechanism is very limited in human cells.

In this report, we also describe interactions between the co-factor Xrcc4 and the Ku complex. The depletion of Xrcc4 resulted in the coordinated down-regulation of both Ku70 and Ku86 proteins (FIG. 2). This may indicate a regulatory role for Xrcc4 in an earlier step in NHEJ pathway, as other researchers (Koch et al. (2004) EMBO J. 23:3874-3885) proposed that Xrcc4 acts as a NHEJ scaffold, targeting PNK and DNA ligase IV to DSB, and thereby physically linking the enzymatic reactions of DNA end processing and DNA ligation. It is also known that Xrcc4 can bind, stabilize and stimulate DNA ligase IV (Critchlow et al. (1997) Curr. Biol. 7:588-598; Grawunder et al. (1997) Nature 388:492-495; Bryans et al. (1999) Mutat. Res. 433:53-58; Modesti et al. (1999) “DNA binding of Xrcc4 protein is associated with V(D)J recombination but not with stimulation of DNA ligase IV activity,” EMBO J. 18:2008-201; and Chen et al. (2000) J. Biol. Chem. 275:26196-26205). Our results indicated that levels of Xrcc4 protein are either directly or indirectly influencing the amount of Ku proteins in human cells. This reduction in Ku70 and Ku86 protein levels by Xrcc4 depletion may be due to a reduced stability of the Ku complex, perhaps by the failure of the complex to form. Xrcc4 may also have a regulatory role that is influencing Ku70 and Ku86 expression; such possibilities need to be clarified by further research.

To date, only a few mutant human cell lines have been isolated for NHEJ proteins, including one mutant DNA-PKcs cell line and, more recently, a ligase VI null cell line (Allalunis-Turner et al. (1995) Radiat. Res. 144:288-293; Taccioli et al. (1998) Immunity 9:355-66). In addition, there is no description of any human patient harboring a mutation in Ku subunits or for DNA-PK. Ku70 mutant cell lines have not been described, while Ku86 was proven to be an essential gene (Rooney et al. (2002) Mol. Cell 10:1379-139). Studies in human cells using an antisense approach to down regulate Ku70 and Ku86 similarly reported radiosensitive phenotypes, providing strong evidence that the Ku complex is involved in DSB repair in human cells (Marangoni et al. (2000) Cancer Gene Ther. 7:339-346; Jeanson et al. (2002) “Effect of Ku86 depletion on the preintegrative steps of HIV-1 replication in human cells,” Virology 300:100-108; Omori et al. (2002) DNA Repair 1:299-310). Although these reports show radiosensitization with partially reduced levels of Ku, they were inconclusive with respect to a concomitant decrease in the other Ku subunit, which contrasts findings in rodent cells and this study. Here, we show a significant decrease in Ku86 to less than 20% of normal cellular levels concomitant with the reduction of Ku70, as well as a decrease in both Ku70 and Ku86 concomitant with Xrcc4 depletion. The transient nature of this approach to studying NHEJ components, as opposed to most other reports in using mutant cell lines, may also account for the differences seen in interactions among the component proteins.

A phenotype resulting from transient Ku70 and Xrcc4 depletion became apparent after cells were exposed to ionizing radiation, a known source of DNA damage. These results are in agreement with recent data showing that a reduction in human Ku70 protein by RNAi caused an increase in cell sensitization to ionizing radiation (Ayene et al. (2005) Mol. Cancer Ther. 4:529-536). Such findings are of interest since targeting the NHEJ pathway may have valuable therapeutic applications. The reduction in Ku70 and Xrcc4 proteins also caused a stronger G₂ checkpoint response 24 h after the ionizing radiation, which was shown by both the accumulation of cells in G₂ phase and the lack of cells in S phase. Treated cells were likely to be in S or G₁ phases at the time of irradiation, as indicated by the distribution in non-irradiated cells (61 to 73% of cells were in G0/G1 or S phases, FIG. 3), which indicates a G2/M phase accumulation of cells that were early in the cell cycle at the time of irradiation. The higher accumulation in G1 and G2 likely reflected the build-up of non-repaired DNA DSB in cells with reduced levels of NHEJ proteins.

A decrease in the number of GFP expressing cells was evident after the transfection of a linearized reporter construct at the time of maximum down-regulation of Ku 70 and Xrcc4 protein (i.e. 48 h after siRNA treatment, FIG. 5). The decreased GFP expression correlates with the increase in radiosensitivity seen in Ku70 and Xrcc4 siRNA-transfected cells and is likely to be related to a decrease in exogenous DNA integration. This agrees with the results of a previous study showing that plasmid DNA integration was decreased in NHEJ mutant CHO cells (Hamilton and Thacker (1987) Mol. Cell Biol. 7:1409-1414; Jeggo and Smith-Ravin (1989) Mutat. Res. 218:75-86). The spatial and temporal coordination of DNA integration events is almost certainly directly and/or indirectly mediated by DNA-protein-protein interactions. Still, relatively little is known regarding the mechanisms of NHEJ-mediated exogenous DNA integration. Lower levels of DNA integration in RNAi-induced NHEJ-depleted cells could be a consequence of fewer protein-DNA complexes available to participate in DSB repair, and thus transgene integration. Alternatively, shortened exogenous DNA lifespans due to increased degradation of unprotected DNA could decrease integration (Liang and Jasin (1996) J. Biol. Chem. 271:14405-14411), since DNA end protection from nucleases may also decrease with depletion of key NHEJ components (e.g., Ku70, Ku86). Recently, a chromatin remodeling protein named Metnase (a putative NHEJ protein), has also been implicated in exogenous DNA integration and DNA repair in human 293 cells (Lee et al. (2005) Proc. Natl. Acad. Sci. USA 102:18075-18080). By promoting histone methylation, Metnase indirectly appears to facilitate DNA end joining by opening chromatin, creating novel opportunities for the manipulation of the recombination processes in eukaryotic cells. Nevertheless, the mechanisms and associations between known and putative NHEJ components and DNA integration require further investigation.

Data from several laboratories corroborate that components of the NHEJ pathway mediate retroviral DNA integration (Omori et al. (2002) DNA Repair 1:299-310; Li et al. (2001) “Role of the Nonhomologous DNA end joining pathway in the early steps of retroviral infection,” EMBO J. 20:3272-3281; and Daniel et al. (2004) J. Virol. 78:8573-8581). Furthermore, Ku86 is required for restriction enzyme-mediated integration (REMI) in mammalian cells (Manivasakam et al. (2001) Nucleic Acids Res. 29:4826-4833). We show here that DNA integration is reduced with depletion of the Ku70 or Xrcc4 in human cells, strengthening the implication of NHEJ proteins in the process of DNA integration in human cells. However, since the depletion of Xrcc4 protein was associated with a parallel decrease in the levels of Ku70 and Ku86, targeting Xrcc4 protein expression by RNAi may be a more efficient approach to down-regulate the NHEJ pathway.

An understanding of the machinery that mediates DNA integration will advance the development of new technologies in genetic engineering. The usually low frequency of HR and high frequency of random integrations are impractical for the application of permanent modifications in the genome of higher eukaryotes. This reality has hindered progress, as insertional mutations are a risk for any gene targeting approach. A reduction in random DNA integration following transfection would be beneficial and advantageous for the screening of targeted insertions when no enrichment protocols are available, as in the case of transgenic animals or human gene therapy. Our results indicate that random DNA integration can be reduced by transient down-regulation of the NHEJ pathway components, which could be favorable for such purposes. In general, the ratio of homologous recombination to non-homologous recombination events oscillates between 1:10⁴ to 1:10⁸, depending on gene locus and cell type (Sedivy and Sharp (1989) Proc. Natl. Acad. Sci. USA 86:227-231; Porteus and Baltimore (2003) Science 300:762). These very rare legitimate recombination events require tedious DNA screening for the identification of colonies containing desired mutations. Thus, down-regulation of the NHEJ pathway may be one way to enrich for homologous integration of exogenous DNA.

### EXAMPLE 2

### Transiently Down-Regulating Key Components (Ku70, DNA Ligase IV and Xrcc4) of the NHEJ Pathway by RNAi and Measure Gene Insertion

Reagents and optimizing conditions for the RNAi-induced down regulation of NHEJ pathway components in human HCT116 cells as well as for other cell types (for use in Example 5) were preformed. The goals of this example were to identify appropriate siRNA knock-down reagents and the optimum dose and time required to achieve a transient 70-90% down-regulation of the target protein. The targets proposed for use were Ku70, DNA ligase IV and Xrcc4 due to their significance to the NHEJ pathway. Two types of RNAi molecules were evaluated for each target; diced pools of siRNA and Stealth™ siRNA molecules. Stealth™ siRNA molecules, which have proprietary modifications that increase their stability and specificity towards a specific target, were developed. Each siRNA was transfected individually or in combination into HCT116 cells at various concentrations ranging from 10 nM to 1.5 μM. The down-regulation of each targeted protein was monitored over a time course of 0, 24, 36, 48, 72 and 96 hours post-transfection by quantitative western blotting using GAPDH as a loading control. Quantitative real time RT-PCR was also conducted to monitor mRNA levels for each targeted gene.

Both the diced and Stealth™-generated siRNAs were capable of inducing the required transient down-regulation of the targeted protein. The use of diced siRNA at 400 nM (Ku70) or 200 nM (Xrcc4) resulted in an 85% down-regulation of the respective target protein 48 hours after transfection, with recovery to pre-transfection levels by 96 hours post-transfection. Quantitative real time RT-PCR results demonstrated that mRNA levels for the targeted gene were also lowered in accordance with siRNA treatment, indicating that the siRNAs were gene-specific. The same level of down-regulation was found with the use of the Stealth™ siRNA, however, the amount required to achieve the effect was lower (40 nM), thus making them more efficient and cost-effective to use. The co-transfection of siRNA for both Ku70 and Xrcc4 did not dramatically improve on the results obtained with individual component transfections. As a maximum of 400 nM of siRNA could only be used per transfection in order not to have adverse affects on the cells, each siRNA was only present at half the amount as it was when transfected individually. Therefore, with these optimized conditions for a single transfection with siRNA for Ku70 or Xrcc4, we were able to achieve a 70-90% transient down-regulation of these key NHEJ proteins.

In order to determine if transient Ku70 and Xrcc4 depletion would have a biological effect, the sensitivity of RNAi-treated cells to γ radiation, a known source of DNA damage was tested. We hypothesized that if the NHEJ pathway was impaired, a radiation-sensitive phenotype would result. Radiation sensitivity was analyzed by exposing siRNA-treated cells and appropriate controls to 8 Gy γ radiation 48 hours post siRNA transfection and then evaluating their ability to survive and progress through the cell cycle. Ku70 or Xrcc4 siRNA treatment and subsequent exposure to 8 Gy of γ radiation induced a stronger G₂ checkpoint response (significantly more cells in G2/M phase and significantly fewer in S phase), indicating a build-up of non-repaired DNA double strand breaks in cells with reduced levels of NHEJ proteins. In addition, there was a significant decline in the survival of RNAi-treated cells after exposure to radiation. Therefore, the transient depletion of Ku70 and Xrcc4 to approximately 15% of normal levels caused the predicted phenotype of increased sensitivity to γ radiation by impairing the ability of the cells to repair DNA damage in the form of double strand breaks.

We next determined if the transient down regulation of NHEJ components could influence non-homologous DNA integration by using a GFP reporter construct. A linearized GFP expression vector was transfected 48 hours post-siRNA treatment (at the time of maximum down-regulation of Ku70 and Xrcc4), and the percentage of GFP expressing cells was measured by flow cytometry 72 hours after the introduction of the linearized reporter construct. We hypothesized that there would be a decrease in DNA integration in NHEJ-deficient cells due to their reduced ability to repair double strand breaks. We found that after treatment with siRNA for Ku70 or Xrcc4, there was a 50-60% decrease in the number of GFP-expressing cells, indicating decreased DNA integration.

One interesting and novel finding of this work was the relationship between levels of Ku70 and Xrcc4 in the cell. The RNAi targeting of Ku70 induced down-regulation of both Ku70 and Ku86 proteins 48 hours after the siRNA transfection. This phenomenon has been reported in the literature and is not unexpected as Ku70 and Ku86 act as a heterodimer. Ku70 inhibition had no influence on Xrcc4 protein levels, however, siRNA targeting Xrcc4 not only induced the down-regulation of Xrcc4, but also the concomitant down-regulation of Ku70 and Ku86. This finding is novel in the literature and may indicate an earlier role for Xrcc4 in the NHEJ pathway.

### EXAMPLE 3

### Evaluating the Ability of Selected Recombinase Proteins (RecA, hRAD51 and hRAD54) to Increase the Frequency of Targeted Recombinants

This example is directed at increasing the efficiency of HR in somatic cells by coating a targeting vector with recombinase proteins prior to transfection, thus supplying proteins to promote HR. We proposed to determine the number of random and homologous recombinants generated using the replacement type gene targeting vector HPRT/hyg. As HPRT is an X-linked gene, a single HR event results in a loss of function at the locus in our XY HCT116 cells and resistance to 6-TG, while random integrants will be resistant to hygromycin only. The first recombinase protein chosen for study was hRad 51. The linear 12 kb HPRT DNA integration vector was initially treated with T7 exonuclease to create ˜500 bp single stranded DNA ends (the amount of T7 exonuclease and the incubation time have to be optimized for each tube of enzyme and with each vector to generate ˜500 base single stranded ends). Subsequently, 10 ul of hRad51(97 uM) is incubated with 2-5 ug of the treated vector at 37 deg. C. for 10 min in 25 mM Tris-acetate (pH 7.5), 1 mM ATP, 100 ug/ml BSA, 1 mM DTT, 20 mM KCl. The resulting DNA/hRad51 complexes were transfer to Optimem/Lipofectamine medium for immediate transfection. See, for example, Kiianitsa et al. (2006) Proc. Natl. Acad. Sci. USA 103(26):9767-72, herein incorporated by reference.

The HPRT/hyg targeting vector was transfected into HCT116 cells in the form of naked DNA and DNA coated with hRad51. The introduction of coated DNA into cells using lipofectamine presented a challenge as transfection efficiency was very low. However, doubling the lipfectamine used allowed for the introduction of coated DNA at acceptable rates. It was found that the use of hRad51-coated DNA resulted in a 4-fold decrease in random integration and an approximate 2-fold increase in the number of homologous recombinants.

### EXAMPLE 4

### Determining the Efficiency of Gene Targeting after Transient Down-Regulation Of the NHEJ Pathway with the Use of Exogenous Hr Enzymes

The premise of these experiments is based on the notion that if the predominant pathway for DNA integration (NHEJ) was transiently inactivated, we could shift exogenous DNA integration to the HR pathway and thereby increase the efficiency of HR in living cells. The RNAi work described above for example 2 indicates that the integration of DNA with no homology to endogenous DNA sequences is decreased when the NHEJ pathway is disrupted. Example 4 was designed to determine gene targeting efficiency in cells that have undergone RNAi treatment to down-regulate NHEJ pathway components. Here, the HPRT/hyg targeting vector was transfected into HCT116 cells 48 hours after siRNA treatment with and without hRad51 coating. Based on the targeting vector used, the number of random integrants was determined by counting hygromycin resistant colonies and the number of homologous recombinants by resistance to 6-TG.

Random DNA integration in cells transiently depleted of Ku70 or Xrcc4 was reduced by 65% when naked targeting vector was used. These results are in accordance with GFP expression experiment described for example 2 where less GFP expression was observed in siRNA-treated cells. Therefore, we can conclude that DNA integration is decreased when the NHEJ pathway is impaired. In addition, the absolute gene targeting efficiency in NHEJ down-regulated cells was increased 3-fold. We speculate this may be due to decreased competition among NHEJ and HR proteins for the DNA ends of the introduced targeting vector. We observed the same levels of random and homologous DNA integration regardless of the siRNA used (Ku70 or Xrcc4). Combined, the decrease in random DNA integration and the increase in HR resulted in a 12-fold stimulation of gene targeting efficiency. This represents a vast improvement as the number of colonies required for screening to identify a homologous recombinant is greatly reduced.

### EXAMPLE 5

### Investigating the Role of Cell Cycle and the Use of Other Cell Types on the Efficiency of Gene Targeting after Transient Down-Regulation of the NHEJ Pathway and Use of Exogenous HR Enzymes

Example 5 was designed to translate the results from example 2-4 to other cell types in order to evaluate the applicability of our approach to increasing gene targeting efficiency in addition to investigating the role of cell cycle on HR. We proposed to carry out the same work in mouse embryonic stem (ES) cells and mouse primary fibroblast cell cultures. These cell types were chosen based on their importance as tools for genetic engineering. We have also included the study of pig germ cells (PGCs) and pig primary fibroblasts as the pig is an important and more relevant model animal for human health than is the mouse. 10 potential Stealth™ molecules (5 targeting Ku70 and 5 targeting Xrcc4) for both the mouse and pig have been produced. The

TABLE 1
Sequence for molecules targeting mouse Ku70 (SEQ ID NO:3)

| | | | |
|---|---|---|---|
| Start | Sense RNA Sequence 5′-3′ | Antisense RNA Sequence 5′-3′ | Region |

| | | | |
|---|---|---|---|
| 1037 | CAGUGAGACUCUAUCGGGAAACAAA | UUUGUUUCCCGAUAGAGUCUCACUG | ORF |
| 944 | GAGUUCUGUCCAGGUUAAAGUUUAA | UUAAACUUUAACCUGGACAGAACUC | ORF |
| 589 | GGCCAUGGGUCUGACUACUCUUUGA | UCAAAGAGUAGUCAGACCCAUGGCC | ORF |
| 943 | CGAGUUCUGUCCAGGUUAAAGUUUA | UAAACUUUAACCUGGACAGAACUCG | ORF |
| 526 | CGAGUGCUAGAGCUCGACCAGUUUA | UAAACUGGUCGAGCUCUAGCACUCG | ORF |

TABLE 2

Sequences for molecules targeting mouse Xrcc4
(SEQ ID NO:9)

| | | | |
|---|---|---|---|
| Start | Sense RNA Sequence 5′-3′ | Antisense RNA Sequence 5′-3′ | Region |

| | | |
|---|---|---|
| 15 | AAGCAGAAUCUAUCUUGCUUCUGAA | UUCAGAAGCAAGAUAGAUUCUGCUU |
| 566 | CAAAGAUCCGGAGCUUGCAUAAAUU | AAUUUAUGCAAGCUCCGGAUCUUUG |
| 117 | CCAUUCAGCCUGGACUGCAACAGUU | AACUGUUGCAGUCCAGGCUGAAUGG |
| 76 | AGAACAAUAGGAUCCGGCUUUGUUA | UAACAAAGCCGGAUCCUAUUGUUCU |
| 356 | CAGCUGAAGUCAUAAGAGAUCUCAU | AUGAGAUCUCUUAUGACUUCAGCUG |

TABLE 3
Sequences for molecules targeting Pig Xrcc4 (SEQ ID NO:11)

| | | | |
|---|---|---|---|
| Start | Sense RNA Sequence 5′-3′ | Antisense RNA Sequence 5′-3′ | Region |

| | | |
|---|---|---|
| 356 | CAGCUGAGGUCAUUAGAGAACUUAU | AUAAGUUCUCUAAUGACCUCAGCUG |
| 108 | UACUGAUGGGCAGUCAGCAUGGAUU | AAUCCAUGCUGACUGCCCAUCAGUA |
| 977 | CAGGAGACCUCUUUGAUGAGAUUUA | UAAAUCUCAUCAAAGAGGUCUCCUG |
| 287 | CCUUUGAGAAGAACUUGAAAGAUGU | ACAUCUUUCAAGUUCUUCUCAAAGG |
| 536 | GAUUUAUUCUGGUGCUGAAUGAGAA | UUCUCAUUCAGCACCAGAAUAAAUC |

TABLE 4
Sequence for molecules targeting Pig Ku70 (SEQ ID NO:5)

| | | | |
|---|---|---|---|
| Start | Sense RNA Sequence 5′-3′ | Antisense RNA Sequence 5′-3′ | Region |

| | | |
|---|---|---|
| 1171 | GAGGUCAUGGCAGUGUGCAGAUAUA | UAUAUCUGCACACUGCCAUGACCUC |
| 848 | CGGUGAGGCUUUAUCGGGAAACAAA | UUUGUUUCCCGAUAAAGCCUCACCG |
| 303 | CGUCCUACAGGAAUUGGAUAGUCCA | UGGACUAUCCAAUUCCUGUAGGACG |
| 816 | CAAUUUGGUCCAGAAGGCUUACAAA | UUUGUAAGCCUUCUGGACCAAAUUG |
| 647 | GAGAUAUCAUCAGUGUGGCAGAGGA | UCCUCUGCCACACUGAUGAUAUCUC |

The cDNAs for Ku70 (mouse: NCBI accession no. NM_{—}010247, SEQ ID NO:3; pig; TIGR porcine gene index accession no. TC200078, SEQ ID NO:5); and Xrcc4 (mouse: NCBI accession no. NM_{—}028012, SEQ ID NO:9; pig: TIGR porcine gene index accession no. TC204995, SEQ ID NO:11) from the mouse and the pig have been cloned into individual pSCREEN-iT™ vectors to be used as the target sequence in a lacZ-based assay to determine which of these Stealth™ molecules will be used for work in the pig and mouse cells. Concurrently, targeting vectors analogous to the human HPRT/hyg vector are being assembled to target both pig and mouse HPRT genes to test HR frequencies. Mouse and pig cell lines have been obtained and work is underway to optimize transfection conditions in these cells lines with fluorescent tagged siRNA because transfection efficiency in these primary lines is much lower than in the HCT116 cells. Once effective Stealth™ siRNA molecules have been identified and optimal transfection protocols are determined, work as in example 2 will be carried out to down-regulate Ku70 and Xrcc4 prior to measuring HR frequencies in these mouse and pig cells lines. In addition, we have conducted work in HeLa cells using the optimized conditions for the HCT116 cells and have achieved similar levels of down-regulation of Ku70 and Xrcc4 over the same time course performed in HCT116 cells. This indicates that the approach will be applicable in other cell types.

### EXAMPLE 6

### Gene Targeting is Increased by the Transient Depletion of Ku70 and Xrcc4 in Human Somatic Cells

Abstract

Mammalian cells integrate exogenous DNA predominantly by random insertion regardless of sequence homology. Ideally, gene targeting can knock-out a targeted gene or correct an affected gene by incorporating corrective sequences into a specific site, free from undesirable side effects. However, the efficiency of gene targeting in mammalian cells is low. Given the key role of proteins in the non-homologous end joining (NHEJ) DNA repair pathway, coupled with the observed reduction in integration of foreign DNA in NHEJ-depleted cells, we reasoned that transiently decreasing the levels of NHEJ proteins in cultured cells might favor gene targeting. Ku70 and Xrcc4 are integral components of the NHEJ pathway of cellular DNA double-strand break repair. The transient depletion of Ku70 and Xrcc4 protein from human HCT16 cells resulted in reduced illegitimate integration and elevated gene targeting frequencies at the HPRT locus. These findings strongly support the involvement of NHEJ proteins in foreign DNA integration in vivo, and suggest that the amount or basal activity of the protein can be rate limiting for the process. They also demonstrate that the transient depletion of key components of NHEJ proteins is potentially a viable approach for reducing undesirable non-homologous transgene insertions while increasing the frequency gene targeting in human cells.

Introduction

Exogenous DNA can integrate into chromosomes by two distinct cellular mechanisms: a homologous recombination (HR) dependent process or by illegitimate or random insertion. Exogenous DNA integration is still a process that we cannot predict or control, with illegitimate, or non-homologous, DNA integration being a much more efficient process in mammalian cells then homology-dependent integration. Non-homologous integration is usually a 1000 to 10,000 times more frequent than a targeted, or homologous, events (Smith 2001). Several investigations support the idea that exogenous DNA integration is related to cellular double strand break (DSB) DNA repair mechanisms, e.g. the induction of a DSB by a site-specific endonuclease increases the efficiency of DNA integration (Lin and Waldman, 2001; Dellaire et al., 2002; Jasin 1996). In mammalian cells a DSB can be repaired by homologous recombination (HR) or by the nonhomologous end joining (NHEJ) pathway (Van Gent et al., 2001; Jackson, 2002).

Proteins in the NHEJ pathway include Ku70 and Ku86 (Ku complex), DNA-PKcs, Artemis nuclease, and DNA ligase IV with its co-factor Xrcc4 (Lieber et al., 2003). The ku complex binds to DNA ends and recruits the other factors to finish the repair (Hefferin and Tomkinson, 2005). The role of the NHEJ pathway in exogenous DNA integration is unclear at present. NHEJ repair can be dictated by the stage of the cell cycle and consequently by the availability of the recombination enzymes, and to a certain extent by the structure of the DNA break (Ristic et al., 2003). Furthermore, the effect of a DSB-inducing restriction enzyme on DNA integration was found to be Ku80 dependent (Manivasakam et al., 2001), a key protein in NHEJ pathway. Finally, the lack of NHEJ proteins negatively affect viral DNA integration in rodent cell lines (Li et al., 2001; Daniel et al., 2004).

Homologous Recombination (HR) between transfected and chromosomal DNA could be of great relevance to a broad range of organisms if the efficiency of HR was improved. It is still not practical to think of gene correction in human cells with such low levels of spontaneous gene targeting. Increases in the absolute targeting frequencies have been obtained by maximizing the length and homology of the targeting vector with the target locus (Jasin et al., 1985; Deng and Capecchi 1992; Scheerer and Adair, 1994) or by engineering double-strand breaks (DSBs) in the target locus (Smith et al., 1995). Other approaches included the use of short fragments of DNA (Kunzelmann et al., 1996) and adeno-associated virus vectors (Russel and Hirata 1998). Several research groups are engaged in attempts to increase the efficiency of gene targeting by the overexpression of proteins involved in DSB repair via the HR pathway (Kim et al., 2001; Yanez and Porter 2002; Johnson et al., 1996; Di Primo et al., 2005). The elucidation of the enzymatic mechanism of DNA integration in general would have a great importance in understanding and extending gene targeting technology to species other than the mouse. Although the dominant DSB repair pathway in the cell, NHEJ, appears to direct the mode of exogenous DNA integration in a host genome, the molecular mechanisms of foreign DNA integration in mammalian cells is still not resolved and requires further investigation.

As shown in Example 1, that the transient, RNA interference (RNAi)-induced deficiency of Ku70 and Xrcc4 proteins in human cells reduces exogenous DNA integration. By transient down regulation of key NHEJ components in cell culture using RNAi, loss-of-function phenotypes can be assessed irrespective of an essential role for any given protein. The objectives of this study were to assess the transient Ku70 and Xrcc4 loss-of-function and the subsequent consequences on the integration and gene targeting of exogenous DNA in the host genome. Here we demonstrated that random DNA integration can be reduced by the depletion of NHEJ proteins and HR increased, thereby highlighting potential applications in gene targeting and gene and cancer therapy.

Materials and Methods

Small interfering RNA (siRNA). Two distinct stealth, small interfering RNA (siRNA) duplex oligoribonucleotides targeted against human Ku70 (Gene Bank no. NM001469) and human Xrcc4 (Gene Bank no. NM003401) were synthesized by Invitrogen. The sequences for Ku70 siRNA were:

| | |
|---|---|
| (i) | sense 5′-CCUCCAAUAAAGCUCUAUCGGGAAA-3′ |
| (ii) | sense 5′-GGAGUCGUCAGAUUAUACUGGAGAA-3′. |

The sequences for Xrcc4 were:

| | |
|---|---|
| (iii) | sense 5′-CCACCUUGUUUCUGAACCCAGUAUA-3′ |
| (iv) | sense 5′-GGAAGCUUUGGAGACUGAUCUUUAU-3′. |

The siRNA sense and antisensense strands were mixed in equimolar ratios, annealed and transfected into human HCT116 colon cancer cells using Lipofectamine 2000 (Invitrogen) according to the manufacturer's protocol. A Stealth siRNA negative control (Invitrogen) (give sequence) not homologous to any known gene was used to ensure against induction of nonspecific cellular events caused by the transfection of the siRNA into cells. Also, a BLOCK-iT (Invitrogen) fluorescent siRNA was used to verify transfection efficiency. The two siRNAs for each protein, Ku70 and Xrcc4, were tested and the ones that were most efficient in achieving the desired protein knock-down with the smallest oligo concentration were selected for experimental use.
Small interfering RNA (siRNA). In addition to using single Stealth™ siRNA a mixture of 21mer siRNA prepared from segments of the human Ku70 (167 bp in exon 5 from sequence position 407 to 574; and 249 bp in exon 13 from nucleotides 1764 to 1989; Gene Bank accession no. NM001469) and Xrcc4 (172 bp in exon 2 from sequence position 317 to 489; and 108 bp in exon 6 from nucleotides 858 to 1067; Gene Bank accession no. NM003401) genes were chosen to generate double stranded RNA using the BLOCK-iT T7-TOPO® Linker (Invitrogen) for subsequent “Dicing” to create pools of siRNA using BLOCK-iT™ Dicer RNAi kit (Invitrogen) and stored at −80° C. The siRNA generated from both fragments for each gene were pooled.
Cell culture. Human colon cancer cells HCT116 (ATCC, Manassas, Va., USA) were cultured as monolayers in McCoy's 5A medium supplemented with 10% fetal bovine serum (Sigma Chemical) and antibiotic-antimycotic solution (penicillin-streptomycin-amphotericin b, Invitrogen). Cells were maintained in a humidified atmosphere at 37° C. in 5% CO₂. Prior to siRNA transfection cells were grown in antibiotic-antimycotic-free media.
SiRNA and DNA transfections.
HCT116 cells were plated at 30% confluence in 100 cm² culture plates. The following day, siRNA fragments (200 nM for Ku70, 100 nM for Xrcc4 and 100 nM non-specific siRNA as negative control) and Lipofectamine 2000 (1%, v/v, Invitrogen) were prepared in OptiMEM I (Invitrogen), according to the manufacturer's instructions, and added to cells in a total of 3 ml solution. After 4-6 h, 5 volumes of culture medium were added to the siRNA-containing medium.

To quantify HPRT gene targeting, four independent control pools (four of each: untransfected cells; mock-transfected and non-specific siRNA) and four independent siRNA treated cells (four from Ku70 and four from Xrcc4 transfections) were used in each experiment. A SalI-digested pHPRThyg targeting construct (20 μg/plate) was transfected into HCT116 cells 48 hours after the siRNA transfection by the same delivery method described for siRNA. Cells from each treatment group were plated out at 2.0×10⁶ cells in a 100 cm² plate and treated with hygromycin (100 μg/ml) from day 2 after transfection, and with hygromycin (100 μg/ml) plus 6-thioguanine (6-TG) (15 μg/ml) starting at day 3 after transfection. Random integration of the targeting construct was quantified in triplicate dishes containing 105 transfected cells each, selected with hygromycin. Targeted insertions were quantified by counting 6-TG resistant colonies 14 days after the start of selection. Colonies were stained with crystal violet at the end of the selection procedure. Resistant colonies were screened for correct gene targeting by PCR.

Immunoblots. The knockdown of specific targeted gene products was monitored by quantitative Western blots as described in Bertolini et al. (in press), using protein specific, commercially available antibodies. Briefly, the protein blots were developed with the ECL-Plus detection system (Amersham Pharmacia Biosciences), after which the labeled protein was visualized on a X-ray film. Band intensities were measured densitometrically by using the Alphaease FC software (Alphainnotech Corporation). Protein was quantitated relative to GAPDH levels on the same blots.

DNA analysis of transfected colonies. To evaluate the occurrence of HR and the structure of the HPRT locus following transfection, genomic DNA was extracted from independent hygromycin-resistant and hygromycin/6-TG-resistant clones and subjected to PCR analysis with the primers 5′-tttttt-3′ and 5′ttttt-3′. The oligonucleotides primers were designed to amplify a 2.5-kb fragment diagnostic of gene targeting at the exon II (FIG. 1). Correct gene targeting yielded positive colonies with only a 2.5 kb band. If the construct integrated by illegitimate recombination as shown in the hygromycin selected colonies the PCR reaction produces a 2.5 kb band from the construct plus a 200 bp fragment from the intact HPRT endogenous allele. Clones were scored as targeted at the HPRT locus if only the 2.5 kb band was detected, and counted as random events if both HPRT-derived, 2.5-kb and 200 bp bands were observed.

Results

Knockdown of Ku70 and Xrcc4 by siRNA.

Two distinct siRNA were designed and tested, corresponding to different regions of Ku70 and Xrcc4 mRNA. The siRNA were transfected by lipofection and assayed for protein levels by quantitative Western blotting 48 h after transfection, using anti-human Ku70 and Xrcc4 antibodies with GAPDH as an internal control. Treatment of HCT116 cells with both siRNA fragments targeting Ku70 and Xrcc4 transcripts resulted in significant down regulation of the respective proteins to approximately 15% of normal levels at 48 h post-transfection (P<0.05). The HCT116 cells were transfected with 200 nM of siRNA corresponding to the Ku70 mRNA sequence at nucleotide position 920 and with 100 nM for Xrcc4 mRNA corresponding to nucleotide position 152 in all subsequent experiments.

Treatment of cells with no siRNA (sham transfection, not shown) and with a non-specific, negative control siRNA did not affect the relative abundance of Ku70, Ku86 or Xrcc4 proteins at 48 post-transfection, with all the levels of these proteins being comparable with the levels observed in non-transfected cells.

Effect of Ku70 and Xrcc4 Depletion on DNA Integration and Gene Targeting

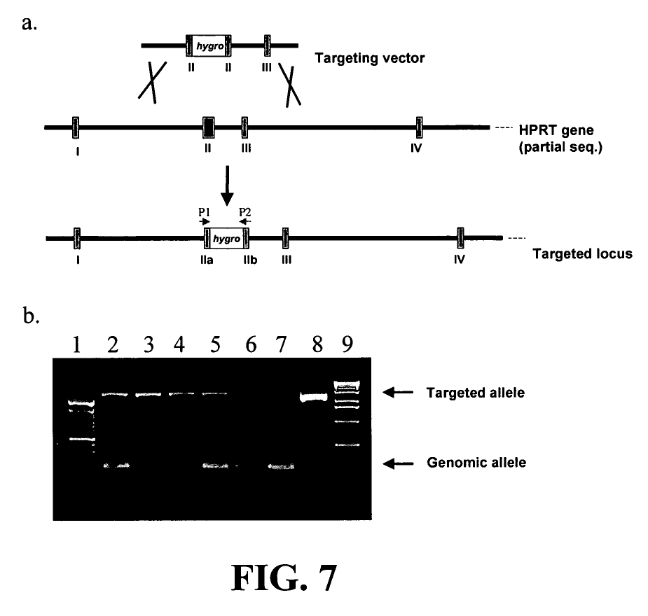

An HPRT-based system was used to quantify gene targeting in human cells (FIG. 7). HPRT is an X-linked, single-copy gene in diploid human male cells, and its inactivation leads to 6-thioguanine (6-TG) resistance. After transfection of pHPRThyg into hct116 cells random, or illegitimate insertion, and gene targeting were quantified as the number of colonies resistant to hygromycin only or resistant to both hygromycin and 6-TG, respectively. Using this system, gene targeting frequencies in untreated cells were approximately 1×10⁻⁶ targeted clones per transfected cell, while the ratio of gene targeting:nonhomologous recombination was approximately 1:200.

To test whether the depletion of NHEJ proteins promotes gene targeting we quantified this process in the pools of HCT116 cells transiently depleted of Ku70 or Xrcc4 protein. In each of two separate experiments, four independent pools of control cells or siRNA treated cells were transfected with pHPRThyg to estimate the frequencies of gene targeting at the HPRT locus and of random integration. We observed a 3.3 to 3.9-fold increase in the absolute frequency of gene targeting (ratio of homologous recombinants to total number of transfected cells) of HPRT upon Ku70 and Xrcc4 protein depletion (Table 4).

TABLE 4
pHPRT transfection efficiency in HCT116 cells (siRNA dicer)

| Treatment | Random | Homologous | RI/GT |
|---|---|---|---|
| Control | 2.01 × 10⁻⁴ | 0.9 × 10⁻⁶ | 223.3 |
| Sham | 1.60 × 10⁻⁴ | 1.2 × 10⁻⁶ | 133.3 |
| Ku70 | 0.68 × 10⁻⁴ | 3.5 × 10⁻⁶ | 19.4 |
| siRNA | (↓ 66%) | (↑ 3.9) | (↑ 12) |
| Xrcc4 | 0.57 × 10⁻⁴ | 3.0 × 10⁻⁶ | 19.2 |
| siRNA | (↓ 72%) | (↑ 3.3) | (↑ 12) |

TABLE 5
Steath data

| Treatment | Random | Homologous | RI/GT |
|---|---|---|---|
| Control | 5.15 × 10⁻⁴ | 7.6 × 10⁻⁷ | 730 |
| Sham | 5.03 × 10⁻⁴ | 7.05 × 10⁻⁷ | 709 |
| Ku70 | 1.63 × 10⁻⁴ | 8.76 × 10⁻⁷ | 187 |
| siRNA | (3.5 × ↓) | (1.5↑) | (↑4) |
| Xrcc4 | 2.8 × 10⁻⁴ | 1.26 × 10⁻⁶ | 212 |
| siRNA | (2 × ↓) | (2↑) | (↑4) |

In addition, random integration frequencies were significantly decreased by 66 to 72% relative to controls. When compared to the sham transfected cells these ratios changed to a 2.5 to 2.9 fold increase in HR and a 58 to 64% decrease in non-homologous insertions. This is reflected in an average 12-fold increased in the ratio gene targeting: nonhomologous recombination, from approximately 1:200 in control cells to approximately 1:20 in Ku70 or Xrcc4 depleted cells and gene targeting frequency approximately 3×10⁻⁶ targeted clones per transfected cell. The decrease of illegitimate integration and the increase in gene targeting are statistically significant within experiments (t test, Table 4). We obtained P values of <0.0001 in the ANOVA for the increases in gene targeting and the ratio of gene targeting:nonhomologous recombination in NHEJ depleted cells.

Discussion

Homologous recombination of a transfected DNA with its endogenous genomic sequence is a very inefficient processes in mammalian somatic cells compared to random integration, yet the importance of genetic engineering and gene targeting is undeniable in our era. This work is directed towards two goals; first to develop methods to increase the efficiency of gene targeting in human and animal cells and secondly, to contribute to a better understanding of foreign DNA integration into the genome.

To evaluate whether the down-regulation of key factors in the NHEJ pathway could increase the frequency of gene targeting in human cells we set up a system where HCT116 cells were transiently depleted of Ku70 and Xrcc4 proteins. We have previously demonstrated that the transient depletion of Ku70 or Xrcc4 is sufficient to develop a phenotype, both with respect to increased radiosensitivity as well as decreased expression of a transfected, linear GFP construct. (Bertolini et al, in press). In the latter case the decreased expression of the transfected GFP construct was interpreted as evidence that the NHEJ pathway was sufficiently impaired to decrease the non-homologous insertion of the construct and/or re-circularization of the plasmid. In this work, we used a HPRT targeting vector to not only quantify the decrease in random insertion but also to test the hypothesis that gene targeting would be increased in human cells when the NHEJ pathway was impaired.

In mammalian somatic cells DSBs in DNA are predominantly repaired by the NHEJ pathway. Inhibition of this pathway may result in increased HR, taking into consideration the leading model of recombination that homologous and non-homologous pathways compete for a common DNA substrate (VanDyck et al., 1999; Allen et al., 2003). The reduction in the availability of NHEJ pathway enzymes when KU70 or Xrcc4 are depleted would make DNA substrate available for HR enzymes to act. Our description of a reduced level of random DNA integration into the genome of cells deficient in NHEJ proteins (Bertolini et al, in press), is in agreement with the hypothesis that exogenous DNA integration also follow the preferred mechanism of DNA repair in the cell.

HCT116 cells transiently depleted of Ku70 and Xrcc4, two key players in the NHEJ pathway, show up to a 65% decrease in random DNA integration events as determined by the hygromycin-selected colonies. A potential explanation for reduced integration in NHEJ-depleted cells is that foreign DNA is degraded more rapidly (Liang et al., 1996), consistent with NHEJ components protecting exogenous DNA from cellular nucleases.

The HCT116 cells with transient depletion of the Ku70 and Xrcc4 proteins also showed a 3 fold stimulation in the absolute gene targeting frequency compared with controls or sham transfected cells. The increase in HR is presumably due to the lack of NHEJ proteins. HCT116 are fast growing cells with a large proportion of cells in S/G2 phase of the cell cycle, when HR is more likely to occur due to enzyme availability.

From this study, we conclude that the depletion of Ku70 and Xrcc4 proteins in HCT116 cells decreased significantly illegitimate recombination and increased the frequency of gene targeting at the HPRT locus. Overall the decrease in random integration, coupled with the increase in HR, resulted in a 12-fold increased gene targeting efficiency.

### REFERENCES

1. Lin, Y. and Waldman, A. S. (2001) Genetics, 158:1665-74.
2. Dellaire, G., Ju Yan, J., Little, K. C., Drouin, R. and Chartrand, P. (2002) Chromosoma, 111:304-12.
3. Jasin, M. (1996) Trends Genet., 12:224-8.
4. Smith, K. (2001) Theoretical mechanisms in targeted and random integration of transgene DNA. Reprod. Nutr. Dev., 51: 465:485.
5. Van Gent, D. C., Nat. Rev. Genet., 2, 196-206.
6. Jackson, S. P. (2002) Carcinogenesis, 23, 687-696.
7. Ristic, D., Modesti, M., Roland Kanaar, R. and Wyman, C. (2003) Rad52 and Ku bind to different DNA structures produced early in double-strand break repair. Nucleic Acids Research, 31:5229-5237
8. Feng Liang, F., Peter, J. Romanienko, P. J., David T. Weaver, D. T., Jeggo, P. A. and Jasin, M. (1996) Proc Natl Acad Sci U S A, 93:8929-33.
9. Kunzelmann, K., Legendre, J. Y., Knoell, D. L., Escobar, L. C., Xu, Z. and Gruenert, D. C. (1996) Gene Ther., 3:859-67.
10. Kim, P. M., Allen, C., wagener, B. M., Shen, Z and Nickoloff, J. A., (2001) Nucleic Acids Res., 29:4352-4360.
11. Yanez, R. J. and Porter, A. C. (2002) Nucleic Acid Res., 30:740-748.
12. Johnson, B. L., Thyagarajan, B., Krueger, L., Hirsch, B. and Campbell, C. (1996) Mutat. Res., 363:179-189.
13. Hacein-Bey-Abina, S., Von Kalle, C., Schmidt, M., McCormack, M. P., Wulffraat, N., Leboulch, P., Lim, A., Osborne, C. S., Pawliuk, R., Morillon, E., et al. (2003) Science 302:415-419.
14. Jasin, M., de Villiers, J., Weber, F., Schaffner, W. (1985) Cell, 43:695-703.
15. Deng, C. and Capecchi, M. R., (1992) Mol. Cell. Biol., 12:3365-3371.
16. Scheerer, J. B. and Adair, G. M. (1994) Mol. Cell. Biol.; 14:6663-6673.
17. Smith, F., Rouet, P., Romanienko, P J. And Jasin, M. (1995) Nucleic Acid Res., 23:5012-5019.
18. Russel, D. W. and Hirata, R. K. (1998) Nat. Genet., 18:325-330.
19. McCormack, M. P. and Rabbitts, T. H. (2004) N. Engl. J. Med. 350:913-922.
20. Di Primio, C., Galli, A., Cervelli, T., Zoppè, M. and Rainaldi, G. (2005) Nucleic Acids Res., 33:4639-4648.
21. Krappmann, S., Sasse, C. and Braus, G. H. (2006) Eukaryot Cell., 5:212-5.
22. Takahashi, T., Masuda, T. and Koyama, Y. (2006) Mol. Genet. Genomics., 275:460-70.
23. Ninomiva, Y., Suzuki, K., Ishii, C. and Inoue, H. (2004) Proc Natl Acad Sci U S A, 17:12248-12253
24. Hefferin, M. L. and Tomkinson, A. E. (2005) DNA Repair, 4:639-648.
25. Secretan, M. B., Scuric, Z., Oshima, J., Bishop, A. J., Howlett, N. G., Yau, D. and Schiestl, R. H. (2004) Mutat Res., 554:351-364.
26. Goedecke, W., Eijpe, M., Offenberg, H. H., Van Aalderen, M., Heyting, C. (1999) Nat. Genet., 23:194-198.
27. Van Dyck, E., Stasiak, A. Z., Stasiak, A. and West, S. C. (1999) Nature, 398:728-731.
28. Hamilton, A. A. and Thacker, J. (1987) Mol Cell Biol., 7:1409-1414.
29. Moore, P. D., Song, K. Y., Chekuri, L., Wallace, L. and Kucherlapati, R. S. (1986) Mutat Res., 160:149-155.
30. Dominguez-Bendala, J. Masutani, M. and Mcwhir, J. (2006) Cell Biol. International, 30:389-393
31. Li, L., Olvera, J. M., Yoder, K. E., Mitchell, R. S., Butler, S. L., Lieber, M., Martin, S. L. and Bushman, F. D. (2001) EMBO J., 20, 3272-3281.
32. Daniel, R., Greger, J. G., Katz, R. A., Taganov, K. D., Wu, X., Kappes, J. C. and Skalka, A. M. (2004) J. Virol., 78, 8573-8581.
33. Manivasakam, P., Aubrecht, J., Sidhom, S. and Schiestl, R. H. (2001) Nucleic Acids Res., 29, 4826-4833.
34. Lieber, M. R., Ma, Y., Pannicke, U. and Schwarz, K. (2003) Nat. Rev. Mol. Cell. Biol., 4, 712-720.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

That which is claimed:

1. A method for increasing the efficiency of homologous recombination of a polynucleotide of interest in a eukaryotic cell comprising
a) providing the eukaryotic cell having a decreased level of non-homologous recombination activity, wherein the decreased level of non-homologous recombination activity is transient;
b) introducing into said eukaryotic cell a composition comprising a homologous recombination cassette comprising a polynucleotide of interest and at least a first region having sufficient sequence identity to a corresponding first region of a target site in said eukaryotic cell, wherein said polynucleotide of interest is inserted into the target site by a homologous recombination event.

2. The method of claim 1, wherein said homologous recombination cassette comprises the polynucleotide of interest flanked by the first region and a second region having sufficient identity to a corresponding second region of the target site.

3. The method of claim 2, wherein said eukaryotic cell of step (a) comprises at least one polynucleotide comprising a silencing element, wherein said silencing element reduces the level of a gene product that contributes to non-homologous recombination.

4. The method of claim 3, wherein the gene product that contributes to non-homologous recombination encodes a Ku70 polypeptide or a Xrcc4 polypeptide.

5. The method of claim 3, wherein said silencing element comprises an siRNA silencing element, an miRNA silencing element, a double stranded RNA silencing element, a hairpin RNA silencing element, an antisense silencing element or a sense silencing element.

6. The method of claim 1, wherein said eukaryotic cell of step (a) further comprises at least one heterologous polynucleotide which when expressed in said eukaryotic cell increases homologous recombination activity in said eukaryotic cell.

7. The method of claim 1, wherein said composition of step (b) further comprises a polypeptide which increases homologous recombination activity in said eukaryotic cell.

8. A method for increasing the efficiency of homologous recombination of a polynucleotide of interest in a eukaryotic cell comprising
a) introducing into said eukaryotic cell a first polypeptide or a first polynucleotide which transiently decreases the level of non-homologous recombination in said eukaryotic cell; and,
b) introducing into said eukaryotic cell a composition comprising a homologous recombination cassette comprising a polynucleotide of interest and a first region having sufficient sequence identity to a corresponding first region of a target site in said eukaryotic cell, wherein said polynucleotide of interest is integrated into the target site by a homologous recombination event.

9. The method of claim 8, wherein said homologous recombination cassette comprises the polynucleotide of interest flanked by the first region and a second region having sufficient identity to a corresponding second region of the target site.

10. The method of claim 9, wherein said first polynucleotide of step (a) comprises a silencing element, said silencing element reduces the level of a gene product that contributes to non-homologous recombination.

11. The method of claim 10, wherein the gene product that contributes to non-homologous recombination encodes a Ku70 polypeptide or a Xrcc4 polypeptide.

12. The method of claim 9, wherein said polynucleotide encoding said silencing element is stably incorporated into the eukaryotic cell and is operably linked to an inducible promoter.

13. The method of claim 10, wherein said silencing element comprises an siRNA silencing element, an miRNA silencing element, a double stranded RNA silencing element, a hairpin RNA silencing element, an antisense silencing element or a sense silencing element.

14. The method of claim 8, wherein said target site is endogenous to said eukaryotic cell or said target site is heterologous to said eukaryotic cell.

15. The method of claim 8, wherein said target site is chromosomally located or extrachromosomally located in said eukaryotic cell.

16. The method of claim 8, wherein said eukaryotic cell is a mammalian cell.

17. The method of claim 8, wherein said method further comprises selecting said eukaryotic cell having said polynucleotide of interest integrated into the target site.

18. The method of claim 8, wherein said method further comprises introducing into said eukaryotic cell at least a second polynucleotide which when expressed in said eukaryotic cell increases homologous recombination activity in said eukaryotic cell.

19. The method of claim 18, wherein said second polynucleotide encodes a RecA polypeptide, a Rad54 polypeptide, or a Rad51 polypeptide.

20. The method of claim 8, wherein said composition of step (b) further comprises a polypeptide which increases homologous recombination activity in said eukaryotic cell.

21. The method of claim 20, wherein said polypeptide which increases homologous recombination activity in said eukaryotic cell is selected from the group consisting of a RecA polypeptide, a Rad54 polypeptide, or a Rad51 polypeptide.

22. A kit comprising a polynucleotide encoding a silencing element, wherein said silencing element when introduced into a eukaryotic cell reduces the level of a gene product that contributes to non-homologous recombination, and, increases the homologous recombination activity in said eukaryotic cell; said kit further comprises one or more polynucleotides selected from the group consisting of:
a) a polynucleotide comprising a homologous recombination cassette comprising at least a first region having sufficient sequence identity to a corresponding first region of a target site in said eukaryotic cell; or
b) a polynucleotide which when expressed in said eukaryotic cell increases homologous recombination activity in said eukaryotic cell.

23. The kit of claim 22, wherein said homologous recombination cassette comprises the first region and a second region having sufficient identity to a corresponding second region of the target site.

24. The kit of claim 22, wherein the gene product that contributes to non-homologous recombination encodes a Ku70 polypeptide or a Xrcc4 polypeptide.

25. The kit of claim 22, wherein said silencing element encoded by said polynucleotide comprises an siRNA silencing element, an miRNA, a double stranded RNA silencing element, a hairpin RNA silencing element, an antisense silencing element or a sense silencing element.

26. The kit of claim 22, wherein said polynucleotide which when expressed in said eukaryotic cell increases homologous recombination activity is selected from the group consisting of a polynucleotide encoding a RecA polypeptide, a Rad54 polypeptide, or a Rad51 polypeptide.

27. A kit comprising a eukaryotic cell having a decreased level of non-homologous recombination activity, wherein the decreased level of non-homologous recombination activity is transient, said kit further comprises one or more polynucleotides selected from the group consisting of:
a) a polynucleotide comprising a homologous recombination cassette; or
b) a polynucleotide which when expressed in said eukaryotic cell increases homologous recombination activity in said eukaryotic cell.

28. The kit of claim 27, wherein said eukaryotic cell comprises at least one heterologous polynucleotide stably incorporated into the cell comprising a silencing element operably linked to an inducible promoter, said silencing element reduces the level of a gene product that contributes to non-homologous recombination.

29. The kit of claim 28, wherein the gene product that contributes to non-homologous recombination encodes a Ku70 polypeptide or a Xrcc4 polypeptide.

30. The kit of claim 28, wherein said silencing element encoded by said polynucleotide comprises a siRNA silencing element, a miRNA, a double stranded RNA silencing element, a hairpin RNA silencing element, an antisense silencing element or a sense silencing element.

31. The kit of claim 27, wherein said polynucleotide which when expressed in said eukaryotic cell increases homologous recombination activity is selected from the group consisting of a polynucleotide encoding a RecA polypeptide, a Rad54 polypeptide, or a Rad51 polypeptide.

32. A method for increasing the efficiency of homologous recombination with an exogenous nucleic acid in a eukaryotic cell, the method comprising genetically modifying the cell with an expression construct that comprises a nucleotide sequence encoding an siRNA that specifically reduces the level of a gene product that contributes to non-homologous recombination, wherein the efficiency of homologous recombination is increased.
